(19) United States
(12) Patent Application Publication
(10) Pub. No.: US 2025/0051770 A1
(43) Pub. Date: Feb. 13, 2025

(54) COMPOSITIONS COMPRISING THERAPEUTIC NUCLEIC ACID AND SAPONIN FOR THE TREATMENT OF MUSCLE-WASTING DISORDERS

(71) Applicant: SAPREME TECHNOLOGIES B.V., Bilthoven (NL)

(72) Inventors: Miriam Verena BUJNY, Bilthoven (NL); Ruben POSTEL, Bilthoven (NL); Guy HERMANS, Bilthoven (NL)

(21) Appl. No.: 18/723,163

(22) PCT Filed: Dec. 20, 2022
(86) PCT No.: PCT/NL2022/050735

§ 371 Date: Jun. 21, 2024

(30) Foreign Application Priority Data
Dec. 22, 2021 (NL) 2030217

(51) Int. Cl. C12N 15/113; A61K 31/704; A61K 36/185; A61K 47/54; A61K 47/61; A61K 47/64; A61K 47/68; A61P 21/00

(52) U.S. Cl. CPC C12N 15/113; A61K 31/704; A61K 36/185; A61K 47/549; A61K 47/61; A61K 47/64; A61K 47/642; A61K 47/644; A61K 47/6849; A61K 47/6889; A61P 21/00; C12N 2310/11; C12N 2310/314; C12N 2310/315; C12N 2310/321; C12N 2310/3233

(57) ABSTRACT

The invention lies in the field of treatment and prophylaxis of muscle wasting disorders, in particular the ones involving a genetic factor that can be targeted by a delivery of a therapeutic nucleic acid into the muscle cells. In line with the latter aspect, disclosed herein are pharmaceutical compositions and advantageous components thereof that substantially enhance the effective delivery and release of a therapeutic nucleic acid into the right internal compartment of the muscle cell, such as the cytosol and/or the nucleus, in which compartment it can reach and act upon its genetic target. As disclosed herein, this substantially enhanced delivery and release is achieved by a provision of an endosomal-escape-enhancing saponin in the disclosed herein pharmaceutical compositions comprising therapeutic nucleic acids and, optionally, muscle cell-targeting ligands conjugated therewith. As for the first time demonstrated herein, these saponin types not only surprisingly retain their endosomal-escape-enhancing properties in fully differentiated muscle cells but also can in an unconjugated state successfully be delivered thereto together with the therapeutic nucleic acids.

### TECHNOLOGICAL FIELD

The invention lies in the field of treatment and prophylaxis of muscle wasting disorders, in particular the ones involving a genetic factor that can be targeted by a delivery of a therapeutic nucleic acid into the muscle cells. In line with the latter aspect, disclosed herein are pharmaceutical compositions and advantageous components thereof that substantially enhance the effective delivery and release of a therapeutic nucleic acid into the correct internal compartment of the muscle cell, such as the cytosol and/or the nucleus, in which compartment it can reach and act upon its genetic target. As disclosed herein, this substantially enhanced delivery and release is achieved by a provision of an endosomal-escape-enhancing saponin in the disclosed herein pharmaceutical compositions comprising therapeutic nucleic acids and, optionally, muscle cell-targeting ligands conjugated therewith. As for the first time demonstrated herein, these saponin types not only surprisingly retain their endosomal-escape-enhancing properties in fully differentiated muscle cells but also can in an unconjugated state successfully be delivered thereto together with the therapeutic nucleic acids.

### BACKGROUND

Muscle wasting disorders represent a major cause of human diseases worldwide. They can be caused by an underlying genetic condition, such as in various types of muscular dystrophies or congenital myopathies [Cardamone, 2008], or can be related to aging, like the age-related loss of muscle mass known as sarcopenia, or result from a traumatic muscle injury, among others.

Both the hereditary and non-hereditary muscle wasting disorders primarily manifest as a debilitating weakening or loss of striated muscle tissue, which is the tissue responsible, among others, for whole-body oxygen supply, metabolic balance, and locomotion. The striated muscle tissue is built by two types of striated muscle cells, namely the skeletal muscle cells and the cardiac muscle cells [Shadrin, 2016]. Skeletal muscles comprise 30 to 40% of total human body mass and can regenerate in response to small muscle tears that occur during exercise or daily activity owing to the presence of resident muscle stem cells called satellite cells (SCs), which upon injury activate, proliferate, and fuse to repair damaged or form new muscle fibres [Dumont, 2016]. In contrast, cardiac muscle does not possess a cardiomyogenic stem cell pool and has little to no regenerative ability, with injury resulting in the formation of a fibrotic scar and, eventually, impaired pump function [Uygur, 2016].

Both of these cell types are terminally differentiated and highly structurally- and functionally-specialised and these characteristics usually correlate with an increased difficulty of targeting payloads into such cells' inner compartments. In particular, muscle cells are covered by a unique type of a cell membrane termed sarcolemma that, just like in neurons, is excitable. Furthermore, these cells are particularly resilient characterised by contractibility, extensibility, and elasticity, which are the key features required for fulfilling their primary function in the muscle tissue, which is the production of tension resulting in the generation of force that contracts the muscle cells in order to produce voluntary or involuntary movement of different body parts.

In line with this, despite a widespread recognition that certain therapeutic payloads can yield beneficial effects when delivered into the muscle cells, it is also very well known that targeting these cells has proven notoriously challenging, as acknowledged in e.g. WO2021142227. This is particularly true for cardiac muscle cells where any on- and off-target activity of a drug cause serious safety liabilities [Slordalm 2006]. Systemic delivery to heart of even specifically muscle-targeted therapeutics is known to have very limited efficacy, and even direct intramuscular injections into the heart have shown restricted heart exposure [Ebner, 2015].

In addition to the above limitations, very few treatment options and strategies are available for individuals suffering from acquired advanced-stage or genetic muscle wasting disorders. In fact, for majority of them, there are no drugs available with palliative treatment frequently being the only applicable solution to ease the suffering of the affected individual.

In particular, an incredible large spectrum of muscle cell-related genetic disorders (sometimes collectively termed as hereditary myopathies) has been described to date, and although due to relatively low prevalence majority of them are catalogued as “rare diseases”, the sum of the different forms makes these disorders a relatively common health problem that affects the life quality of millions of patients worldwide, causing debilitating complications that frequently lead to death [González-Jamett, 2017].

One common classification of muscle cell-related genetic disorders is based on the location of the mutated protein product originating from the muscle cell. Namely, congenital myopathies are considered to be caused by genetic defects in the contractile apparatus within the muscle cell, and are defined by distinctive static histochemical or ultrastructural changes on muscle biopsy. In contrast, muscular dystrophies are described as diseases of the muscle membrane or its supporting proteins and are generally characterised by pathological evidence of ongoing muscle degeneration and regeneration. [Cardamone, 2008].

In brief, the contractile apparatus includes myofibrils comprised of actin and myosin that form myofilaments which slide past each other producing tension that changes the shape of the muscle cell. The function of the contractile apparatus heavily relies on its interaction with the reinforced muscle cell cytoskeleton and the highly specialised structures within and around the sarcolemma that, unlike most of the cell membranes in the human body, is heavily coated by a polysaccharide material termed glycocalyx that contacts the basement membrane around the muscle cells. This basement membrane contains numerous collagen fibrils and specialized extracellular matrix proteins such as laminin. The matrix proteins provide a scaffold to which the muscle fibre can adhere. Through transmembrane proteins in the sarcolemma, the actin skeleton inside the muscle cells is connected to the basement membrane and the cell's exterior. Such anchored numerous muscle cells make up the muscle tissue and by synchronous and controlled production of tension they can generate significant force.

This structural and functional complexity of the muscle cells, including their intracellular contractile apparatus, the network of proteins reinforcing and accounting for specific function and architecture of the sarcolemma, and the multi-component scaffolding outside of it, is a product of a large muscle cell-specific proteome.

A substantial part of this proteome are large structural proteins that are translated from purely-muscle-cell-specific transcripts originating from frequently very large multi-exonic genes that tend to undergo extensive alternative splicing events [Savarese, 2020]. In fact, various mutations scattered along some of the largest genes of the human genome, notably including DMD, TTN, NEB, RYR1, are recognized as underlying causes of the best characterised muscle cell-related genetic disorders.

Perhaps and arguably the most investigated genetic muscle cell-related disorder is the Duchenne muscular dystrophy (DMD) that results from a mutation in the DMD gene encoding for dystrophin protein, which prevents the production of the muscle isoform of dystrophin (Dp427m). DMD is a particularly severe disease characterized by progressive wasting and replacement of skeletal muscles with fibrous, bony, or fatty tissue, which eventually leads to death due to usually heart-muscle or respiratory failure. DMD is recessive and X-chromosome-linked (X-linked). Consequently, most patients are males. On average, they develop the earliest symptoms around 2-3 years of age, become wheelchair dependent around 10-12 years, and with even with optimal care die between 20 and 40 years of age. The different spectra can be explained by the fact that DMD is not caused by a precise defined site-specific or single hot-spot mutation in the DMD gene. To the contrary, DMD, like many other muscle-cell related genetic disorders caused by different mutations in large multi-exonic genes, can be seen as a spectrum of disorders which severity of the phenotype depending on the extent to which the reading frame of transcript was affected. DMD cases usually harbour frameshifting or nonsense mutations that cause premature truncation leading to non-functional and unstable dystrophin. In contrast to this, a milder dystrophinopathy called Becker muscular dystrophy (BMD) is caused by in frame mutations of the DMD gene, i.e. mutations that maintain the reading frame and lead to a production of a dystrophin mutant protein that is merely internally truncated.

Based on the observation that most out-of-frame mutations result in severe DMD, whereas the vast majority of in-frame mutations result in milder BMD, different antisense oligonucleotide (ASO)-based therapeutics have been tested and developed for DMD with the aim of restoring the reading frame of dystrophin transcripts causing the production of a protein that is at least partially functional. These ASOs are short (20-30 nucleotides), frequently chemically-modified nucleic acids or nucleic acid analogues that specifically bind to a target exon during pre-mRNA splicing causing a so called skipping of the faulty exon, i.e. prevention of its inclusion into the mRNA. The exon-skipping-ASO approach is mutation specific as different exons need to be skipped depending on the mutation location. However, skipping of certain exons is applicable to larger groups of patients, including the skipping of exon 51 (14%), exon 45 (8%), exon 53 (8%), and exon 44 (6%) [Bladen, 2013]. To date four different morpholino-type ASOs that are designed to skip exon 51 (eteplirsen), exon 53 (golodirsen and viltolarsen), or exon 45 (casimersen) have shown some evidence of induced dystrophin restoration in small cohorts of patients and, despite exhibition of certain systemic side-effects, were granted FDA-approvals. Another exon 51-skipping ASO with a 2′-O-methyl phosphorothioate modification (drisapersen) was evaluated in placebo-controlled trials but eventually was not approved by the FDA, due to e.g. occurrence of injection site reactions, proteinuria and, in a subset of patients, thrombocytopenia [Goemans, 2018]. Alternative compositions for induction of exon skipping can be found in WO2018129384.

Other nucleic acid-based approaches in DMD included attempted delivery of micro-dystrophin cDNA at high vector dose, for which clinical trials are under way with some already reported success of micro-dystrophin expression but not without observation of severe adverse effects in a subset of patients, including transient renal failure likely due to an innate immune response [Mendell, 2020]. Alternatively, efforts are also ongoing to deliver cDNA of genes that encode proteins that can improve muscle mass, such as follistatin [Mendell, 2020] or that target disease mechanisms, such as SERCA2a [Wasala, 2020] f. Further considerations involve the use of microRNAs, miRNA mimic products, antimiRs, antagomiRs and the line either alone or for co-administration with other nucleic acid-based therapies for the stimulation of growth and/or regeneration of the wakened muscle cells [Arenega, 2021]. For example, WO2018080658 discloses miR-128-1 as LNA-based ASO therapeutic for the treatment of DMD. Yet a further alternative approach was proposed based on CRISPR/Cas9 technology with guide RNAs designed for restoring the reading frame e.g. by exon deletion or by abolishing of a splice site, a proof of concept of which was tried in DMD cell lines and animal models [Chemello, 2020; Nelson 2017]. However, all the genome-editing work is still in a preclinical phase and multiple challenges have to be overcome to apply it systemically in humans, including optimal delivery of the genome-editing components.

Thousands of different mutations in DMD have been found in patients with DMD or BMD [Blanden, 2015]. Similar situation exists for many other genetic muscle cell-related disorders where gene mutation targets are identified. These include but are not limited to other muscular dystrophies including facioscapulohumeral muscular dystrophy (affected genes: DUX4/double homeobox 4), myotonic dystrophy (DMPK), Emery-Dreifuss muscular dystrophy (affected genes: EMD/emerin and LMNA/lamin A/C), limb-girdle muscular dystrophy 1 (affected genes: MYOT/myotilin, LMNA/lamin A/C etc.), congenital muscular dystrophy (affected genes: LAMA2/merosin or laminin-α2 chain/any of COL6A genes encoding for collagen 6A); or dilated familial cardiomyopathy (affected genes: LMNA/lamin A/C), as well as congenital myopathies notably including nemalin myopathy (affected genes: NEB/nebulin, ACTA/skeletal muscle alpha-actin, TPM3/alpha-tropomyosin-3, TPM2/beta-tropomyosin-2, TNNT1/troponin T1, LMOD3/leiomodin-3, MYPN/myopalladin etc.) or congenital fibre-type disproportion myopathy (affected genes: TPM3/alpha-tropomyosin-3, CTA/skeletal muscle alpha-actin, RYR1/ryanodine receptor channel), as well as any syndromes involving mutations e.g. in TTN gene (titin). Consequently, due to the variability of the mutations in even defined genetic targets underlying many different muscle wasting disorders, use of therapeutic nucleic acids, such ASOs or antagomirs [Cerro-Herreros, 2020], appears to be a logical and practical way forward for the development of new therapies for various muscle-wasting disorder.

However, as already explained above, despite considerable advantages, even when coupled with various delivery systems, it is difficult to efficiently bring such nucleic acid-based therapeutics into the appropriate compartments inside the muscle-cells, like for example into the muscle cell cytosol for antisense-therapy or, therefrom, into the nucleus for direct gene-editing. This low efficiency of muscle cell transfection and in vivo naturally results in the concentrations of nucleic acid-based therapeutics being too low at their target site for achieving effective and sustained outcomes. This in turn results in the need to increase the administered dose, which then causes off-target effects. Most common of such side-effects include activation of the complement cascade, the inhibition of the clotting cascade, and toll-like receptor mediated stimulation of the immune system. Naturally, these effects are highly undesired and bear the risk of inducing health- or even life-threatening side effects in the patient including organ failure. The occurrence of such and similar adverse events had caused many nucleic acid-based therapeutics, like the DMD exon-skipping ASO drisapersen, to fail in clinical trials.

Therefore, there is a strong desire to provide novel nucleic acid-based drug formulations for the treatment of muscle cell wasting disorder, which in particular would show efficient nucleic acid-delivery rates into the muscle cells in vivo. Consequently, formulations are needed wherein the effect of the therapeutic nucleic acid would be (1) highly specific for its genetic target implicated in or causing a muscle cell wasting disorder, (2) sufficiently safe, (3) efficacious, (4) specifically directed to the muscle cells with little to none off-target activity on other cells, (5) have a sufficiently timely mode of action (e.g. the administered drug should reach the targeted site in the human patient within a certain time frame and should remain at the targeted site for a certain time frame), and/or (6) have sufficiently long lasting therapeutic activity in the patient's body, amongst others.

A comprehensive review of different drug delivery approaches into the striated muscle cells can be found in DC Ebner et al., 2015, Curr Pharm Des, 21(10):1327-36. doi: 10.2174/1381612820666140929095755, which mentions muscle targeting peptides, microbubbles, nanoparticles, viral-, transporter-, and antibody-based targeting techniques as well as highlights the fact that cellular uptake remains a major issue in muscle cells in general. With regard to the transporter-, and antibody-based targeting techniques, delivery of nucleic acids by a ligand-mediated targeting of endocytic (or internalizing) receptors on muscle cell surface is e.g. shown in WO2018129384 or WO2020028857.

However, to the inventors' knowledge and experience, none of the known muscle-specific delivery approaches achieves all or at least a substantial part of the above outlined beneficial characteristics (1)-(6). Consequently, despite the long-lasting and intensive research and the progress made in several areas of the field individually, there still exists a dire need for improvement of the efficiency of delivery of nucleic acid-based therapeutics into the muscle cells of the patients suffering from muscle wasting disorders.

### SUMMARY

To address this need, the inventors have developed and hereby describe novel pharmaceutical compositions comprising muscle cell-targeted therapeutic nucleic acids in combination with triterpenoid saponins of the 12,13-dehydrooleanane type. These specific saponin types were described in multi-component conjugates disclosed e.g. in WO2020126620, where they were described as possessing an endosomal-escape enhancing activity towards various antibody-drug conjugates (ADCs) in several cancer cell types.

Further in the context of tumour cells, these saponins were mentioned in e.g. WO2020126609 that describes the silencing of the HSP27 gene in various tumour models, with a combination of a saponin and a BNA for silencing HSP27 or with a combination of a saponin with a conjugate of a monoclonal antibody directed to a tumour-cell marker and a BNA for silencing HSP27.

Terminally differentiated muscle cells however, cardiac muscle cells in particular, are much different in metabolism as well as in cell membrane architecture and endocytic activity from the genetically unstable and constantly dividing tumour cells. Furthermore, due to perturbations in cell signalling pathways and chaotic proliferation activity, tumours are known to be supplied by permeable and leaky vascularisation [Hanahan and Weinberg, 2011], which is very different from the healthy and tight-junction-rich blood vessels that supply the muscle tissue.

Despite these differences, the inventors have observed a surprising and robust effect on exon-skipping efficiency in human and murine DMD transcript when combining muscle-endocytic-receptor-ligand-conjugated ASOs with the 12,13-dehydrooleanane type triterpenoid saponins. Without wishing to be bound by any theory, the inventors hypothesised that these findings indicate that said specific group of endosomal-escape enhancing saponins is very much capable of stimulating efficient exiting of the therapeutic nucleic acids' from the muscle cells' endosomes into the appropriate muscle cell inner compartments (in a very much desired for therapeutics but not fully understood phenomenon termed endosomal escape).

To the inventors' knowledge, the only instance of combining saponins with nucleic acids for the treatment of muscle cell wasting disorders was attempted by Wang et al. [Wang, 2018, Molecular Therapy: Nucleic Acids; Wang, 2018—Drug Design, Development and Therapy]. In these reports, the authors however concentrated on membrane-piercing transfection activity of non-covalently bound and non-targeted nucleic acid complexes with mainly steroidal saponins such as the known in vitro transfection agent digitonin. None of the saponins as investigated by Wang et al. however was an endosomal-escape enhancing saponin of the 12,13-dehydrooleanane type, and none of the complexes was specifically muscle-cell targeted via an endocytic receptor ligand.

In sum, to address the drawbacks of the prior art, presented herein are novel pharmaceutical compositions for the use in in the treatment or prophylaxis of a muscle wasting disorders, muscle cell-related genetic disorders in particular, the compositions comprising endosomal-escape-enhancing saponins of the 12,13-dehydrooleanane-type and a therapeutic nucleic acid advantageously conjugated with a muscle-specific endocytic receptor targeting ligand. The inventors observed that the described herein saponins have the unique ability to efficiently deliver the therapeutic nucleic acids into striated muscle cells, possibly by facilitating the endosomal escape of the nucleic acids specifically in the target muscle cells The findings presented herein open the venue of developing novel potentially low-therapeutic-load and thus safer treatment methods for the patients suffering from muscle wasting disorders. These and other advantages are presented further in the continuation.

In line with the above-described promising findings, disclosed herein are improved biologically active compounds and pharmaceutical compositions comprising nucleic acids and free-form (i.e. non conjugated with a macromolecule) endocytic-escape-enhancing 12,13-dehydrooleanane-type saponins with an aldehyde group at position C-23 of the saponin's aglycone core structure.

The disclosed herein conjugates possess the particular advantage of exhibiting the highly desired property of enhanced and effective delivery of therapeutic nucleic acids, such as antisense oligonucleotides, into differentiated muscles cells, striated muscle cells in particular, notably including heart muscle cells.

The innovative concepts presented herein will be described with respect to particular embodiments that should be regarded as descriptive and not limiting beyond of what is described in the claims. These embodiments as described herein can operate in combination and cooperation, unless specified otherwise.

It is one of several objectives of embodiments of present disclosure to provide a solution to the problem of insufficient delivery of nucleic acids into appropriate compartments of their target cells and of the related therewith non-specificity encountered when administering nucleic acid-based therapeutics to human patients suffering from a muscle-wasting disorder and in need of such therapeutics.

It is a further one of several objectives of the embodiments to provide a solution to the problem of insufficient safety characteristics of current nucleic-acid-based drugs, when administered to human patients in need thereof, in particular at side-effect inducing excessive doses.

It is yet a further one of several objectives of embodiments of the current invention to provide a solution to the problem of current nucleic acid-based therapies being less efficacious than desired, when administered to human patients in need thereof, due to not being sufficiently capable to reach and/or enter into to the diseased muscle cell with little to no off-target activity on non-diseased cells, when administered to human patients in need thereof.

At least one of the above objectives is achieved by providing a pharmaceutical composition for use in the treatment or prophylaxis of a muscle wasting disorder, in particular being a muscle cell-related genetic disorder such as congenital myopathy or a muscular dystrophy notably including Duchenne muscular dystrophy, the composition comprising
a nucleic acid, and
a saponin,
   wherein the saponin is a triterpenoid 12,13-dehydrooleanane-type saponin comprising an aldehyde group at position C-23 of the saponin's aglycone core structure under conditions present in endosomes and/or lysosomes of human cells.

In a further aspect, at least one of the above objectives is achieved by providing a therapeutic combination for a treatment or prophylaxis of a muscle cell-related genetic disorder, the therapeutic combination comprising:
(a) nucleic acid, and
(b) a saponin
wherein the saponin is a triterpenoid 12,13-dehydrooleanane-type saponin comprising an aldehyde group at position C-23 of the saponin's aglycone core structure under conditions present in endosomes and/or lysosomes of human cells.

In a further aspect, provided are further embodiments of the composition for the disclosed herein therapeutic or prophylactic uses and/or of the therapeutic combination according to the disclosure, which embodiments further address one or more of the above-stated objectives.

In particularly advantageous aspects, different embodiments of the disclosure are provided comprising advantageous components such as the ones selected from preferred sub-types of endosomal-escape-enhancing saponins, different therapeutic nucleic acid such as antisense oligonucleotides for example configured to induce skipping of faulty exons of a wasting muscle cell disorder-associated gene transcript, and advantageous ligands or combinations thereof for targeting said nucleic acids to endocytic receptors on muscle-cells, as well as covalent linkers for connecting the ligands with the nucleic acids and preferably configured for being cleavable under conditions present in human endosomes.

These and other aspects of the disclosure are presented in detail in continuation.

### Definitions

The term “saponin” has its regular established meaning and refers herein to a group of amphipathic glycosides which comprise one or more hydrophilic saccharide chains combined with a lipophilic aglycone core which is termed a sapogenin. The saponin may be naturally occurring or synthetic (i.e. non-naturally occurring). The term “saponin” includes naturally-occurring saponins, functional derivatives of naturally-occurring saponins as well as saponins synthesized de novo through chemical and/or biotechnological synthesis routes. Saponin according to the conjugate of the invention has a triterpene backbone, which is a pentacyclic C30 terpene skeleton, also referred to as sapogenin or aglycone. Within the conjugate of the invention saponin is not considered an effector molecule nor an effector moiety in the conjugates according to the invention. Thus, in the conjugates comprising a saponin and an effector moiety, the effector moiety is a different molecule than the conjugated saponin. In the context of the conjugate of the invention, the term saponin refers to those saponins which exert an endosomal/lysosomal escape enhancing activity, when present in the endosome and/or lysosome of a mammalian cell such as a human cell, towards an effector moiety comprised by the conjugate of the invention and present in said endosome/lysosome together with the saponin.

As used herein, the term “saponin derivative” (also known as “modified saponin”) shall be understood as referring to a compound corresponding to a naturally-occurring saponin (preferably being endosomal/lysosomal escape enhancing activity towards a therapeutic molecule such as nucleic acid, when present together in the endosome or lysosome of a mammalian cell) which has been derivatised by one or more chemical modifications, such as the oxidation of a functional group, the reduction of a functional group and/or the formation of a covalent bond with another molecule (also referred to as “conjugation” or as “covalent conjugation”). Preferred modifications include derivatisation of an aldehyde group of the aglycone core; of a carboxyl group of a saccharide chain or of an acetoxy group of a saccharide chain. Typically, the saponin derivative does not have a natural counterpart, i.e. the saponin derivative is not produced naturally by e.g. plants or trees. The term “saponin derivative” includes derivatives obtained by derivatisation of naturally-occurring saponins as well as derivatives synthesized de novo through chemical and/or biotechnological synthesis routes resulting in a compound corresponding to a naturally-occurring saponin which has been derivatised by one or more chemical modifications. A saponin derivative in the context of the invention should be understood as a saponin functional derivative. “Functional” in the context of the saponin derivative is understood as the capacity or activity of the saponin or the saponin derivative to enhance the endosomal escape of an effector molecule which is contacted with a cell together with the saponin or the saponin derivative.

The term “aglycone core structure” shall be understood as referring to the aglycone core of a saponin without the carbohydrate antennae or saccharide chains (glycans) bound thereto. For example, quillaic acid is the aglycone core structure for SO1861, QS-7 and QS21. Typically, the glycans of a saponin are mono-saccharides or oligo-saccharides, such as linear or branched glycans.

The term “saccharide chain” has its regular scientific meaning and refers to any of a glycan, a carbohydrate antenna, a single saccharide moiety (mono-saccharide) or a chain comprising multiple saccharide moieties (oligosaccharide, polysaccharide). The saccharide chain can consist of only saccharide moieties or may also comprise further moieties such as any one of 4E-Methoxycinnamic acid, 4Z-Methoxycinnamic acid, and 5-O-[5-O-Ara/Api-3,5-dihydroxy-6-methyl-octanoyl]-3,5-dihydroxy-6-methyl-octanoic acid), such as for example present in QS-21.

The term “Api/Xyl-” or “Api- or Xyl-” in the context of the name of a saccharide chain has its regular scientific meaning and here refers to the saccharide chain either comprising an apiose (Api) moiety, or comprising a xylose (Xyl) moiety.

As used herein, the terms “nucleic acid” and “polynucleotide” are synonymous to one another and are to be construed as encompassing any polymeric molecule made of units, wherein a unit comprises a nucleobase (or simply “base” e.g. being a canonical nucleobase like adenine (A), cytosine (C), guanine (G), thymine (T), or uracil (U), or any known non-canonical, modified, or synthetic nucleobase like 5-methylcytosine, 5-hydroxymethylcytosine, xanthine, hypoxanthine, 7-methylguanine; 5,6-dihydrouracil etc.) or a functional equivalent thereof, which renders said polymeric molecule capable of engaging in hydrogen bond-based nucleobase pairing (such as Watson-Crick base pairing) under appropriate hybridisation conditions with naturally-occurring nucleic acids such as deoxyribonucleic acid (DNA) or ribonucleic acid (RNA), which naturally-occurring nucleic acids are to be understood being polymeric molecules made of units being nucleotides.

Hence, from a chemistry perspective, the term nucleic acid under the present definition can be construed as encompassing polymeric molecules that chemically are DNA or RNA, as well as polymeric molecules that are nucleic acid analogues, also known as xeno nucleic acids (XNA) or artificial nucleic acids, which are polymeric molecules wherein one or more (or all) of the units are modified nucleotides or are functional equivalents of nucleotides. Nucleic acid analogues are well known in the art and due to various properties, such as improved specificity and/or affinity, higher binding strength to their target and/or increased stability in vivo, they are extensively used in research and medicine. Typical examples of nucleic acid analogues include but are not limited to locked nucleic acid (LNA) (that is also known as bridged nucleic acid (BNA)), phosphorodiamidate morpholino oligomer (PMO also known as Morpholino), peptide nucleic acid (PNA), glycol nucleic acid (GNA), threose nucleic acid (TNA), hexitol nucleic acid (HNA), 2′-deoxy-2′-fluoroarabinonucleic acid (FANA or FNA), 2′-deoxy-2′-fluororibonucleic acid (2′-F RNA or FRNA); altritol nucleic acids (ANA), cyclohexene nucleic acids (CeNA) etc.

In accordance with the canon, length of a nucleic acid is expressed herein the number of units from which a single strand of a nucleic acid is build. Because each unit corresponds to exactly one nucleobase capable of engaging in one base pairing event, the length is frequently expressed in so called “base pairs” or “bp” regardless of whether the nucleic acid in question is a single stranded (ss) or double stranded (ds) nucleic acid. In naturally-occurring nucleic acids 1 bp corresponds to 1 nucleotide, abbreviated to 1 nt. For example, a single stranded nucleic acid made of 1000 nucleotides (or a double stranded nucleic acid made of two complementary strands each of which is made of 1000 nucleotides) is described as having a length of 1000 base pairs or 1000 bp, which length can also be expressed as 1000 nt or as 1 kilobase that is abbreviated to 1 kb. 2 kilobases or 2 kb are equal to the length of 2000 base pair which equates 2000 nucleotides of a single stranded RNA or DNA. To avoid confusion however, in view of the fact the nucleic acids as defined herein may comprise or consist of units not only chemically being nucleotides but also being functional equivalents thereof, the length of nucleic acids will preferentially be expressed herein in “bp” or “kb” rather than in the equally common in the art denotation “nt”.

In advantageous embodiments as disclosed herein, the nucleic acids are no longer than 1 kb, preferably no longer than 500 bp, most preferably no longer than 250 bp.

In particularly advantageous embodiments, the nucleic acid is an oligonucleotide (or simply an oligo) defined as nucleic acid being no longer than 150 bp, i.e. in accordance with the above provided definition, being any polymeric molecule made of no more than 150 units, wherein each unit comprises a nucleobase or a functional equivalent thereof, which renders said oligonucleotide capable of engaging in hydrogen bond-based nucleobase pairing under appropriate hybridisation conditions with DNA or RNA. Within the ambit of said definition, it will immediately be appreciated that the disclosed herein oligonucleotides can comprise or consist of units not only being nucleotides but also being synthetic equivalents thereof. In other words, from a chemistry perspective, as used herein the term oligonucleotide will be construed as possibly comprising or consisting of RNA, DNA, or a nucleic acid analogue such as but not limited to LNA (BNA), PMO (Morpholino), PNA, GNA, TNA, HNA, FANA, FRNA, ANA, CeNA and/or the like.

As used herein, the term “an endocytic receptor on a muscle cell” is to be understood as referring to surface molecules, likely receptors or transporter that accessible to their specific ligands from the external side or surface of the sarcolemma of the muscle cells and capable of undergoing internalisation via endocytic pathway e.g., upon external stimulation, such as ligand binding to the receptor. In some embodiments, an endocytic receptor on a muscle cell is internalized by clathrin-mediated endocytosis, but can also be internalized by a clathrin-independent pathway, such as, for example, phagocytosis, macropinocytosis, caveolae- and raft-mediated uptake or constitutive clathrin-independent endocytosis. In some embodiments, the endocytic receptor on a muscle cell comprises an intracellular domain, a transmembrane domain, and/or (e.g., and) an extracellular domain, which may optionally further comprise a ligand-binding domain. In some embodiments, the endocytic receptor on a muscle cell becomes internalized by the muscle cell after ligand binding. In some embodiments, a ligand may be a muscle-targeting agent or a muscle-targeting antibody. In some embodiments, an internalizing cell surface receptor is a transferrin receptor (CD71) or for example, CD63 (also known as LAMP-3) belonging to the tetraspanin family.

The term “antibody-oligonucleotide conjugate” or “AOC” has its regular scientific meaning and here refers to any conjugate of an antibody such as an IgG, a Fab, an scFv, an immunoglobulin, an immunoglobulin fragment, one or multiple V_{H} domains, single-domain antibodies, a V_{HH}, a camelid V_{H}, etc., and any polynucleotide (oligonucleotide) molecule that can exert a therapeutic effect when contacted with cells of a subject such as a human patient, such as an oligonucleotide selected from a natural or synthetic string of nucleic acids encompassing DNA, modified DNA, RNA, mRNA, modified RNA, synthetic nucleic acids, presented as a single-stranded molecule or a double-stranded molecule, such as a BNA, an antisense oligonucleotide (ASO, AON), a short or small interfering RNA (siRNA; silencing RNA), an anti-sense DNA, anti-sense RNA, etc.

As used herein, the term “antibody or a binding fragment thereof” refers to a polypeptide that includes at least one immunoglobulin variable domain or at least one antigenic determinant, e.g., paratope that specifically binds to an antigen. In some embodiments, an antibody is a full length antibody. In some embodiments, an antibody is a chimeric antibody. In some embodiments, an antibody is a humanized antibody. However, in some embodiments, an antibody is a Fab fragment, a F(ab′) fragment, a F(ab′)2 fragment, a Fv fragment or a scFv fragment. In some embodiments, an antibody is a nanobody derived from a camelid antibody or a nanobody derived from a shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (a), delta (D), epsilon (e), gamma (g) or mu (m) heavy chain. In some embodiments, the heavy chain of an antibody described herein can comprise a human alpha (a), delta (D), epsilon (e), gamma (g) or mu (m) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (g) heavy chain constant region, such as any known in the art. Non-limiting examples of human constant region sequences have been described in the art, e.g., see U.S. Pat. No. 5,693,780 and Kabat E A et al, (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P, et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

The term “single domain antibody”, or “sdAb”, in short, or ‘nanobody’, has its regular scientific meaning and here refers to an antibody fragment consisting of a single monomeric variable antibody domain, unless referred to as more than one monomeric variable antibody domain such as for example in the context of a bivalent sdAb, which comprises two of such monomeric variable antibody domains in tandem. A bivalent nanobody is a molecule comprising two single domain antibodies targeting epitopes on molecules present at the extracellular side of a cell, such as epitopes on the extracellular domain of a cell surface molecule that is present on the cell. Preferably the cell-surface molecule is a cell-surface receptor. A bivalent nanobody is also named a bivalent single domain antibody. Preferably the two different single domain antibodies are directly covalently bound or covalently bound through an intermediate molecule that is covalently bound to the two different single domain antibodies. Preferably the intermediate molecule of the bivalent nanobody has a molecular weight of less than 10,000 Dalton, more preferably less than 5000 Dalton, even more preferably less than 2000 Dalton, most preferably less than 1500 Dalton.

As used herein, the term “covalently linked” refers to a characteristic of two or more molecules being linked together via at least one covalent bond, i.e. directly, or via a chain of covalent bonds, i.e. via a linker comprising at least one or more atoms.

As used herein, the term “conjugate” is to be construed as a combination of two or more different molecules that have been and are covalently bound. For example, different molecules forming a conjugate as disclosed herein may include one or more nucleic acid or oligonucleotide molecules and one or more ligands that bind to an endocytic receptor present on a surface of a muscle cell, preferably wherein the ligand is an antibody or a binding fragment thereof, such as an IgG, a monoclonal antibody (mAb), a VHH domain or anther nanobody type, a bivalent nanobody molecule comprising two single domain antibodies, etc. In some aspects, the disclosed herein conjugates may be made by covalently linking different molecules via one or more intermediate molecules such as linkers, such as for example via linking to a central or further linker. In a conjugate, not all of the two or more, such as three, different molecules need to be directly covalently bound to each other. Different molecules in the conjugate may also be covalently bound by being both covalently bound to the same intermediate molecule such as a linker or each by being covalently bound to an intermediate molecule such as a further linker or a central linker wherein these two intermediate molecules such as two (different) linkers, are covalently bound to each other. According to this definition even more intermediate molecules, such as linkers, may be present between the two different molecules in the conjugate as long as there is a chain of covalently bound atoms in between.

As used herein, the terms “administering” or “administration” means to provide a complex to a subject in a manner that is physiologically and/or (e.g., and) pharmacologically useful (e.g., to treat a condition in the subject)

As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the embodiments of the invention can operate in other sequences than described or illustrated herein, unless specified otherwise.

The embodiments as described herein can operate in combination and cooperation, unless specified otherwise. Furthermore, the various embodiments, although referred to as “preferred” or “e.g.” or “for example” or “in particular” and the like are to be construed as exemplary manners in which the disclosed herein concepts may be implemented rather than as limiting.

The term “comprising”, used in the claims, should not be interpreted as being restricted to for example the elements or the method steps or the constituents of a compositions listed thereafter; it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present invention, the only enumerated steps of the method are A and B, and further the claim should be interpreted as including equivalents of those method steps. Thus, the scope of the expression “a composition comprising components A and B” should not be limited to a composition consisting only of components A and B, rather with respect to the present invention, the only enumerated components of the composition are A and B, and further the claim should be interpreted as including equivalents of those components.

In addition, reference to an element or a component by the indefinite article “a” or “an” does not exclude the possibility that more than one of the element or component are present, unless the context clearly requires that there is one and only one of the elements or components. The indefinite article “a” or “an” thus usually means “at least one”.

The term “Saponinum album” has its normal meaning and here refers to a mixture of saponins produced by Merck KGaA (Darmstadt, Germany) containing saponins from Gypsophila paniculata and Gypsophila arostii, containing SA1657 and mainly SA1641.

The term “Quillaja saponin” has its normal meaning and here refers to the saponin fraction of Quillaja saponaria and thus the source for all other QS saponins, mainly containing QS-18 and QS-21.

“QS-21” or “QS21” has its regular scientific meaning and here refers to a mixture of QS-21 A-apio (˜63%), QS-21 A-xylo (˜32%), QS-21 B-apio (˜3.3%), and QS-21 B-xylo (˜1.7%).

Similarly, “QS-21A” has its regular scientific meaning and here refers to a mixture of QS-21 A-apio (˜65%) and QS-21 A-xylo (˜35%).

Similarly, “QS-21 B” has its regular scientific meaning and here refers to a mixture of QS-21 B-apio (˜65%) and QS-21 B-xylo (˜35%).

The term “Quil-A” refers to a commercially available semi-purified extract from Quillaja saponaria and contains variable quantities of more than 50 distinct saponins, many of which incorporate the triterpene-trisaccharide substructure Gal-(1→2)-[Xyl-(1→3)]-GlcA- at the C-3beta-OH group found in QS-7, QS-17, QS-18, and QS-21. The saponins found in Quil-A are listed in van Setten (1995), Table 2 [Dirk C. van Setten, Gerrit van de Werken, Gijsbert Zomer and Gideon F. A. Kersten, Glycosyl Compositions and Structural Characteristics of the Potential Immuno-adjuvant Active Saponins in the Quillaja saponaria Molina Extract Quil A, RAPID COMMUNICATIONS IN MASS SPECTROMETRY, VOL. 9, 660-666 (1995)]. Quil-A and also Quillaja saponin are fractions of saponins from Quillaja saponaria and both contain a large variety of different saponins with largely overlapping content. The two fractions differ in their specific composition as the two fractions are gained by different purification procedures.

The term “QS1861” and the term “QS1862” refer to QS-7 and QS-7 api. QS1861 has a molecular mass of 1861 Dalton, QS1862 has a molecular mass of 1862 Dalton. QS1862 is described in Fleck et al. (2019) in Table 1, row no. 28 [Juliane Deise Fleck, Andresa Heemann Betti, Francini Pereira da Silva, Eduardo Artur Troian, Cristina Olivaro, Fernando Ferreira and Simone Gasparin Verza, Saponins from Quillaja saponaria and Quillaja brasiliensis: Particular Chemical Characteristics and Biological Activities, Molecules 2019, 24, 171; doi:10.3390/molecules24010171]. The described structure is the api-variant QS1862 of QS-7. The molecular mass is 1862 Dalton as this mass is the formal mass including proton at the glucuronic acid. At neutral pH, the molecule is deprotonated. When measuring in mass spectrometry in negative ion mode, the measured mass is 1861 Dalton.

The terms “SO1861” and “SO1862” refer to the same saponin of Saponaria officinalis, though in deprotonated form or api form, respectively. The molecular mass is 1862 Dalton as this mass is the formal mass including a proton at the glucuronic acid. At neutral pH, the molecule is deprotonated. When measuring the mass using mass spectrometry in negative ion mode, the measured mass is 1861 Dalton.

### BRIEF DESCRIPTION OF THE DRAWINGS

FIG. 1: Exon skip using (A) DMD-ASO without (left panel) or with (right panel) co-administration of SO1861-EMCH and (B) DMD-PMO without (left panel) or with (right panel) co-administration of SO1861-EMCH in differentiated human myotubes from a non-DMD (healthy) donor (KM155)

FIG. 2: (A) Synthesis of hCD71-PEG4-SPDP precursor to produce (B) hCD71-DMD-ASO and (C) hCD71-DMD-PMO; (D) synthesis of mCD71-SMCC; (E) synthesis of mCD71-M23D

FIG. 3: Exon skip using (A) hCD71-DMD-ASO (DAR2.1) without (left panel) or with (right panel) co-administration of SO1861-EMCH and (B) hCD71-DMD-PMO (DAR3.2) without (left panel) or with (right panel) co-administration of SO1861-EMCH in differentiated human myotubes from a non-DMD (healthy) donor (KM155); see also FIG. 7

FIG. 4: Exon skip using (A) hCD71-DMD-ASO without (left panel) or with (right panel) co-administration of SO1861-EMCH and (B) hCD71-DMD-PMO without (left panel) or with (right panel) co-administration of SO1861-EMCH in differentiated human myotubes from a DMD-affected donor (DM8036); NB, in (A, right panel) the first sample at 0.013 nM (asterisk) shows an empty lane

FIG. 5: Exon skip using mCD71-M23D PMO without (left panel) or with (right panel) co-administration of SO1861-SC-Mal in differentiated murine C2C12 myotubes

FIG. 6: SO1861-EMCH and SO1861-SC-Maleimide, schematic representation.

FIG. 7: Exon skip assessment using (A) hCD71-DMD-ASO (DAR2.2) without (left panel) or with (right panel) co-administration of SO1861-SC-Mal and (B) hCD71-DMD-PMO (DAR3.1) without (left panel) or with (right panel) co-administration of SO1861-SC-Mal in differentiated human myotubes from a non-DMD (healthy) donor (KM155)

FIG. 8: Exon skip assessment using (A) hCD71-DMD-ASO (DAR2.2) without (left panel) or with (right panel) co-administration of SO1861-SC-Mal and (B) hCD71-DMD-PMO (DAR3.1) without (left panel) or with (right panel) co-administration of SO1861-SC-Mal in differentiated human myotubes from a DMD-affected donor (DM8036)

FIG. 9: Exon skip analysis in mice from a single dose mCD71-M23D (group 2) and a vehicle control (group 1) in (A) gastrocnemius, (B) diaphragm, and (C) heart at day 4, day 14, and day 28 post treatment

FIG. 10: (A) Serum creatinine and (B) serum ALT analysis from a single dose study with mCD71-M23D (group 2) and a vehicle control (group 1) at day 4, day 14, day 28

FIG. 11: Synthesis of DBCO-(M23D)₂ via a synthesis scheme involving (A) the synthesis of intermediate 3 (via intermediates 1 and 2); (B) the synthesis of intermediate 4; (C) coupling of intermediate 4 with M23D-SH (reduced form) to achieve the synthesis of intermediate 5; and (D) coupling of intermediates 3 and 5 to yield the desired final product DBCO-(M23D)₂, i.e. a branched scaffold bearing two M23D PMO oligonucleotide payloads.

FIG. 12: Schematic representation of the conjugation procedure for mAb-M23D, such as mCD71-M23D and mCD63-M23D. (A) Preparation of a trimmed and azido modified mAb glycan. (B) Conjugation, via strain-promoted azide-alkyne click reaction, between the trimmed and azido modified mAb glycan and DBCO-(M23D)₂, yielding mAb-(M23D)₄. NB: for clarity of schematic representation, mAb-M23D is shown with DAR 4. (C) Legend explaining symbolically represented glycan residues. (D) Legend explaining symbolically represented molecules.

FIG. 13: Exon 23 skip analysis of (A) mCD71-M23D without (left panel) or with (right panel) co-administration of SO1861-SC-Mal, and (B) mCD63-M23D without (left panel) or with (right panel) co-administration of SO1861-SC-Mal in differentiated murine C2C12 myotubes.

FIG. 14: (A-C) Schematic representation of the conjugation procedure for hAb-DMD-oligo, such as hCD71-5′-SS-DMD-ASO, hCD71-5′-SS-DMD-PMO(1), and hCD71-3′-SS-DMD-PMO(1-5), involving (A) hAb functionalization with PEG4-SPDP at activated lysine (Lys) residues, (B) activation of the protected DMD-oligo, (C) disulfide bond formation between the activated DMD-oligo-SH and hAb-PEG4-SPDP, and (D) figure legend. NB: for clarity of schematic representation, hAb-DMD-oligo is shown with DAR 4.

FIG. 15: Exon 51 skip analysis of (A) hCD71-5′-SS-DMD-ASO (DAR2.1) without (left panel) or with (right panel) co-administration of SO1861-SC-Mal, and (B) hCD71-5′-SS-DMD-PMO(1) (DAR2.2) without (left panel) or with (right panel) co-administration of SO1861-SC-Mal, in differentiated human myotubes from a non-DMD (healthy) donor (KM155).

FIG. 16: Exon 51 skip analysis of (A) hCD71-3′-SS-DMD-PMO(1) (DAR2.1) without (left panel) or with (right panel) co-administration of SO1861-SC-Mal, (B) hCD71-3′-SS-DMD-PMO(2) (DAR3.0) without (left panel) or with (right panel) co-administration of SO1861-SC-Mal, and (C) hCD71-3′-SS-DMD-PMO(3) (DAR2.6) without (left panel) or with (right panel) co-administration of SO1861-SC-Mal, in differentiated human myotubes from a non-DMD (healthy) donor (KM155).

FIG. 17: Exon 53 skip analysis of (A) hCD71-3′-SS-DMD-PMO(4) (DAR2.3) without (left panel) or with (right panel) co-administration of SO1861-SC-Mal, and (B) hCD71-3′-SS-DMD-PMO(5) (DAR2.1) without (left panel) or with (right panel) co-administration of SO1861-SC-Mal, in differentiated human myotubes from a non-DMD (healthy) donor (KM155).

### DETAILED DESCRIPTION

Disclosed herein are improved biologically active pharmaceutical compositions comprising endocytic-escape-enhancing saponins and therapeutic nucleic acids preferably linked to muscle cell-surface endocytic receptor-targeting ligand. The disclosed herein compositions possess the particular advantage of exhibiting the highly desired property of enhanced and effective delivery of therapeutic nucleic acids, such as antisense oligonucleotides, into differentiated muscles cells, striated muscle cells in particular, notably including heart muscle cells.

The innovative concepts presented herein will be described with respect to particular embodiments or aspects of the disclosure, that should be regarded as descriptive and not limiting beyond of what is described in the claims. The particular aspects as described herein can operate in combination and cooperation, unless specified otherwise. While the invention has been described with reference to these embodiments, it is contemplated that alternatives, modifications, permutations and equivalents thereof will become apparent to one having ordinary skill in the art upon reading the specification and upon study of the drawings and graphs. The invention is not limited in any way to the illustrated embodiments. Changes can be made without departing from the scope which is defined by the appended claims.

It is one of several objectives of embodiments of present disclosure is to provide a solution to the problem of inefficient delivery encountered when administering nucleic acid-based therapeutics to human patients suffering from a muscle-wasting disorder and in need of such therapeutics. It a further one of several objectives of the embodiments to provide a solution to the problem of current nucleic acid-based therapies being less efficacious than desired, when administered to human patients in need thereof, due to not being sufficiently capable to reach and/or enter into to the diseased muscle cell with little to no off-target activity on non-diseased cells, when administered to human patients in need thereof.

Without wishing to be bound by any theory, the disclosed herein novel pharmaceutical compositions were conceived based on the observation that a specific group of triterpenoid 12,13-dehydrooleanane-type saponins appears to exhibit potent endosomal-escape enhancing properties for nucleic acid-based therapeutics that are targeted into muscle cells by endocytic-receptor mediated endocytosis.

Endocytic pathways are complex and not fully understood. Nowadays, it is hypothesized that they involve stable compartments connected by vesicular traffic. A compartment is a complex, multifunctional membrane organelle that is specialized for a particular set of essential functions for the cell. Vesicles are considered to be transient organelles, simpler in composition, and are defined as membrane-enclosed containers that form de novo by budding from a pre-existing compartment. In contrast to compartments, vesicles can undergo maturation, which is a physiologically irreversible series of biochemical changes. Early endosomes and late endosomes represent stable compartments in the endocytic pathway while primary endocytic vesicles, phagosomes, multivesicular bodies (also called endosome carrier vesicles), secretory granules, and even lysosomes represent vesicles.

An endocytic vesicle, which arises at the plasma membrane, most prominently from clathrin-coated pits, first fuses with the early endosome, which is a major sorting compartment of approximately pH 6.5. A large part of the internalized cargo and membranes are recycled back to the plasma membrane through recycling vesicles (recycling pathway). Components that should be degraded are transported to the acidic late endosome (pH lower than 6) via multivesicular bodies. Lysosomes are vesicles that can store mature lysosomal enzymes and deliver them to a late endosomal compartment when needed. The resulting organelle is called the hybrid organelle or endolysosome. Lysosomes bud off the hybrid organelle in a process referred to as lysosome reformation. Late endosomes, lysosomes, and hybrid organelles are extremely dynamic organelles, and distinction between them is often difficult. Degradation of the endocytosed molecules occurs inside the endolysosomes.

Endosomal escape is the active or passive release of a substance from the inner lumen of any kind of compartment or vesicle from the endocytic pathway, preferably from clathrin-mediated endocytosis, or recycling pathway into the cytosol. Endosomal escape thus includes but is not limited to release from endosomes, endolysosomes or lysosomes, including their intermediate and hybrid organelles. After entering the cytosol, said substance might move to other cell units such as the nucleus.

As will be demonstrated be the presented herein data, the inclusion of a triterpenoid 12,13-dehydrooleanane-type saponin comprising an aldehyde group at position C-23 of the saponin's aglycone core structure in the therapeutic compositions of the disclosure appeared to stimulate efficient exiting of the therapeutic nucleic acids' from the muscle cells' endosomes into the appropriate muscle cell inner compartments.

In line with these promising observations and findings, in a first general aspect, the invention provides a pharmaceutical composition for use in the treatment or prophylaxis of a muscle wasting disorder, the composition comprising
a nucleic acid, and
a saponin,
   wherein the saponin is a triterpenoid 12,13-dehydrooleanane-type saponin comprising an aldehyde group at position C-23 of the saponin's aglycone core structure under acidic conditions present in endosomes and/or lysosomes of human cells.

That is to say, that in a general aspect, the invention provides a pharmaceutical composition for use in the treatment or prophylaxis of a muscle wasting disorder, the composition comprising
a nucleic acid, and
a saponin,
   wherein the saponin is a triterpenoid 12,13-dehydrooleanane-type saponin comprising at position C-23 of the saponin's aglycone core structure either:
an aldehyde group; or
an acid-sensitive cleavable covalent bond adapted to cleave under acidic conditions such that an aldehyde group at position C-23 of the saponin's aglycone core structure is created or restored upon said cleavage,
   thus being a triterpenoid 12,13-dehydrooleanane-type saponin comprising an aldehyde group at position C-23 of the saponin's aglycone core structure under acidic conditions present in endosomes and/or lysosomes of human cells.

In a next general aspect, the invention also provides a therapeutic combination, for example for a treatment or prophylaxis of a muscle cell-related genetic disorder,
the therapeutic combination comprising:
(a) nucleic acid, and
(b) a saponin
wherein the saponin is a triterpenoid 12,13-dehydrooleanane-type saponin comprising an aldehyde group at position C-23 of the saponin's aglycone core structure under acidic conditions present in endosomes and/or lysosomes of human cells.

Consequently, in a next general aspect, the invention provides a therapeutic combination, preferably for a treatment or prophylaxis of a muscle cell-related genetic disorder,
the therapeutic combination comprising:
(a) nucleic acid, and
(b) a saponin
   wherein the saponin a triterpenoid 12,13-dehydrooleanane-type saponin comprising at position C-23 of the saponin's aglycone core structure either:
an aldehyde group; or
an acid-sensitive cleavable covalent bond adapted to cleave under acidic conditions such that an aldehyde group at position C-23 of the saponin's aglycone core structure is created or restored upon said cleavage,
   thus being a triterpenoid 12,13-dehydrooleanane-type saponin comprising an aldehyde group at position C-23 of the saponin's aglycone core structure under acidic conditions present in endosomes and/or lysosomes of human cells.

As used herein, it will be understood from the context that the nucleic acids forming part of the disclosed herein compositions for the therapeutic purposes and therapeutic combinations are selected such to possess a therapeutic activity for treating or performing prophylaxis of a selected muscle wasting disorder. That is to say, a nucleic acid as comprised in a composition/combination as disclosed herein will be a therapeutic nucleic acid for one disorder, whereas for another disorder it may not cause any benefit. A skilled person aiming to perform a specific treatment of a selected disorder will know how to perform selection of a promising therapeutic nucleic acid and will be able to decide, based by either their knowledge of mutations causing such disorders or based on genetic mutation screening results of a given patient, which therapeutic nucleic acid should be comprised in the novel compositions/combinations as disclosed herein, for performing improved treatment.

In preferred embodiments, composition for the disclosed herein therapeutic or prophylactic use are provided, wherein the muscle wasting disorder is a muscle cell-related genetic disorder, preferably being a congenital myopathy or a muscular dystrophy; preferably wherein the congenital myopathy is selected from nemaline myopathy or congenital fiber-type disproportion myopathy, and/or wherein the muscular dystrophy is selected from a dystrophinopathy, facioscapulohumeral muscular dystrophy, myotonic dystrophy, Emery-Dreifuss muscular dystrophy, limb-girdle muscular dystrophy 1B, congenital muscular dystrophy; or dilated familial cardiomyopathy; most preferably wherein the muscle wasting disorder is a muscle cell-related genetic disorder being a dystrophinopathy, preferably being Duchenne muscular dystrophy.

In a particularly advantageous embodiments, composition for the disclosed herein therapeutic or prophylactic use is provided, wherein the treatment or prophylaxis of the muscle wasting disorder involves antisense therapy, preferably involving exon skipping.

The saponins suitable for application in the disclosed herein compositions and therapeutic combinations have a triterpene 12,13-dehydrooleanane-type backbone wherein the basic structure of the triterpene backbone is a pentacyclic 030 terpene skeleton (also referred to as sapogenin or aglycone) and further comprise an aldehyde group at position C-23 in their native and non-derivative state. Examples of such known saponins are shown in Table 1.

TABLE 1
Saponins displaying (late) endosomal/lysosomal escape enhancing
activity with a aglycone core of the 12,13-dehydrooleanane type⁴⁾

| | Aglycone core | Carbohydrate | Carbohydrate |
|---|---|---|---|
| | with an aldehyde | substituent at | substituent at |
| | group at the C-23 | the C-3beta-OH | the C-28-OH |
| Saponin Name | position | group | group |
| NP-017777 | Gypsogenin | Gal-(1→2)-[Xyl- | Xyl-(1→4)-Rha-(1→2)-[R-(→4)]-Fuc- (R = |
| | | (1→3)]-GlcA- | 4E-Methoxycinnamic acid) |
| NP-017778 | Gypsogenin | Gal-(1→2)-[Xyl- | Xyl-(1→4)-Rha-(1→2)-[R-(→4)]-Fuc- (R = |
| | | (1→3)]-GlcA- | 4Z-Methoxycinnamic acid) |
| NP-017774 | Gypsogenin | Gal-(1→2)-[Xyl- | Xyl-(1→4)-[Gal-(1→3)]-Rha-(1→2)-4- |
| | | (1→3)]-GlcA- | OAc-Fuc- |
| NP-018110^{c}, | Gypsogenin | Gal-(1→2)-[Xyl- | Xyl-(1→4)-[Glc-(1→3)]-Rha-(1→2)-3,4- |
| NP-017772^{d} | | (1→3)]-GlcA- | di-OAc-Fuc- |
| NP-018109 | Gypsogenin | Gal-(1→2)-[Xyl- | Xyl-(1→4)-[Glc-(1→3)]-Rha-(1→2)-[R- |
| | | (1→3)]-GlcA- | (→4)]-3-OAc-Fuc-(R = 4E- |
| | | | Methoxycinnamic acid) |
| NP-017888 | Gypsogenin | Gal-(1→2)-[Xyl- | Glc-(1→3)-Xyl-(1→4)-[Glc-(1→3)]-Rha- |
| | | (1→3)]-GlcA- | (1→2)-4-OAc-Fuc- |
| NP-017889 | Gypsogenin | Gal-(1→2)-[Xyl- | Glc-(1→3)-Xyl-(1→4)-Rha-(1→2)-4-OAc- |
| | | (1→3)]-GlcA- | Fuc- |
| NP-018108 | Gypsogenin | Gal-(1→2)-[Xyl- | Ara/Xyl-(1→3)-Ara/Xyl-(1→4)-Rha/Fuc- |
| | | (1→3)]-GlcA- | (1→2)-[4-OAc-Rha/Fuc-(1→4)]-Rha/Fuc- |
| SA1641^{a}, AE | Gypsogenin | Gal-(1→2)-[Xyl- | Xyl-(1→3)-Xyl-(1→4)-Rha-(1→2)-[Qui- |
| X55^{b} | | (1→3)]-GlcA- | (1→4)]-Fuc- |
| SO1658 | Gypsogenin | Gal-(1→2)-[Xyl- | Glc-(1→3)-[Xyl-(1→3)-Xyl-(1→4)]-Rha- |
| | | (1→3)]-GlcA- | (1→2)-Fuc- |
| gypsoside A⁶⁾ | Gypsogenin | Gal-(1→4)-Glc | Xyl-(1→3)-Fuc-(1→4)-[Xyl-(1→3)-Xyl- |
| | | (1→4)-[Ara- | (1→3)]-Rha- |
| | | (1→3)]-GlcA- | |
| phytolaccagenin | Gypsogenin | absent | absent |
| Gypsophila | Quillaic acid | Gal-(1→2)-[Xyl- | Glc-(1→3)-[Xyl-(1→4)]-Rha-(1→2)-Fuc- |
| saponin 1 | | (1→3)]-GlcA- | |
| (Gyp1) | | | |
| NP-017674 | Quillaic acid | Gal-(1→2)-[Xyl- | Api-(1→3)-Xyl-(1→4)-[Glc-(1→3)]-Rha- |
| | | (1→3)]-GlcA- | (1→2)-Fuc- |
| NP-017810 | Quillaic acid | Gal-(1→2)-[Xyl- | Xyl-(1→4)-[Gal-(1→3)]-Rha-(1→2)-Fuc- |
| | | (1→3)]-GlcA- | |
| AG1 | Quillaic acid | Gal-(1→2)-[Xyl- | Xyl-(1→4)-[Glc-(1→3)]-Rha-(1→2)-Fuc- |
| | | (1→3)]-GlcA- | |
| NP-003881 | Quillaic acid | Gal-(1→2)-[Xyl- | Ara/Xyl-(1→4)-Rha/Fuc-(1→4)-[Glc/Gal- |
| | | (1→3)]-GlcA- | (1→2)]-Fuc- |
| NP-017676 | Quillaic acid | Gal-(1→2)-[Xyl- | Api-(1→3)-Xyl-(1→4)-[Glc-(1→3)]-Rha- |
| | | (1→3)]-GlcA- | (1→2)-[R-(→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| NP-017677 | Quillaic acid | Gal-(1→2)-[Xyl- | Api-(1→3)-Xyl-(1→4)-Rha-(1→2)-[R- |
| | | (1→3)]-GlcA- | (→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| NP-017706 | Quillaic acid | Gal-(1→2)-[Xyl- | Api-(1→3)-Xyl-(1→4)-Rha-(1→2)-[Rha- |
| | | (1→3)]-GlcA- | (1→3)]-4-OAc-Fuc- |
| NP-017705 | Quillaic acid | Gal-(1→2)-[Xyl- | Api-(1→3)-Xyl-(1→4)-[Glc-(1→3)]-Rha- |
| | | (1→3)]-GlcA- | (1→2)-[Rha-(1→3)]-4-OAc-Fuc- |
| NP-017773 | Quillaic acid | Gal-(1→2)-[Xyl- | 6-OAc-Glc-(1→3)-Xyl-(1→4)-Rha-(1→2)- |
| | | (1→3)]-GlcA- | [3-OAc-Rha-(1→3)]-Fuc- |
| NP-017775 | Quillaic acid | Gal-(1→2)-[Xyl- | Glc-(1→3)-Xyl-(1→4)-Rha-(1→2)-[3- |
| | | (1→3)]-GlcA- | OAc--Rha-(1→3)]-Fuc- |
| SA1657 | Quillaic acid | Gal-(1→2)-[Xyl- | Xyl-(1→3)-Xyl-(1→4)-Rha-(1→2)-[Qui- |
| | | (1→3)]-GlcA- | (1→4)]-Fuc- |
| AG2 | Quillaic acid | Gal-(1→2)-[Xyl- | Glc-(1→3)-[Xyl-(1→4)]-Rha-(1→2)-[Qui- |
| | | (1→3)]-GlcA- | (1→4)]-Fuc- |
| GE1741 | Quillaic acid | Gal-(1→2)-[Xyl- | Xyl-(1→3)-Xyl-(1→4)-Rha-(1→2)-[3,4-di- |
| | | (1→3)]-GlcA- | OAc-Qui-(1→4)]-Fuc- |
| SO1542 | Quillaic acid | Gal-(1→2)-[Xyl- | Glc-(1→3)-[Xyl-(1→4)]-Rha-(1→2)-Fuc- |
| | | (1→3)]-GlcA- | |
| SO1584 | Quillaic acid | Gal-(1→2)-[Xyl- | 6-OAc-Glc-(1→3)-[Xyl-(1→4)]-Rha- |
| | | (1→3)]-GlcA- | (1→2)-Fuc- |
| SO1674 | Quillaic acid | Gal-(1→2)-[Xyl- | Glc-(1→3)-[Xyl-(1→3)-Xyl-(1→4)]-Rha- |
| | | (1→3)]-GlcA- | (1→2)-Fuc- |
| SO1700³⁾ | Quillaic acid | Gal-(1→2)-[Xyl- | Xyl-(1→4)-Rha-(1→2)-[Xyl-(1→3)-4- |
| | | (1→3)]-GlcA- | OAc-Qui-(1→4)]-Fuc- |
| Saponarioside | Quillaic acid | Gal-(1→2)-[Xyl- | Xyl-(1→3)-Xyl-(1→4)-Rha-(1→2)-[4- |
| B¹⁾ | | (1→3)]-GlcA- | OAc-Qui-(1→4)]-Fuc- |
| SO1730³⁾ | Quillaic acid | Gal-(1→2)-[Xyl- | Glc-(1→3)-Xyl-(1→4)-Rha-(1→2)-[-4- |
| | | (1→3)]-GlcA- | OAc-Qui-(1→4)]-Fuc- |
| SO1772³⁾ | Quillaic acid | Gal-(1→2)-[Xyl- | 6-OAc-Glc-(1→3)-[Xyl-(1→4)]-Rha- |
| | | (1→3)]-GlcA- | (1→2)-[4-OAc-Qui-(1→4)]-Fuc -- |
| SO1832¹⁾ | Quillaic acid | Gal-(1→2)-[Xyl- | Xyl-(1→3)-Xyl-(1→4)-Rha-(1→2)-[Xyl- |
| (protonated | | (1→3)]-GlcA- | (1→3)-4-OAc-Qui-(1→4)]-Fuc- |
| SO1831) = | | | |
| Saponarioside | | | |
| A | | | |
| SO1861 | Quillaic acid | Gal-(1→2)-[Xyl- | Glc-(1→3)-Xyl-(1→4)-Rha-(1→2)-[Xyl- |
| (deprotonated | | (1→3)]-GlcA- | (1→3)-4-OAc-Qui-(1→4)]-Fuc- |
| SO1862) | | | |
| SO1862 | Quillaic acid | Gal-(1→2)-[Xyl- | Glc-(1→3)-Xyl-(1→4)-Rha-(1→2)-[Xyl- |
| (protonated | | (1→3)]-GlcA- | (1→3)-4-OAc-Qui-(1→4)]-Fuc- |
| SO1861), also | | | |
| referred to as | | | |
| Sapofectosid⁵⁾ | | | |
| SO1904³⁾ | Quillaic acid | Gal-(1→2)-[Xyl- | 6-OAc-Glc-(1→3)-[Xyl-(1→4)]-Rha- |
| | | (1→3)]-GlcA- | (1→2)-[Xyl-(1→3)-4-OAc-Qui-(1→4)]- |
| | | | Fuc- |
| QS-7 (also | Quillaic acid | Gal-(1→2)-[Xyl- | Api/Xyl-(1→3)-Xyl-(1→4)-[Glc-(1→3)]- |
| referred to as | | (1→3)]-GlcA- | Rha-(1→2)-[Rha-(1→3)]-4OAc-Fuc- |
| QS1861) | | | |
| QS-7 api (also | Quillaic acid | Gal-(1→2)-[Xyl- | Api-(1→3)-Xyl-(1→4)-[Glc-(1→3)]-Rha- |
| referred to as | | (1→3)]-GlcA- | (1→2)-[Rha-(1→3)]-4OAc-Fuc- |
| QS1862) | | | |
| QS-17 | Quillaic acid | Gal-(1→2)-[Xyl- | Api/Xyl-(1→3)-Xyl-(1→4)-[Glc-(1→3)]- |
| | | (1→3)]-GlcA- | Rha-(1→2)-[R-(→4)]-Fuc- |
| | | | (R = 5-O-[5-O-Rha-(1→2)-Ara/Api-3,5- |
| | | | dihydroxy-6-methyl-octanoyl]-3,5- |
| | | | dihydroxy-6-methyl-octanoic acid) |
| QS-18 | Quillaic acid | Gal-(1→2)-[Xyl- | Api/Xyl-(1→3)-Xyl-(1→4)-[Glc-(1→3)]- |
| | | (1→3)]-GlcA- | Rha-(1→2)-[R-(→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 A-apio | Quillaic acid | Gal-(1→2)-[Xyl- | Api-(1→3)-Xyl-(1→4)-Rha-(1→2)-[R- |
| | | (1→3)]-GlcA- | (→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 A-xylo | Quillaic acid | Gal-(1→2)-[Xyl- | Xyl-(1→3)-Xyl-(1→4)-Rha-(1→2)-[R- |
| | | (1→3)]-GlcA- | (→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 B-apio | Quillaic acid | Gal-(1→2)-[Xyl- | Api-(1→3)-Xyl-(1→4)-Rha-(1→2)-[R- |
| | | (1→3)]-GlcA- | (→3)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 B-xylo | Quillaic acid | Gal-(1→2)-[Xyl- | Xyl-(1→3)-Xyl-(1→4)-Rha-(1→2)-[R- |
| | | (1→3)]-GlcA- | (→3)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 | Quillaic acid | Gal-(1→2)-[Xyl- | Combination of the carbohydrate chains |
| | | (1→3)]-GlcA- | depicted for QS-21 A-apio, A-xylo, B-apio, |
| | | | B-xylo, for this position at the aglycone |
| | | | (see also the structure depicted as |
| | | | (Scheme Q)) |
| Agrostemmoside | Quillaic acid | Gal-(1→2)-[Xyl- | [4,6-di-OAc-Glc-(1→3)]-[Xyl-(1→4)]-Rha- |
| E (AG1856, | | (1→3)]-GlcA- | (1→2)-[3,4-di-OAc-Qui-(1→4)]-Fuc- |
| AG2.8)²⁾ | | | |

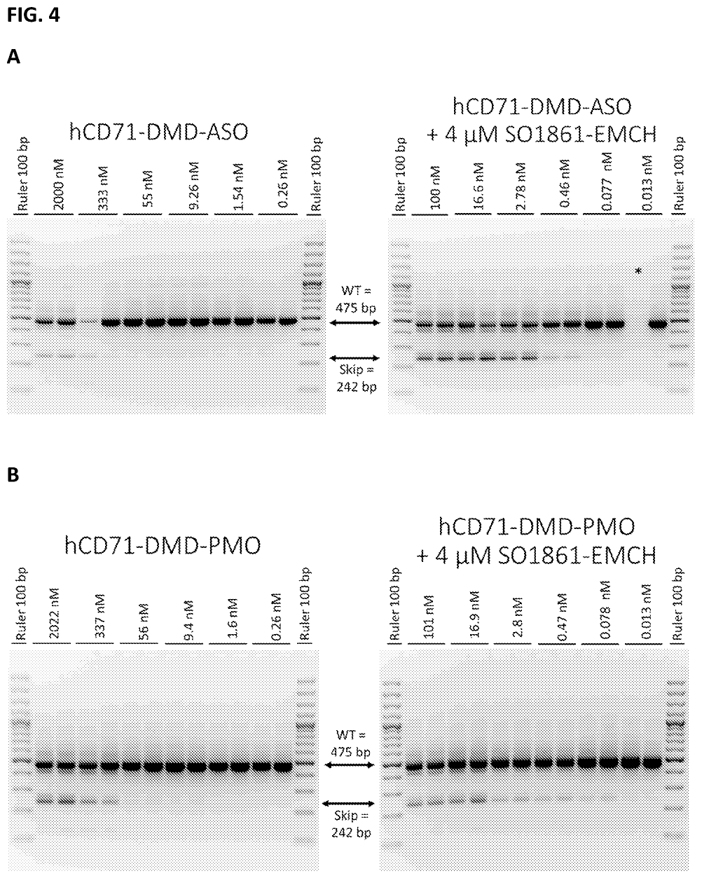

^{a,b}Different names refer to different isolates of the same structure
^{c,d}Different names refer to different isolates of the same structure
¹⁾Jia et al., Major Triterpenoid Saponins from Saponaria officinalis, J. Nat. Prod. 1998, 61, 11, 1368-1373, Publication Date: Sep. 19, 1998, https://doi.org/10.1021/np980167u
²⁾The structure of Agrostemmoside E (also referred to as AG1856 or AG2.8) is given in FIG. 4 of J. Clochard et al, A new acetylated triterpene saponin from Agrostemma githago L. modulates gene delivery efficiently and shows a high cellular tolerance, International Journal of Pharmaceutics, Volume 589, 15 Nov. 2020, 119822.
³⁾Structures of SO1700, SO1730, SO1772, SO1904 are given in Moniuszko-Szajwaj et al., Highly Polar Triterpenoid Saponins from the Roots of Saponaria officinalis L., Helv. Chim. Acta, V99, pp. 347-354, 2016 (doi.org/10.1002/hlca.201500224).
⁴⁾See for example:
thesis by Dr Stefan Böttger (2013): Untersuchungen zur synergistischen Zytotoxizität zwischen Saponinen und Ribosomen inaktivierenden Proteinen Typ I, and
Sama et al., Structure-Activity Relationship of Transfection-Modulating Saponins - A Pursuit for the Optimal Gene Trafficker, Planta Med. Volume 85, pp. 513-518, 2019 (doi: 10.1055/a-0863-4795) and
Fuchs et al., Glycosylated Triterpenoids as Endosomal Escape Enhancers in Targeted Tumor Therapies, Biomedicine, Volume 5, issue 14, 2017 (doi: 10.3390/biomedicines5020014).
⁵⁾Sama et al., Sapofectosid - Ensuring non-toxic and effective DNA and RNA delivery, International Journal of Pharmaceutics, Volume 534, Issues 1-2, 20 Dec. 2017, Pages 195-205 (dx.doi.org/10.1016/j.ijpharm.2017.10.016) & Moniuszko-Szajwaj et al., Highly Polar Triterpenoid Saponins from the Roots of Saponaria officinalis L., Helv. Chim. Acta, V99, pp. 347-354, 2016 (doi.org/10.1002/hlca.201500224).
⁶⁾See for example: doi: 10.1016/s0040-4039(01)90658-6, Tetrahedron Letters No. 8, pp. 477-482, 1963 and pubchem.ncbi.nlm.nih.gov/compound/Gipsoside

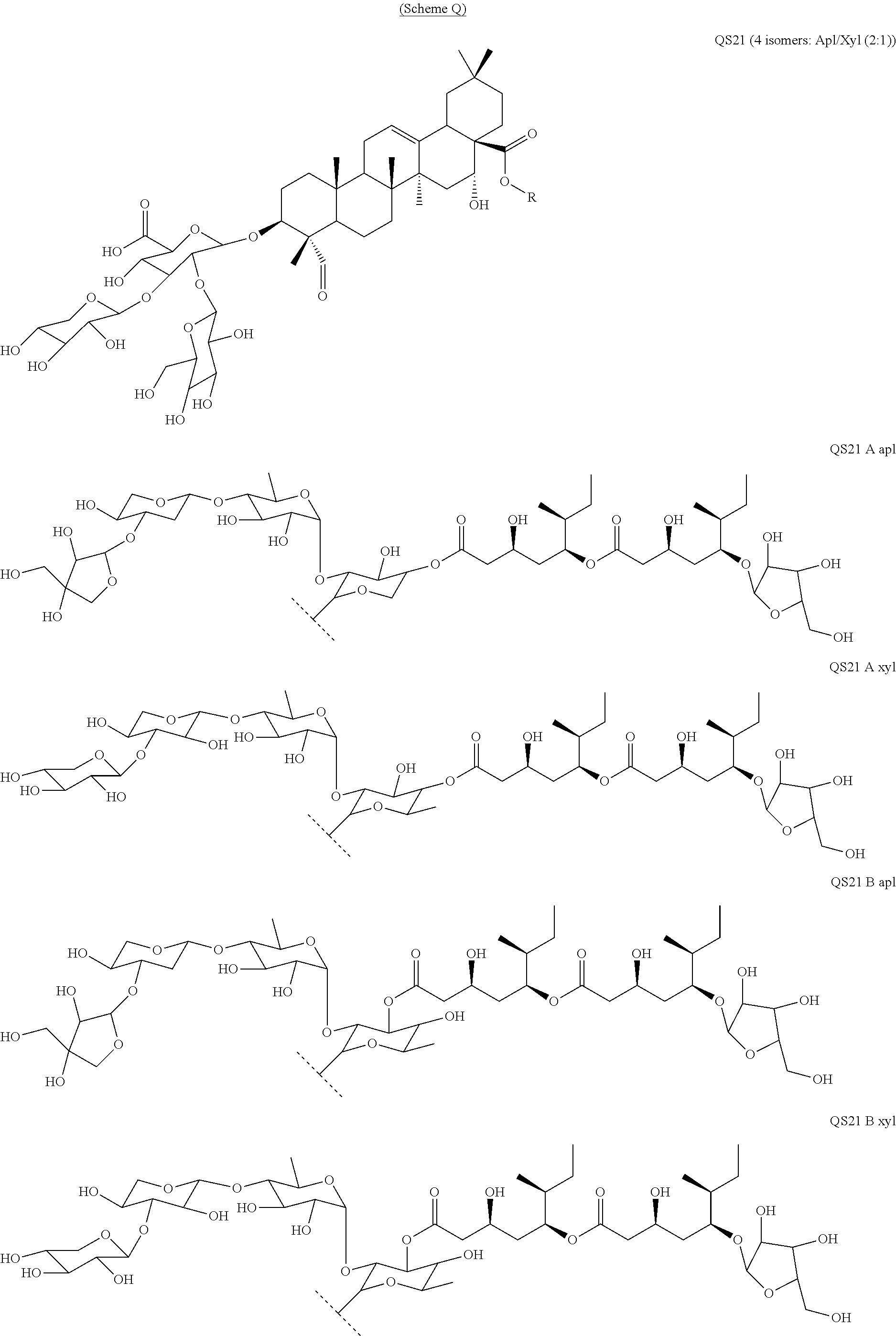

A notable feature of these saponin is the aldehyde group at position C-23 of the saponin's aglycone core structure. Without wishing to be bound by any theory, it was observed that presence of said aldehyde group in the aglycone core structure of the saponin (here, also referred to as ‘aglycone’) is particularly beneficial for the capacity of the saponin to stimulate and/or potentiate the endosomal escape of the therapeutic nucleic acids comprised by the conjugate of the invention.

For the endosomal escape enhancing properties, it is appears to be particularly beneficial that the aldehyde group at position C-23 of the saponin's aglycone core structure in a free aldehyde group once inside of the endosome, and hence any chemical modifications that uncap or restore the free aldehyde group at the position at position C-23 of the saponin's aglycone core structure under acidic conditions present in endosomes and/or lysosomes of human cells can in principle be applied to the disclosed herein suitable saponins shown in their native form in Table 1.

As disclosed herein, such chemical modifications include converting or replacing the aldehyde group at position C-23 into an acid-sensitive cleavable covalent bond adapted to cleave under acidic conditions such that an aldehyde group at position C-23 of the saponin's aglycone core structure is created or restored upon said cleavage, which results in creation of an aldehyde-capped (or aldehyde-shielded) saponin derivative which after entering into the endosomal compartment of a mammalian (e.g. human) cell becomes a saponin comprising the aldehyde group at position C-23 of the saponin's aglycone core structure. For such designed bonds, the restoration of the aldehyde group at position C-23 is a result of the acidity-cause cleavage of said acid-sensitive covalent bond in response to the acidic conditions present in the compartment of mammalian/human endosomes and/or lysosomes.

In line with the above, in a further embodiment, a composition for the disclosed herein therapeutic or prophylactic use or a therapeutic combination is disclosed, wherein the acid-sensitive cleavable covalent bond is selected from any one or more of: a semicarbazone bond, a hydrazone bond, an imine bond, an acetal bond including a 1,3-dioxolane bond, and/or an oxime bond, preferably wherein the acid-sensitive cleavable covalent bond is either a semicarbazone bond or a hydrazone bond

In advantageous embodiments, such cleavable covalent bond can be selected from a semicarbazone bond, a hydrazone bond, or an imine bond.

For example, it was observed that endosomal-escape-enhancing properties of such saponins are still also very pronounced when the aldehyde group is e.g. substituted by a maleimide-comprising moiety attached at said position C-23 with a cleavable covalent bond that cleaves off under acidic conditions present in endosomes and/or lysosomes of human cells, whereby said aldehyde group at position C-23 of the saponin's aglycone core structure is restored upon said cleavage under acidic conditions present in endosomes and/or lysosomes of human cells.

Hence, in a next embodiment, a composition for the disclosed herein therapeutic or prophylactic use or a therapeutic combination is provided, wherein the acid-sensitive cleavable covalent bond attaches a maleimide-comprising moiety to the position C-23 of the saponin's aglycone core structure.

For example, in further possible embodiments, the maleimide-comprising moiety can be part of a molecule comprising or consisting of 4-(6-(2,5-dioxo-2,5-dihydro-1H-pyrrol-1-yl)hexanoyl)piperazine-1-carbohydrazide that is attached at position C-23 of the saponin's aglycone core structure upon forming a semicarbazone bond (further referred to as SC-Maleimide) or wherein the maleimide-comprising moiety is part of a molecule comprising or consisting of N-ε-maleimidocaproic acid hydrazide that is attached at position C-23 of the saponin's aglycone core structure upon forming a hydrazone bond (further referred to as EMCH).

Hence, in a possible embodiment, a composition for the disclosed herein therapeutic or prophylactic use or a therapeutic combination is disclosed, wherein the aldehyde group at position C-23 of the saponin's aglycone core structure is either a free aldehyde group, or is an aldehyde group substituted by a maleimide-comprising moiety attached at said position C-23 with a cleavable covalent bond that cleaves off under acidic conditions present in endosomes and/or lysosomes of human cells, wherein said aldehyde group at position C-23 of the saponin's aglycone core structure is restored upon said cleavage under acidic conditions present in endosomes and/or lysosomes of human cells; preferably wherein the cleavable covalent bond is selected from a semicarbazone bond, a hydrazone bond, or an imine bond; more preferably being selected from a semicarbazone bond and a hydrazone bond; most preferably being a hydrazone bond.

In another embodiment, a composition for the disclosed herein therapeutic or prophylactic use or a therapeutic combination of the disclosure is provided, wherein the maleimide-comprising moiety is part of a molecule comprising or consisting of 4-(6-(2,5-dioxo-2,5-dihydro-1H-pyrrol-1-yl)hexanoyl)piperazine-1-carbohydrazide that is attached at position C-23 of the saponin's aglycone core structure upon forming a semicarbazone bond (further referred to as SC-Maleimide) or wherein the maleimide-comprising moiety is part of a molecule comprising or consisting of N-ε-maleimidocaproic acid hydrazide that is attached at position C-23 of the saponin's aglycone core structure upon forming a hydrazone bond (further referred to as EMCH).

Then, in a further specific embodiment, a composition for the disclosed herein therapeutic or prophylactic use or a therapeutic combination of the disclosure is provided, wherein the saponin is a free saponin defined as consisting of one or more saponin molecules devoid of a direct or indirect covalent conjugation to a macromolecule such as a nucleic acid or a ligand.

In a preferred embodiment, a composition for the disclosed herein therapeutic or prophylactic use or a therapeutic combination of the disclosure is provided, the composition comprising 2-6 μM of the saponin, preferably being 3-5 μM, more preferably being 3.5-4.5 μM, and most being about 4 μM. Most of the known saponins of the 12,13-dehydrooleanane-type that naturally comprise the aldehyde group in position C-23 in their native or unconjugated form are saponins which the aglycone core structure that is either quillaic acid or gypsogenin. An exemplary such saponin is depicted as SAPONIN A and illustrated by the following structure:

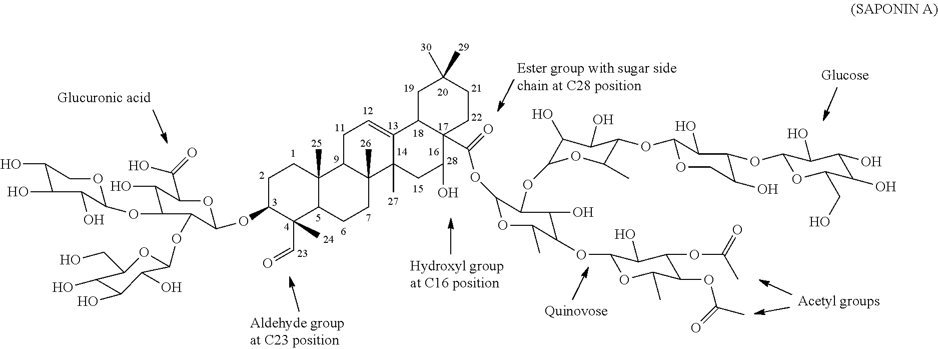

In line with this, it was observed that saponins comprising a quillaic acid aglycone or a gypsogenin aglycone core structure are particularly suitable for the purposes of the present disclosure.

Hence, in a further embodiment, a composition for the disclosed herein therapeutic or prophylactic use or a therapeutic combination of the disclosure is provided, wherein the saponin's aglycone core structure is selected from any one or more of quillaic acid, gypsogenin, and a derivative thereof, preferably wherein the saponin's aglycone core structure is quillaic acid or gypsogenin, more preferably quillaic acid.

In line with the above, a composition for the disclosed herein therapeutic or prophylactic use or a therapeutic combination of the disclosure is provided, wherein wherein the saponin's aglycone core structure is selected from any one or more of
quillaic acid,
quillaic acid derivative wherein the aldehyde group at position C-23 of quillaic acid has been converted to the acid-sensitive cleavable covalent bond at the position C-23 of quillaic acid;
gypsogenin, and
gypsogenin derivative wherein the aldehyde group at position C-23 of gypsogenin has been converted to the acid-sensitive cleavable covalent bond at the position C-23 of gypsogenin;
   preferably wherein the saponin's aglycone core structure is quillaic acid or quillaic acid derivative wherein the aldehyde group at position C-23 of quillaic acid has been converted to the acid-sensitive cleavable covalent bond at the position C-23 of quillaic acid.

Saponins can comprise one or more saccharide chains attached to the aglycone core structure. Preferred saponins of the compositions or therapeutic combinations of the disclosure comprise a single chain (i.e. are monodesmosidic) or two chains (i.e. are bidesmosidic) attached to the triterpene 12,13-dehydrooleanane aglycone core structure comprising an aldehyde group in position C-23.

It was postulated that the sugar chains also play a role in the endosomal-escape-enhancing properties.

In line with the above, in a further embodiment, a composition for the disclosed herein therapeutic or prophylactic use or a therapeutic combination of the disclosure is provided, wherein the saponin is at least a bidesmosidic saponin comprising a first saccharide chain that comprises a terminal glucuronic acid residue and comprises a second saccharide chain that comprises at least four sugar residues in a branched configuration; preferably wherein the first saccharide chain is Gal-(1→2)-[Xyl-(1→3)]-GlcA and/or wherein the branched second saccharide chain of at least four sugar residues comprises a terminal fucose residue and/or a terminal rhamnose residue.

In a related embodiment, a composition for the disclosed herein therapeutic or prophylactic use or a therapeutic combination of the disclosure is provided, wherein the saponin comprises the first saccharide chain at position C-3 of the saponin's aglycone core structure and/or the second saccharide chain at position C-28 of the saponin's aglycone core structure; preferably wherein the first saccharide chain is a carbohydrate substituent at the C-3beta-OH group of the saponin's aglycone core structure and/or wherein the second saccharide chain is a carbohydrate substituent at the C-28-OH group of the saponin's aglycone core structure.

In another embodiment, a composition for the disclosed herein therapeutic or prophylactic use or a therapeutic combination of the disclosure is provided, wherein the saponin is any one or more of:
a) saponin selected from any one or more of list A:
   Quillaja saponaria saponin mixture, or a saponin isolated from Quillaja saponaria, for example Quil-A, QS-17-api, QS-17-xyl, QS-21, QS-21A, QS-21B, QS-7-xyl;
   Saponinum album saponin mixture, or a saponin isolated from Saponinum album;
   Saponaria officinalis saponin mixture, or a saponin isolated from Saponaria officinalis; and
   Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B: SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C:
   AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS-7, QS-7 api, QS-17, QS-18, QS-21 A-apio, QS-21 A-xylo, QS-21 B-apio and QS-21 B-xylo;
      preferably, the saponin is any one or more of a saponin selected from list B or C, more preferably, a saponin selected from list C.

In a particular embodiment, a composition for the disclosed herein therapeutic or prophylactic use or a therapeutic combination of the disclosure is provided, wherein the saponin is any one or more of AG1856, GE1741, a saponin isolated from Quillaja saponaria, Quil-A, QS-17, QS-21, QS-7, SA1641, a saponin isolated from Saponaria officinalis, Saponarioside B, SO1542, SO1584, SO1658, SO1674, SO1700, SO1730, SO1772, SO1832, SO1861, SO1862 and SO1904; preferably wherein the saponin is any one or more of QS-21, SO1832, AG1856, SO1861, SA1641 and GE1741; more preferably wherein the saponin is QS-21, SO1832 or SO1861; most preferably being SO1861.

In a more particular embodiment, a composition for the disclosed herein therapeutic or prophylactic use or a therapeutic combination of the disclosure is provided, wherein the saponin is a saponin isolated from Saponaria officinalis, preferably wherein the saponin is any one or more of Saponarioside B, SO1542, SO1584, SO1658, SO1674, SO1700, SO1730, SO1772, SO1832, SO1861, SO1862 and SO1904; more preferably wherein the saponin is any one or more of SO1542, SO1584, SO1658, SO1674, SO1700, SO1730, SO1772, SO1832, SO1861, SO1862 and SO1904, even more preferably wherein the saponin is any one or more of SO1832, SO1861 and SO1862; even more preferably wherein the saponin is SO1832 and SO1861; most preferably being SO1861.

In further particular embodiments, compositions or therapeutic combinations as disclosed herein can be prepared with the suitable triterpenoid 12,13-dehydrooleanane-type saponins comprising an aldehyde group at position C-23 of the saponin's aglycone core structure, wherein one or more, preferably one of:
i. an aldehyde group in the aglycone core structure of the at least one saponin has been derivatised when present,
ii. a carboxyl group of a glucuronic acid moiety in a first saccharide chain of the at least one saponin has been derivatised when present in the at least one saponin, and
iii. at least one acetoxy (Me(CO)O—) group in a second saccharide chain of the at least one saponin has been derivatised if present.

In more particular embodiments, compositions or therapeutic combinations as disclosed can be provided wherein the at least one triterpenoid 12,13-dehydrooleanane-type saponins comprising an aldehyde group at position C-23 of the saponin's aglycone core structure further comprises:
i. an aglycone core structure comprising an aldehyde group which has been derivatised by:
   reduction to an alcohol;
   transformation into a hydrazone bond through reaction with N-ε-maleimidocaproic acid hydrazide (EMCH) wherein the maleimide group of the EMCH is optionally derivatised by formation of a thioether bond with mercaptoethanol;
   transformation into a hydrazone bond through reaction with N-[β-maleimidopropionic acid]hydrazide (BMPH) wherein the maleimide group of the BMPH is optionally derivatised by formation of a thioether bond with mercaptoethanol; or
   transformation into a hydrazone bond through reaction with N-[κ-maleimidoundecanoic acid]hydrazide (KMUH) wherein the maleimide group of the KMUH is optionally derivatised by formation of a thioether bond with mercaptoethanol; or
ii. a first saccharide chain comprising a carboxyl group, preferably a carboxyl group of a glucuronic acid moiety, which has been derivatised by transformation into an amide bond through reaction with 2-amino-2-methyl-1,3-propanediol (AMPD) or N-(2-aminoethyl)maleimide (AEM); or
iii. a second saccharide chain comprising an acetoxy group (Me(CO)O—) which has been derivatised by transformation into a hydroxyl group (HO—) by deacetylation; or
iv. any combination of two or three derivatisations i., ii. and/or iii., preferably any combination of two derivatisations of i., ii. and iii.

In a specific embodiment, provided are compositions and therapeutic combinations, wherein the at least one saponin comprises a first saccharide chain and a second saccharide chain, wherein the first saccharide chain comprises more than one saccharide moiety and the second saccharide chain comprises more than one saccharide moiety, and wherein the aglycone core structure is quillaic acid or gypsogenin, more preferably is quillaic acid, wherein one, two or three, preferably one or two, of:
i. an aldehyde group in the aglycone core structure has been derivatised,
ii. a carboxyl group of a glucuronic acid moiety in the first saccharide chain has been derivatised, and
iii. at least one acetoxy (Me(CO)O—) group in the second saccharide chain has been derivatised.

An embodiment is the conjugate of the invention, wherein one, two or three, preferably one or two, more preferably one, of:
iv. an aldehyde group in the aglycone core structure of the at least one saponin has been derivatised when present,
v. a carboxyl group of a glucuronic acid moiety in a first saccharide chain of the at least one saponin has been derivatised when present in the at least one saponin, and at least one acetoxy (Me(CO)O—) group in a second saccharide chain of the at least one saponin has been derivatised if present.

In a specific embodiment, a composition or a therapeutic combination according to the disclosure is provided wherein the aldehyde function in position C-23 of the aglycone core structure of the saponin is covalently bound to a EMCH cap. Binding of the EMCH to the aldehyde group of the aglycone of the saponin results in formation of a hydrazone bond. Such a hydrazone bond is a typical example of a cleavable bond that restores the aldehyde group at position C-23 of the saponin's aglycone core structure under the acidic conditions inside endosomes and lysosomes.

The development of the presented herein advantageous compositions was based on the surprising realisation that, thanks to the inclusion of the endosomal-escape-enhancing saponin in the compositions of the invention, nucleic acids can be delivered with an improved efficiently into muscle cells to aid the treatment and/or prophylaxis of muscle-wasting disorders.

In line with this, in an advantageous embodiment, the nucleic acid is a therapeutic nucleic acid adapted to target mutated transcript or a genomic mutation within a gene affected in a particular muscle cell-related genetic disorder. A list of such potentially targetable genetic targets and muscle cell-related genetic disorder associated therewith can be for instance found in Cardamone M, et al., 2008.

In a particular embodiment, such genetic target is the mutated human dystrophin transcript which expression causes dystrophinopathies such as DMD, for which the proof-of-concepts experiments demonstrating the potential of the disclosed herein compositions are presented in the continuation. However, other mutations in known genes can also be targeted by antisense therapy, such as the ones in but not limited to: DUX4/double homeobox 4 underling facioscapulohumeral muscular dystrophy, or DMPK underlying myotonic dystrophy type 1, or EMD/emerin and LMNA/lamin A/C underling the Emery-Dreifuss muscular dystrophy, or MYOT/myotilin, LMNA/lamin A/C underling limb-girdle muscular dystrophy 1. Further examples LAMA2/merosin or laminin-α2 chain/any of COL6A genes encoding for collagen 6A that become mutated in congenital muscular dystrophy or LMNA/lamin A/C in dilated familial cardiomyopathy. Further mutations that can be targeted by nuclei acids present in the disclosed herein conjugates and compositions can be found in genes like NEB/nebulin, ACTA/skeletal muscle alpha-actin, TPM3/alpha-tropomyosin-3, TPM2/beta-tropomyosin-2, TNNT1/troponin T1, LMOD3/leiomodin-3, MYPN/myopalladin etc. (nemalin myopathy) or TPM3/alpha-tropomyosin-3, CTA/skeletal muscle alpha-actin, RYR1/ryanodine receptor channel (congenital fibre-type disproportion myopathy) or advantageously in TTN gene (titin).

In a possible embodiment, a composition for the disclosed herein therapeutic or prophylactic use or a therapeutic combination of the disclosure is provided, wherein the nucleic acid comprises or consists of any one of the following: morpholino phosphorodiamidate oligomer (PMO), 2′-O-methyl (2′-OMe) phosphorothioate RNA, 2′-O-methoxyethyl (2′-O-MOE) RNA {2′-O-methoxyethyl-RNA (MOE)}, locked or bridged nucleic acid (BNA), 2′-0,4′-aminoethylene bridged nucleic acid (BNANC), peptide nucleic acid (PNA), 2′-deoxy-2′-fluoroarabino nucleic acid (FANA), 3′-fluoro hexitol nucleic acid (FHNA), glycol nucleic acid (GNA), threose nucleic acid (TNA), silencing RNA (siRNA), short hairpin RNA (shRNA), microRNA (miRNA), antagomir (miRNA antagonists), aptamer RNA or aptamer DNA, single-stranded RNA or single-stranded DNA, double-stranded RNA (dsRNA) or double-stranded DNA; preferably wherein the nucleic acidoligonucleotide comprises or consists of a morpholino phosphorodiamidate oligomer (PMO) or a 2′-O-methyl (2′-OMe) phosphorothioate RNA.

In a further embodiment, a composition for the disclosed herein therapeutic or prophylactic use or a therapeutic combination is provided, wherein wherein the nucleic acid is designed to induce exon skipping of the human dystrophin gene transcript, preferably wherein the exon skipping involves exon 51 skipping or exon 53 skipping or exon 45 skipping;

more preferably wherein the nucleic acid is a 2′O-methyl-phosporothioate antisense oligonucleotide or a phosphorodiamidate morpholino oligomer antisense oligonucleotide that is designed to induce the exon 51 skipping or the exon 53 skipping or the exon 45 skipping,
even more preferably wherein the nucleic acid is selected from eteplirsen, drisapersen, golodirsen, viltolarsen, and casimersen.

In a particularly advantageous embodiment, a composition for the disclosed herein therapeutic or prophylactic use or a therapeutic combination of the disclosure is provided, wherein the nucleic acid is an oligonucleotide defined as a nucleic acid that is no longer than 150 nt, preferably wherein the oligonucleotide has a size of 5-150 nt, preferably being 8-100 nt, most preferably being 10-50 nt. Preferably, the oligonucleotide is an antisense oligonucleotide, even more preferably being a mutation specific antisense oligonucleotide, most preferably being an oligonucleotide designed to induce exon skipping.

As used herein the term oligonucleotide shall be understood as encompassing both the oligomers that are made of naturally occurring nucleotides and hence, chemically are oligonucleotides, as well as oligomers comprising modified oligonucleotides or analogues thereof. For example, a synthetic oligomer may comprise e.g. 2′ modified nucleosides which can be selected from: 2′-fluoro (2′-F), 2′-O-methyl (2′-O-Me), 2′-O-methoxyethyl (2′-MOE). 2′-O-aminopropyl (2′O-AP), 2′-O-dimethylaminoethyl (2′-O-DMAOE), 2′-O-dimethylaminopropyl (2′-O-DMAP),2′-O-dimethylaminoethyloxyethyl (2′-O-DMAEOE), 2′-O—N-methylacetamido (2′-O-NMA), locked nucleic acid (LNA), ethylene-bridged nucleic acid (ENA), and (S)-constrained ethylbridged nucleic acid (cEt), etc. In line with this, in a possible embodiment, the oligonucleotide can structurally or functionally be defined as any of: a deoxyribonucleic acid (DNA) oligomer, ribonucleic acid (RNA) oligomer, anti-sense oligonucleotide (ASO, AON), short interfering RNA (siRNA), anti-microRNA (anti-miRNA), DNA aptamer, RNA aptamer, mRNA, mini-circle DNA, peptide nucleic acid (PNA), phosphoramidate morpholino oligomer (PMO), locked nucleic acid (LNA), bridged nucleic acid (BNA), 2′-deoxy-2′-fluoroarabino nucleic acid (FANA), 2′-O-methoxyethyl-RNA (MOE), 3′-fluoro hexitol nucleic acid (FHNA), glycol nucleic acid (GNA), threose nucleic acid (TNA), BNA-based siRNA, and BNA-based antisense oligonucleotide (BNA-AON), an siRNA, such as BNA-based siRNA, selected from chemically modified siRNA, metabolically stable siRNA and chemically modified, metabolically stable siRNA, or any other category known in the art.

From functional perspective, in advantageous embodiments, a composition for the disclosed herein therapeutic or prophylactic use or a therapeutic combination of the disclosure is provided, wherein the oligonucleotide is an antisense oligonucleotide, preferably being a mutation specific antisense oligonucleotide, most preferably being an oligonucleotide designed to induce exon skipping.

In a related embodiment, a composition for the disclosed herein therapeutic or prophylactic use or a therapeutic combination of the disclosure is provided, wherein the oligonucleotide comprises or consists of any one of the following: morpholino phosphorodiamidate oligomer (PMO), 2′-O-methyl (2′-OMe) phosphorothioate RNA, 2′-O-methoxyethyl (2′-O-MOE) RNA {2′-O-methoxyethyl-RNA (MOE)}, locked or bridged nucleic acid (BNA), 2′-O,4′-aminoethylene bridged nucleic acid (BNANC), peptide nucleic acid (PNA), 2′-deoxy-2′-fluoroarabino nucleic acid (FANA), 3′-fluoro hexitol nucleic acid (FHNA), glycol nucleic acid (GNA), threose nucleic acid (TNA), silencing RNA (siRNA), short hairpin RNA (shRNA), microRNA (miRNA), antagomir (miRNA antagonists), aptamer RNA or aptamer DNA, single-stranded RNA or single-stranded DNA, double-stranded RNA (dsRNA) or double-stranded DNA.

In particularly preferred embodiments, composition for the disclosed herein therapeutic or prophylactic use or a conjugate according to the disclosure is provided, wherein the oligonucleotide comprises or consists of a morpholino phosphorodiamidate oligomer (PMO) or a 2′-O-methyl (2′-OMe) phosphorothioate RNA.

In a particular and advantageous for DMD-specific embodiment, a composition for the disclosed herein therapeutic or prophylactic use or a therapeutic combination of the disclosure is provided, wherein the oligonucleotide is designed to induce exon skipping of the human dystrophin gene transcript, preferably wherein the exon skipping involves exon 51 skipping or exon 53 skipping or exon 45 skipping; more preferably wherein the oligonucleotide is a 2′O-methyl-phosporothioate antisense oligonucleotide or a phosphorodiamidate morpholino oligomer antisense oligonucleotide that is designed to induce the exon 51 skipping or the exon 53 skipping or the exon 45 skipping,

In a very particular embodiment in line with the directly-preceding one, the oligonucleotide is selected from eteplirsen, drisapersen, golodirsen, viltolarsen, and casimersen.

In an advantageous embodiment, a composition for the disclosed herein therapeutic or prophylactic use or a therapeutic combination of the disclosure is provided, wherein the composition comprises two or more different nucleic acids, the two or more different nucleic acids preferably being two or more different oligonucleotides, more preferably wherein at least one of the two or more different oligonucleotides is an antisense oligonucleotide.

Such combinations of two or more therapeutic nucleic acids are known in the art and for muscle-wasting disorders e.g. a combined approach based on two AONs for dual exon skipping in myostatin and dystrophin was proposed for the management of Duchenne muscular dystrophy [Kemaladewi et al, 2011].

In a particularly preferred embodiment, a composition for the disclosed herein therapeutic or prophylactic use or a therapeutic combination of the disclosure is provided, wherein the nucleic acid is conjugated with a ligand of an endocytic receptor on a muscle cell.

With regard to the ligand of an endocytic receptor on a muscle cell, it should be noted that many endocytic receptors expressed on the surface of muscle cells are known and some of which like the transferrin receptor (CD71) or muscle-specific kinase (MuSK) are described in WO2018129384 or WO2020028857. Further examples of suitable receptors may include muscle-transmembrane transporters, for instance GLUT4 or ENT2 (described with many others in Ebner, 2015), or for example tetraspanin CD63 [Baik, 2021]. In fact, a lot of endocytic receptors present on the surface of muscle cells have been characterised so far, with the transferrin receptor (CD71) and perhaps insulin-like growth factor 1 (IGF-I) receptor (IGF1R) being the most investigated ones. The ligands for these receptors can be selected from natural ligands, such as transferrin (To being a natural ligand of CD71, or LDL being a ligand of the LDL receptor. Alternatively, they can be non-naturally occurring ligands such as various types of antibodies or binding fragments thereof, or alternatively can be synthetic ligands like zymozan A that binds to endocytic mannose receptors, or synthetic fragments of naturally existing ligands such as fragments of IGF-I being a ligand of IGF1R or fragments of IGF-II being a ligand of CI-MPR (also known as IGF2R).

In a preferred embodiment, a for the disclosed herein therapeutic or prophylactic use or a therapeutic combination of the disclosure is provided, wherein the endocytic receptor on a muscle cell to which the ligand, that conjugated with the nucleic acid, binds is selected from: transferrin receptor (CD71), insulin-like growth factor 1 (IGF-1) receptor (IGF1R), tetraspanin CD63; muscle-specific kinase (MuSK), glucose transporter GLUT4, cation independent mannose 6 phosphate receptor (CI-MPR).

In a further preferred embodiment, a composition for the disclosed herein therapeutic or prophylactic use or a therapeutic combination of the disclosure is provided, wherein the ligand, that is conjugated with the nucleic acid, is selected from any one of:
insulin-like growth factor 1 (IGF-I) or a fragment thereof;
insulin-like growth factor 2 (IGF-II) or a fragment thereof
Mannose 6 phosphate
transferrin (Tf),
zymozan A, and
an antibody or a binding fragment thereof specific for binding to the endocytic receptor, wherein the endocytic receptor is preferably selected from: transferrin receptor (CD71), insulin-like growth factor 1 (IGF-I) receptor (IGF-IR), tetraspanin CD63, muscle-specific kinase (MuSK), glucose transporter GLUT4, cation independent mannose 6 phosphate receptor (CI-MPR), and LDL receptor;
   preferably wherein the ligand is an antibody or a binding fragment thereof that is specific for binding to a transferrin receptor, more preferably wherein the ligand is a monoclonal antibody or a Fab′ fragment or at least one single domain antibody specific for binding to a transferrin receptor, even more preferably wherein the ligand is a monoclonal antibody specific for binding to a transferrin receptor.

In a related particularly advantageous embodiment, the ligand, that is conjugated with the nucleic acid, is a monoclonal antibody or a Fab′ fragment or at least one single domain antibody specific for binding to a transferrin receptor, even more preferably wherein the ligand is a monoclonal antibody specific for binding to a transferrin receptor.

In a further advantageous embodiment, the ligand, that is conjugated with the nucleic acid, is a monoclonal antibody such as a humanized or a human monoclonal antibody, an IgG, a molecule comprising or consisting of a single-domain antibody, at least one V_{HH} domain, preferable a camelid V_{H}, a variable heavy chain new antigen receptor (VNAR) domain, a Fab, an scFv, an Fv, a dAb, an F(ab)2 and a Fcab fragment.

In certain advantageous embodiments, a composition for the disclosed herein therapeutic or prophylactic use or a therapeutic combination of the disclosure is provided, wherein the ligand is conjugated with 2-5 molecules of the nucleic acid per 1 molecule of the ligand; preferably being 3-4 molecules of the nucleic acid per 1 molecule of the ligand; more preferably wherein the ligand is on average conjugated with 4 molecules of the nucleic acid per 1 molecule of the ligand.

In an embodiment, a composition for the disclosed herein therapeutic or prophylactic use or a therapeutic combination of the disclosure is provided, wherein the ligand comprises a chain of amino acid residues comprising at least one cysteine residue and/or at least one lysine residue and wherein the covalent linking of the nucleic acid with the ligand comprises a covalent bond with at least one cysteine residue and/or at least one lysine residue;
preferably wherein more than one molecule of the nucleic acid is linked to one molecule of the ligand via a separate cysteine residue and/or a separate lysine residue;
more preferably wherein the ligand comprises a chain of amino acid residues comprising a multicysteine repeat, possibly being a tetracysteine repeat represented by the sequence HRWCCPGCCKTF (SEQ ID NO.4), and wherein the covalent linking of the nucleic acid with the ligand comprises a covalent bond with any one or more of the cysteine residues of the multicysteine repeat;
most preferably wherein more than one molecule of the nucleic acid is linked to one molecule of the ligand via a separate cysteine residue of the multicysteine repeat.

In a related embodiment, a composition for the disclosed herein therapeutic or prophylactic use or a therapeutic combination of the disclosure is provided, wherein the covalent linking of the nucleic acid with the ligand is made via a linker to which the nucleic acid is covalently bound;
preferably wherein the linker comprises or consists of linker succinimidyl 3-(2-pyridyldithio)propionate (SPDP); possibly wherein the linker covalently links the nucleic acid to a lysine residue of the ligand, or to a glycan residue, preferably a partially-trimmed glycan.

In certain applications it can be advantageous if the one or more of the nucleic acid molecules, e.g. oligonucleotide molecules, are bound to the ligand via a cleavable bond, wherein the cleavable bond is subject to cleavage under for example acidic, reductive, enzymatic and/or light-induced conditions. The cleavable bond being subject to cleavage under acidic conditions present in endosomes and/or lysosomes of human cells is preferred.

In line with the above, in a related and advantageous embodiment, a composition for the disclosed herein therapeutic or prophylactic use or a therapeutic combination of the disclosure is provided, wherein the linker is a cleavable linker subject to cleavage under acidic, reductive, enzymatic and/or light-induced conditions;
preferably wherein the linker comprises a cleavable bond selected from:
   a bond subject to cleavage under acidic conditions such as a semicarbazone bond, a hydrazone bond, an imine bond, an acetal bond including a 1,3-dioxolane bond, a ketal bond, an ester bond, and/or an oxime bond,
   a bond susceptible to proteolysis, for example amide or peptide bond, preferably subject to proteolysis by Cathepsin B;
   a red/ox-cleavable bond such as a disulfide bond, or a thiol-exchange reaction-susceptible bond such as a thio-ether bond;
   more preferably wherein the linker comprises a cleavable bond selected from:
   a bond subject to cleavage under acidic conditions such as a semicarbazone bond, a hydrazone bond or an imine bond, and/or
   a bond susceptible to proteolysis, for example proteolysis by Cathepsin B, and/or
   a bond susceptible for cleavage under reductive conditions such as a disulfide bond;

Advantageously, the bond is an acid-sensitive bond, i.e. subject to cleavage in vivo under acidic conditions present in endosomes and/or lysosomes of human cells, preferably of pH≤6.5, preferably pH≤6, more preferably pH≤5.5, more preferably being an acid-sensitive bond selected from a semicarbazone bond and a hydrazone bond; most preferably being a hydrazone bond.

It is preferred that such a cleavable bond is not susceptible, or only to a minor extent, to cleavage when the nucleic acid-ligand conjugate is present outside the endosome and lysosome of the cell, such as outside the cell or in the endocytosed vesicle after the conjugate engaged with an endocytic receptor by binding of the ligand to its target endocytic receptor on a muscle cell. For example, the cleavable bond is preferably less susceptible to cleavage when the conjugate is present in the circulation of a human subject and/or is present extracellularly in an organ of the human subject, compared to the susceptibility for cleavage of the bond when the conjugate is in the endosome or in the lysosome of a target cell, herein being the muscle cells.

In a particular embodiment, a composition for the disclosed herein therapeutic or prophylactic use or a conjugate according to the disclosure is provided, wherein the linker is directly or indirectly covalently linked to the ligand.

In a further particular embodiment, a composition for the disclosed herein therapeutic or prophylactic use or a therapeutic combination of the disclosure is provided, wherein the saponin is or comprises at least one molecule of any of SO1861, SO1861-EMCH, or SO1861-SC-Maleimide, preferably SO1861-EMCH or SO1861-SC-Maleimide, the nucleic acid is drisapersen or eteplirsen, and the ligand conjugated with the nucleic acid is antiCD71 antibody or a binding fragment thereof. For the disclosed herein therapeutic or prophylactic use, the use is in intravenous or subcutaneous or intramuscular administration to a human subject, preferably being intramuscular administration.

In another embodiment, a composition for the disclosed herein therapeutic or prophylactic use, the use is in intravenous or subcutaneous or intramuscular administration to a human subject, preferably being intramuscular administration.

In a yet another embodiment, a composition for the disclosed herein therapeutic or prophylactic use or a therapeutic combination of the disclosure is provided, comprising a pharmaceutically acceptable excipient and/or pharmaceutically acceptable diluent.

In a related aspect, further provided is a kit comprising the components (a) and (b) of the therapeutic combination, possibly wherein the components (a) and (b) are in separate vials, preferably wherein the components (a) and (b) are provided in a mixture suitable form subcutaneous or intramuscular injection.

Last but not least, in a particular embodiment the therapeutic combinations and/or kits according to the disclosure are provided for use as a medicament.

### EXAMPLES

SO1861 was isolated and purified by by Extrasynthese, France and/or Analyticon Discovery GmbH, Germany from raw plant extract obtained from Saponaria officinalis L.

An antisense oligonucleotide with the sequence 5′-UCAAGGAAGAUGGCAUUUCU-3′ [SEQ ID NO: 1], and an ASO with the same sequence and a thiol modification (DMD-ASO and 5′-thiol-DMD-ASO, respectively) were custom-made and purchased from Hanugen Therapeutics Pvt Ltd. A PMO with the sequence 5′-CTCCAACATCAAGGAAGATGGCATTTCTAG-3′ (DMD-PMO or DMD-PMO(1)) [SEQ ID NO: 2], and a PMO with the same sequence with a disulfide amide modification (5′-disulfidamide-DMD-PMO or 5′-disulfideamide-DMD-PMO(1)), were custom-made and purchased from Gene Tools, LLC. A PMO with the sequence 5′-CTCCAACATCAAGGAAGATGGCATTTCTAG-3′ (DMD-PMO(1)) [SEQ ID NO: 2] and a disulfide amide modification on the 3′ (3′-disulfidamide-DMD-PMO(1)) was custom-made and purchased from Gene Tools. A PMO with the sequence 5′-GGCCAAACCTCGGCTTACCTGAAAT-3′ (M23D) [SEQ ID NO: 3], and a PMO with the same sequence with a disulfide amide modification (3′-disulfideamide-M23D) were custom-made and purchased from Gene Tools, LLC. A PMO with the sequence 5′-GTGTCACCAGAGTAACAGTCTGAGTAGGAG-3′ (DMD-PMO(2)) [SEQ ID NO: 16] and a disulfide amide modification on the 3′ (3′-disulfidamide-DMD-PMO(2)) was custom-made and purchased from Gene Tools. A PMO with the sequence 5′-GGCAGTTTCCTTAGTAACCACAGGTTGTGT-3′ (DMD-PMO(3)) [SEQ ID NO: 17] and a disulfide amide modification on the 3′ (3′-disulfidamide-DMD-PMO(3)) was custom-made and purchased from Gene Tools. A PMO with the sequence 5′-GTTGCCTCCGGTTCTGAAGGTGTTC-3′ (DMD-PMO(4)) [SEQ ID NO: 18] and a disulfide amide modification on the 3′ (3′-disulfidamide-DMD-PMO(4)) was custom-made and purchased from Gene Tools. A PMO with the sequence 5′-CCTCCGGTTCTGAAGGTGTTC-3′ (DMD-PMO(5)) [SEQ ID NO: 19] and a disulfide amide modification on the 3′ (3′-disulfidamide-DMD-PMO(5)) was custom-made and purchased from Gene Tools.

Anti-CD71 antibody (clone OKT9) targeting human CD71 (hCD71) and anti-CD71 antibody (clone R17 217.1.3) targeting murine (mCD71) were both purchased from BioXCell. Anti-CD63 antibody (clone NVG-2) targeting murine (mCD63) was purchased from Biolegend. IGF-1 ligand was purchased from PeproTech.

Tris(2-carboxyethyl)phosphine hydrochloride (TCEP, 98%, Sigma-Aldrich), 5,5-Dithiobis(2-nitrobenzoic acid) (DTNB, Ellman's reagent, 99%, Sigma-Aldrich), Zeba™ Spin Desalting Columns (2 mL, Thermo-Fisher), NuPAGE™ 4-12% Bis-Tris Protein Gels (Thermo-Fisher), NuPAGE™ MES SDS Running Buffer (Thermo-Fisher), Novex™ Sharp Pre-stained Protein Standard (Thermo-Fisher), PageBlue™ Protein Staining Solution (Thermo-Fischer), Pierce™ BCA Protein Assay Kit (Thermo-Fisher), N-Ethylmaleimide (NEM, 98%, Sigma-Aldrich), 1,4-Dithiothreitol (DTT, 98%, Sigma-Aldrich), Sephadex G25 (GE Healthcare), Sephadex G50 M (GE Healthcare), Superdex 200P (GE Healthcare), Isopropyl alcohol (IPA, 99.6%, VWR), Tris(hydroxymethyl)aminomethane (Tris, 99%, Sigma-Aldrich), Tris(hydroxymethyl)aminomethane hydrochloride (Tris.HCL, Sigma-Aldrich), L-Histidine (99%, Sigma-Aldrich), D-(+)-Trehalose dehydrate (99%, Sigma-Aldrich), Polyethylene glycol sorbitan monolaurate (TWEEN 20, Sigma-Aldrich), Dulbecco's Phosphate-Buffered Saline (DPBS, Thermo-Fisher), Guanidine hydrochloride (99%, Sigma-Aldrich), Ethylenediaminetetraacetic acid disodium salt dihydrate (EDTA-Na₂, 99%, Sigma-Aldrich), sterile filters 0.2 μm and 0.45 μm (Sartorius), Vivaspin T4 and T15 concentrator (Sartorius), Superdex 200PG (GE Healthcare), Tetra(ethylene glycol), Dimethyl sulfoxide (DMSO, 99%, Sigma-Aldrich), N-(2-Aminoethyl)maleimide trifluoroacetate salt (AEM, 98%, Sigma-Aldrich), L-Cysteine (98.5%, Sigma-Aldrich), deionized water (DI) was freshly taken from Ultrapure Lab Water Systems (MilliQ, Merck), Nickel-nitrilotriacetic acid agarose (Ni-NTA agarose, Protino), Glycine (99.5%, VWR), 5,5-Dithiobis(2-nitrobenzoic acid (Ellman's reagent, DTNB, 98%, Sigma-Aldrich), S-Acetylmercaptosuccinic anhydride Fluorescein (SAMSA reagent, Invitrogen) Sodium bicarbonate (99.7%, Sigma-Aldrich), Sodium carbonate (99.9%, Sigma-Aldrich), PD MiniTrap desalting columns with Sephadex G-25 resin (GE Healthcare), PD10 G25 desalting column (GE Healthcare), Zeba Spin Desalting Columns in 0.5, 2, 5, and 10 mL (Thermo-Fisher), Vivaspin Centrifugal Filters T4 10 kDa MWCO, T4 100 kDa MWCO, and T15 (Sartorius), Biosep s3000 aSEC column (Phenomenex), Vivacell Ultrafiltration Units 10 and 30 kDa MWCO (Sartorius), Nalgene Rapid-Flow filter (Thermo-Fisher), dichlormethan (Sigma-Aldrich), methanol (Sigma-Aldrich), diethyl ether (Sigma-Aldrich), acetonitrile (Sigma-Aldrich), Pyridine 2-thione (Sigma-Aldrich), Goat anti-Human IgG-HRP (Southern Biotech), Goat anti-Human Kappa—HRP (Southern Biotech), Tris concentrate (Thermo-Fisher), MOPS running buffer (20×, Thermo-Fisher), LDS sample buffer (4×, Thermo-Fisher), TBS Blocking Buffer (Thermo-Fisher), Tris (Tris(hydroxymethyl)aminomethane, Merck), Tris HCl (Sigma-Aldrich), Minisart RC15 0.2 μm filter (Sartorius), Minisart 0.45 μm filter (Sartorius), PD Minitrap G25 (Cytiva), TNBS (2,4,6-trinitrobenzene sulfonic acid, Sigma-Aldrich), Sodium Dodecyl Sulfate (SDS, Sigma-Aldrich), SMCC (succinimidyl 4-(N-maleimidomethyl)cyclohexane-1-carboxylate, Thermo-Fisher), THPP (Tris(hydroxypropyl)phosphine, Sigma-Aldrich), DBCO-NHS (CAS 1353016-71-3, BroadPharm), PEG4-SPDP (2-Pyridyldithiol-tetraoxatetradecane-N-hydroxysuccinimide, Thermo-Fisher), Novex™ TBE-Urea Gels, 15% (Thermo-Fisher), TBE buffer (ris-Borat-EDTA, Thermo-Fisher), GlyCLICK™ (10 mg) Azide Activation Kit (Genovis), and Immobilized GlycINATOR™ column (from GlyCLICK™ Azide Activation Kit) (Genovis) were used.

Materials Related to hCD71 Conjugates

| Description | Supplier |
|---|---|
| hCD71 human anti-CD71 antibody | BioXcell |
| SO1861-SC-Maleimide | Symeres |
| DMD-PMO(1)-5′-SS-amide (5′-DMD-PMO(1)) | Gene Tools |
| DMD-PMO(1)-SS-amide (3′-DMD-PMO(1)) | Gene Tools |
| DMD-PMO(2)-SS-amide (3′-DMD-PMO(2)) | Gene Tools |
| DMD-PMO(3)-SS-amide (3′-DMD-PMO(3)) | Gene Tools |
| DMD-PMO(4)-SS-amide (3′-DMD-PMO(4)) | Gene Tools |
| DMD-PMO(5)-SS-amide (3′-DMD-PMO(5)) | Gene Tools |
| DMD-ASO-thiol (5′-DMD-ASO) | Hanugen Therapeutics Pvt Ltd |
| Goat anti-Human IgG - HRP | Southern Biotech |
| Goat anti-Human Kappa - HRP | Southern Biotech |
| Conjugation buffer (TBS pH 7.5) | Fleet Bioprocessing Ltd |
| Dulbecco's PBS pH 7.5 | Fleet Bioprocessing Ltd |
| SEC analysis buffer (DPBS:IPA 85:15) | Fleet Bioprocessing Ltd |
| 6M Guanidine•HCl | Fleet Bioprocessing Ltd |
| 0.5M NaOH | Fleet Bioprocessing Ltd |
| PBS-T (Dulbecco's PBS pH 7.5 with Tween-20) | Fleet Bioprocessing Ltd |
| MOPS running buffer (20X) | Fleet Bioprocessing Ltd |
| LDS sample buffer (4X) | Thermo |
| NuPAGE Transfer Buffer (10X) | Fleet Bioprocessing Ltd |
| TBS Blocking Buffer | Thermo |
| TCEP | Thermo |
| THPP | Sigma |
| DTT | Sigma |
| PEG4-SPDP | Thermo |
| NEM | Sigma |
| Glycine | VWR |
| DMSO | Sigma |
| Ellman's reagent | Thermo |
| 0.2 μm Minisart Filter | Sartorius |
| 0.45 μm Minisart Filter | Sartorius |
| 0.2 μm Spin X Filter | Corning |
| Zeba 2 ml 7K MWCO | Thermo |
| Zeba 10 ml 40K MWCO | Thermo |
| PD10 G25 Sephadex | Cytiva |
| 2.6 × 30 cm Sephadex G25 | Fleet Bioprocessing Ltd |
| 2.6 × 40 cm Superdex 200 | Fleet Bioprocessing Ltd |
| 2.6 × 60 cm Superdex 200 | Fleet Bioprocessing Ltd |
| Vivaspin 20 (10K MWCO) | Sartorius |
| Vivaspin T15 (10K MWCO) | Sartorius |
| Vivacell 100 (10K MWCO) | Sartorius |
| Novex protein standards ladder | Life Technologies |
| 4-12% Bis-Tris gel | Thermo |
| Nitrocellulose membrane | Thermo |
| PageBlue protein stain | Thermo |
| Ultra TMB Blotting Solution | Thermo |
| BCA assay kit | Thermo |
| 2 mg/ml BGG standard | Thermo |
| Biosep s3000 aSEC column | Phenomenex |

### Abbreviations

Ab Antibody
Ac Acetyl
AON Antisense oligonucleotide
ASO Antisense oligonucleotide
BCA Bicinchoninic acid
BGG Bovine gamma globulin
aSEC Analytical size exclusion chromatography
DAR Drug-antibody ratio
DBCO Dibenzocyclooctyne
DBCO-NHS Dibenzocyclooctyne-N-hydroxysuccinimide ester
DCM Dichloromethane
DIPEA N,N-diisopropylethylamine
DMEM Dulbecco's modified Eagles medium
DMF N,N-dimethylformamide
DMSO Dimethylsulfoxide
DPBS Dulbecco's phosphate-buffered saline
DTME Dithiobismaleimidoethane
DTNB 5,5′-dithiobis-(2-nitrobenzoic acid)
DTT Dithiothreitol
EDTA Ethylenediaminetetraacetic acid
EDCI·HCl 1-(3-Dimethylaminopropyl)-3-ethylcarbodiimide hydrochloride
EMCH·TFA N-(ε-maleimidocaproic acid) hydrazide, trifluoroacetic acid salt
Equiv. Equivalent
EtBr Ethidium bromide
FBS Fetal bovine serum
GalT Galactose-1-phosphate uridylyl transferase
HATU 1-[Bis(dimethylamino)methylene]-1H-1,2,3-triazolo[4,5-b]pyridinium 3-oxide hexafluorophosphate
HRP Horse-radish peroxidase
IPA Isopropyl alcohol
LC-MS Liquid chromatography-mass spectrometry
LDS Lithium dodecyl sulfate
LRMS Low resolution mass spectrometry
NEM N-ethylmaleimide
NHS N-hydroxy succinimide ester
mAb Monoclonal antibody
min Minutes
MOPS 3-(Morpholin-4-yl)propane-1-sulfonic acid
MPLC Medium pressure liquid chromatography
MWCO Molecular weight cut-off
NMM 4-Methylmorpholine
PBS Phosphate-buffered saline
PBS-T Phosphate-buffered saline with Tween-20
PEG4-SPDP 2-Pyridyldithiol-tetraoxatetradecane-N-hydroxysuccinimide
PMO Phosphorodiamidate Morpholino Oligomer
PDT Pyridine 2-thione
rpm Revolutions per minute
RT Room temperature
r.t. Retention time
SDS Sodium dodecyl sulfate
SEC Size exclusion chromatography
SMCC Succinimidyl 4-(N-maleimidomethyl)cyclohexane-1-carboxylate
TBEU (Tris-(hydroxymethyl)-aminomethane)-Borate-EDTA-rea
TBS Tris buffered saline
TCEP Tris(2-carboxyethyl)phosphine hydrochloride
TCO4—NHS Trans-Cyclooctene-N-hydroxy succinimide
Temp Temperature
TFA Trifluoroacetic acid
THPP Tris(hydroxypropyl)phosphine
TMB 3,3′,5,5′-tetramethylbenzidine
TNBS 2,4,6-trinitrobenzene sulfonic acid
Tris Tris(hydroxymethyl)aminomethane
UDP-GalNAz Uridine diphosphate-N-azidoacetylgalactosamine

### Methods for Examples 1-4

### SO1861-Maleimides, SO1861-NHS Synthesis

SO1861 was from Saponaria officinalis L (Analyticon Discovery GmbH, Germany), and was coupled to respective handles by Symeres (NL) according to methods known in the art.

Conjugation of 5′-disulfidamide-DMD-PMO, 5′-thiol-DMD-ASO, and 3′-disulfidamide-M23D to Antibodies

Custom production of mCD71-M23D, mCD71-M23D-SO1861, and hCD71-DMD-ASO, hCD71-DMD-PMO, hCD71-DMD-ASO-SO1861, and hCD71-DMD-PMO-SO1861 was performed by Fleet Bioprocessing (UK).

### Analytical and Preparative Methods

### LC-MS Method 1

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: neg or neg/pos within in a range of 1500-2400 or 2000-3000; ELSD: gas pressure 40 psi, drift tube temp: 50° C.; column: Acquity C18, 50×2.1 mm, 1.7 μm Temp: 60° C., Flow: 0.6 mL/min, lin. Gradient depending on the polarity of the product:
^{A}t₀=2% A, t_{5.0min}=50% A, t_{6.0min}=98% A
^{B}t₀=2% A, t_{5.0min}=98% A, t_{6.0min}=98% A
Posttime: 1.0 min, Eluent A: acetonitrile, Eluent B: 10 mM ammonium bicarbonate in water (pH=9.5).

### LC-MS Method 2

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: pos/neg 100-800 or neg 2000-3000; ELSD: gas pressure 40 psi, drift tube temp: 50° C.; column: Waters XSelect™ CSH C18, 50×2.1 mm, 2.5 μm, Temp: 25° C., Flow: 0.5 mL/min, Gradient: t₀ₘᵢₙ=5% A, t_{2.0min}=98% A, t_{2.7min}=98% A, Posttime: 0.3 min, Eluent A: acetonitrile, Eluent B: 10 mM ammonium bicarbonate in water (pH=9.5).

### LC-MS Method 3

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product pos/neg 105-800, 500-1200 or 1500-2500; ELSD: gas pressure 40 psi, drift tube temp: 50° C.; column: Waters XSelect™ CSH C18, 50×2.1 mm, 2.5 μm, Temp: 40° C., Flow: 0.5 mL/min, Gradient: t₀ₘᵢₙ=5% A, t_{2.0min}=98% A, t_{2.7min}=98% A, Posttime: 0.3 min, Eluent A: 0.1% formic acid in acetonitrile, Eluent B: 0.1% formic acid in water.

### LC-MS Method 4

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: pos/neg 100-800 or neg 2000-3000; ELSD: gas pressure 40 psi, drift tube temp: 50° C. column: Waters Acquity Shield RP18, 50×2.1 mm, 1.7 μm, Temp: 25° C., Flow: 0.5 mL/min, Gradient: t₀ₘᵢₙ=5% A, t_{2.0min}=98% A, t_{2.7min}=98% A, Posttime: 0.3 min, Eluent A: acetonitrile, Eluent B: 10 mM ammonium bicarbonate in water (pH=9.5).

### Preparative MP-LC Method 1

Instrument type: Reveleris™ prep MPLC; column: Waters XSelect™ CSH C18 (145×25 mm, 10 μm); Flow: 40 mL/min; Column temp: room temperature; Eluent A: 10 mM ammoniumbicarbonate in water pH=9.0); Eluent B: 99% acetonitrile+1% 10 mM ammoniumbicarbonate in water; Gradient:
^{A}t₀ₘᵢₙ=5% B, t₁ₘᵢₙ=5% B, t₂ₘᵢₙ=10% B, t₁₇ₘᵢₙ=50% B, t₁₈ₘᵢₙ=100% B, t₂₃ₘᵢₙ=100% B
^{A}t₀ₘᵢₙ=5% B, t₁ₘᵢₙ=5% B, t₂ₘᵢₙ=20% B, t₁₇ₘᵢₙ=60% B, t₁₈ₘᵢₙ=100% B, t₂₃ₘᵢₙ=100% B; Detection UV: 210, 235, 254 nm and ELSD.

### Preparative MP-LC Method 2

Instrument type: Reveleris™ prep MPLC; Column: Phenomenex LUNA C18(3) (150×25 mm, 10 μm); Flow: 40 mL/min; Column temp: room temperature; Eluent A: 0.1% (v/v) Formic acid in water, Eluent B: 0.1% (v/v) Formic acid in acetonitrile; Gradient:
^{A}t₀ₘᵢₙ=5% B, t₁ₘᵢₙ=5% B, t₂ₘᵢₙ=20% B, t₁₇ₘᵢₙ=60% B, t₁₈ₘᵢₙ=100% B, t₂₃ₘᵢₙ=100% B
^{B}t₀ₘᵢₙ=2% B, t₁ₘᵢₙ=2% B, t₂ₘᵢₙ=2% B, t₁₇ₘᵢₙ=30% B, t₁₈ₘᵢₙ=100% B, t₂₃ₘᵢₙ=100% B
^{C}t₀ₘᵢₙ=5% B, t₁ₘᵢₙ=5% B, t₂ₘᵢₙ=10% B, t₁₇ₘᵢₙ=50% B, t₁₈ₘᵢₙ=100% B, t₂₃ₘᵢₙ=100% B
^{D}t₀min=5% B, t₁ₘᵢₙ=5% B, t₂ₘᵢₙ=5% B, t₁₇ₘᵢₙ=40% B, t₁₈ₘᵢₙ=100% B, t₂₃ₘᵢₙ=100% B; Detection UV: 210, 235, 254 nm and ELSD.

### Preparative LC-MS Method 3

MS instrument type: Agilent Technologies G6130B Quadrupole; HPLC instrument type: Agilent Technologies 1290 preparative LC; Column: Waters XSelect™ CSH (C18, 150×19 mm, 10 μm); Flow: 25 ml/min; Column temp: room temperature; Eluent A: 100% acetonitrile; Eluent B: 10 mM ammonium bicarbonate in water pH=9.0; Gradient:
^{A}t₀=20% A, t_{2.5min}=20% A, t₁₁ₘᵢₙ=60% A, t₁₃ₘᵢₙ=100% A, t₁₇ₘᵢₙ=100% A
^{B}t₀=5% A, t_{2.5min}=5% A, t₁₁ₘᵢₙ=40% A, t₁₃ₘᵢₙ=100% A, t₁₇ₘᵢₙ=100% A; Detection: DAD (210 nm);
Detection: MSD (ESI pos/neg) mass range: 100-800; Fraction collection based on DAD.

### Preparative LC-MS Method 4

MS instrument type: Agilent Technologies G6130B Quadrupole; HPLC instrument type: Agilent Technologies 1290 preparative LC; Column: Waters XBridge Protein (C4, 150×19 mm, 10 μm); Flow: 25 ml/min; Column temp: room temperature; Eluent A: 100% acetonitrile; Eluent B: 10 mM ammonium bicarbonate in water pH=9.0; Gradient:
^{A}t₀=2% A, t_{2.5min}=2% A, t₁₁ₘᵢₙ=30% A, t₁₃ₘᵢₙ=100% A, t₁₇ₘᵢₙ=100% A
^{B}t₀=10% A, t_{2.5min}=10% A, t₁₁ₘᵢₙ=50% A, t₁₃ₘᵢₙ=100% A, t₁₇ₘᵢₙ=100% A ^{C}t₀=5% A, t_{2.5min}=5% A, t₁₁ₘᵢₙ=40% A, t₁₃ₘᵢₙ=100% A, t₁₇ₘᵢₙ=100% A; Detection: DAD (210 nm);
Detection: MSD (ESI pos/neg) mass range: 100-800; Fraction collection based on DAD

### Flash Chromatography

Grace Reveleris X2™ C-815 Flash; Solvent delivery system: 3-piston pump with auto-priming, 4 independent channels with up to 4 solvents in a single run, auto-switches lines when solvent depletes; maximum pump flow rate 250 mL/min; maximum pressure 50 bar (725 psi); Detection: UV 200-400 nm, combination of up to 4 UV signals and scan of entire UV range, ELSD; Column sizes: 4-330 g on instrument, luer type, 750 g up to 3000 g with optional holder.

### UV-Vis Spectrophotometry

Concentrations were determined using either a Thermo Nanodrop 2000 spectrometer or Perkin Elmer Lambda 365 Spectrophotometer and the following mass Extinction Coefficient (EC) values:
Experimentally determined molar ε495=58,700 M-1 cm-1 and Rz280:495=0.428 were used for SAMSA-fluorescein.
M23D-SS-amide; mass EC265=259,210 M-1 cm-1
Ellman's reagent (TNB); molar EC412=14,150 M-1 cm-1
DMD-PMO; molar EC265=318,050 135,027 M-1 cm-1
DMD-ASO; molar EC265=310,000 252,512 M-1 cm-1
Pyridine 2-thione (PDT); molar E363=8,080 M-1 cm-1

### TNBS Assay

Glycine standards (0, 2.5, 5, 10, 15 and 20 μg/ml) were freshly prepared using DPBS pH 7.5. TNBS assay reagent was prepared by combining TNBS (40 μl) and DPBS pH 7.5 (9.96 ml). 10% w/v SDS prepared using DI water. For the assay, 60 μl of each sample (singlicate) and standard (triplicate) was plated out. To each well was added TNBS reagent (60 μl) and the plate shaker-incubated for 3 hours at 37° C. and 600 rpm. After, 50 μl of 10% SDS and 25 μl 1 M HCl was added and the plate was analysed at 340 nm. SO1861-hydrazone-NHS incorporation was determined by depletion of lysine concentration of conjugate with respect to unmodified protein.

### SEC

The conjugates were analysed by SEC using an Akta purifier 10 system and Biosep SEC-s3000 column eluting with DPBS:IPA (85:15). Conjugate purity was determined by integration of the conjugate peak with respect to impurities/aggregate forms.

### SDS-PAGE and Western Blotting

Native proteins and conjugates were analysed under heat denaturing non-reducing and reducing conditions by SDS-PAGE against a protein ladder using a 4-12% bis-tris gel and MOPS as running buffer (200V, ˜40 minutes). Samples were prepared to 0.5 mg/ml, comprising LDS sample buffer and MOPS running buffer as diluent. For reducing samples, DTT was added to a final concentration of 50 mM. Samples were heat treated for 2 minutes at 90-95° C. and 5 μg (10 μl) added to each well. Protein ladder (10 μl) was loaded without pre-treatment. Empty lines were filled with 1×LDS sample buffer (10 μl). After the gel was run, it was washed thrice with DI water (100 ml) with shaking (15 minutes, 200 rpm). Coomassie staining was performed by shaker-incubating the gel with PAGEBlue protein stain (30 ml) (60 minutes, 200 rpm). Excess staining solution was removed, rinsed twice with DI water (100 ml) and destained with DI water (100 ml) (60 minutes, 200 rpm). The resulting gel was imaged and processed using ImageJ (ImageJ (Rasband, W. S., ImageJ, U. S. National Institutes of Health, Bethesda, Maryland, USA) and MyCurveFit (point-to-point correlation of protein ladder).

### Western Blotting

From SDS-PAGE, the gel was transferred to nitrocellulose membrane using the X-Cell blot module with the following setup ((−)BP-BP-FP-Gel-NC-BP-BP-BP(+)) and conditions (30V, 60 minutes) using freshly prepared transfer buffer. BP—blotting pad; FP—Filter pad; NC—Nitrocellulose membrane. After that, the NC were washed thrice with PBS-T (100 ml) with shaking (5 minutes, 200 rpm), non-specific sites blocked with blocking buffer (50 ml) with shaking (30 minutes, 200 rpm) then active sites labelled with a combination of Goat anti-Human Kappa—HRP (1:2000) and Goat anti-Human IgG—HRP (1:2000) (50 ml) diluted in blocking buffer with shaking (30 minutes, 200 rpm). After that, the NC was washed once with PBS-T (100 ml) with shaking (5 minutes, 200 rpm) and complexed antibody detected with freshly prepared, freshly filtered CN/DAB substrate (25 ml). Colour development was observed visually, and after 2 minutes development was stopped by washing the NC with water, and the resulting blot photographed.

### TBEU-PAGE

Oligonucleotide conjugates and oligonucleotide standards were analysed under heat denaturing, non-reducing conditions by TBE-Urea PAGE against an oligo ladder using a 15% TBE-Urea gel and TBE as running buffer (180V, ˜60 minutes). Samples were prepared to 0.5 mg/ml, and standards were prepared to 50 to 5 μg/ml, respectively, all comprising TBE Urea sample buffer and purified H₂O as diluent. Samples and standards were heat treated for 3 minutes at 70° C. and 10 μl added to each well, equating to 5 μg of protein and conjugate samples, and 0.5/0.2/0.1/0.5 μg (DMD-ASO) or 0.2/0.1/0.05 μg (DMD-PMO) of oligonucleotide, per lane. Oligo ladder reconstituted to 0.1 μg/band/ml in TE pH 7.5 (2 μl) was loaded without pre-treatment. After the gel was run, it was stained with freshly prepared ethidium bromide solution (1 μg/ml) with shaking (40 minutes, 200 rpm). The resulting gel was visualised by UV epi-illumination (254 nm), imaged and processed using ImageJ (Rasband, W. S., ImageJ, U. S. National Institutes of Health, Bethesda, Maryland, USA).

mCD71-M23D

An aliquot of mCD71 (42.9 mg, 4.20 ml) was buffer exchanged into DPBS pH 7.5 and normalised to 2.5 mg/ml. To an aliquot of mCD71 (34.4 mg, 0.23 μmol, 2.53 mg/ml) was added an aliquot of freshly prepared SMCC solution (2.0 mg/ml, 3.53 mole equivalents, 0.81 μmol), the mixture vortexed briefly then incubated for 60 minutes at 20° C. with roller-mixing. After incubation, the reaction was quenched by the addition of an aliquot of a freshly prepared glycine solution (2.0 mg/ml, ˜20 mole equivalents, 4.05 μmol), the mixture vortexed briefly then incubated for >15 minutes at 20° C. with roller-mixing. The conjugate was purified by Superdex 200 column eluting with TBS pH 7.5 and analysed by UV-vis to give purified mCD71-SMCC (31.7 mg, yield: 96%, 0.942 mg/ml, SMCC to mCD71 ratio=2.1).

Separately, an aliquot of M23D-SS-amide (17.2 mg, 1.99 μmol, 10.0 mg/ml) reconstituted using TBS pH 7.5 was added an aliquot of freshly prepared THPP solution (50 mg/ml, 10 mole equivalents, 19.9 μmol, 82.8 μl), the mixture vortexed briefly then incubated for 60 minutes at 37° C. with roller-mixing. After incubation, the oligo was purified by PD10 Sephadex G25M column eluting with TBS pH 7.5, to afford M23D-SH (14.8 mg, yield: 86%, thiol to M23D ratio=0.98).

To an aliquot of mCD71-SMCC (31.7 mg, 0.21 μmol, 0.942 mg/ml) was added an aliquot of M23D-SH (4.122 mg/ml, 4.0 mole equivalents, 0.85 μmol, 1.771 ml), the mixture vortexed briefly then incubated at 20° C. with roller-mixing. After ca. 72 hours, the conjugate mixture was concentrated and purified by Superdex200PG column eluting with DPBS pH 7.5 to give purified mCD71-M23D conjugate. The aliquot was analysed by BCA colorimetric assay and assigned a new EC value for the conjugate, then concentrated and normalised to 2.5 mg/ml, filtered through 0.2 μm and then dispensed into an aliquot for characterisation and an aliquot for product testing. The result was a mCD71-M23D conjugate (total yield=25.7 mg, 73%, M23D to mCD71 ratio=1.2).

hCD71-DMD-ASO

An aliquot of hCD71 (60 mg, 4.20 ml) was buffer exchanged into DPBS pH 7.5 and normalised to 2.5 mg/ml. To an aliquot of hCD71 (58 mg, 0.38 μmol, 2.53 mg/ml) was added an aliquot of freshly prepared PEG4-SPDP solution (10 mg/ml, 10 mole equivalents, 3.8 μmol), the mixture vortexed briefly then incubated for 60 minutes at 20° C. with roller-mixing. After incubation, the reaction was quenched by the addition of an aliquot of a freshly prepared glycine solution (2.0 mg/ml, 50 mole equivalents, 19 μmol), the mixture vortexed briefly then incubated for >15 minutes at 20° C. with roller-mixing. The conjugate was purified by Zeba 40K spin desalting column eluting with TBS pH 7.5 and analysed by UV-vis to give purified hCD71-PEG4-SPDP (51.3 mg, yield: 88%, 0.95 mg/ml, PEG4-SPDP to hCD71 ratio=4.5).

Separately, an aliquot of DMD-ASO-SH (14.4 mg, 2 μmol, 10.0 mg/ml) reconstituted using TBS pH 7.5 was added an aliquot of freshly prepared THPP solution (50 mg/ml, 10 mole equivalents, 20 μmol, 82.8 μl), the mixture vortexed briefly then incubated for 60 minutes at 37° C. with roller-mixing. After incubation, the oligo was purified by PD10 Sephadex G25M column eluting with TBS pH 7.5, to afford reduced DMD-ASO-SH (13.2 mg, yield: 92%, thiol to DMD-ASO ratio=0.91).

To an aliquot of hCD71-PEG4-SPDP (25 mg, 0.16 μmol, 0.95 mg/ml) was added an aliquot of DMD-ASO-SH (4 mg/ml, 4.0 mole equivalents, 0.65 μmol, 1.17 ml), the mixture vortexed briefly then incubated at 20° C. with roller-mixing. Reaction progression was measured by PDT displacement. After 16 hours, the conjugate mixture was concentrated and purified by Superdex 200PG column eluting with DPBS pH 7.5 to give purified hCD71-DMD-ASO conjugate. The aliquot was analysed by BCA colorimetric assay and assigned a new EC value for the conjugate, then concentrated and normalised to 2.5 mg/ml, filtered through 0.2 μm and then dispensed into an aliquot for characterisation and an aliquot for product testing. The result was a hCD71-DMD-ASO conjugate (total yield=23.1 mg, 82%, DMD-ASO to hCD71 ratio=3.5). In a second synthesis, a hCD71-DMD-ASO conjugate was synthesized with the same methods as described and a DMD-ASO to hCD71 ratio=2.1).

hCD71-DMD-PMO

An aliquot of hCD71 (60 mg, 4.20 ml) was buffer exchanged into DPBS pH 7.5 and normalised to 2.5 mg/ml. To an aliquot of hCD71 (58 mg, 0.38 μmol, 2.53 mg/ml) was added an aliquot of freshly prepared PEG4-SPDP solution (10 mg/ml, 10 mole equivalents, 3.8 μmol), the mixture vortexed briefly then incubated for 60 minutes at 20° C. with roller-mixing. After incubation, the reaction was quenched by the addition of an aliquot of a freshly prepared glycine solution (2.0 mg/ml, 50 mole equivalents, 19 μmol), the mixture vortexed briefly then incubated for >15 minutes at 20° C. with roller-mixing. The conjugate was purified by Zeba 40K spin desalting column eluting with TBS pH 7.5 and analysed by UV-vis to give purified hCD71-PEG4-SPDP (51.3 mg, yield: 88%, 0.95 mg/ml, PEG4-SPDP to hCD71 ratio=4.1).

Separately, an aliquot of DMD-PMO-SS-amide (20.2 mg, 2 μmol, 10.0 mg/ml) reconstituted using TBS pH 7.5 was added an aliquot of freshly prepared THPP solution (50 mg/ml, 10 mole equivalents, 20 μmol, 82.8 μl), the mixture vortexed briefly then incubated for 60 minutes at 37° C. with roller-mixing. After incubation, the oligo was purified by PD10 Sephadex G25M column eluting with TBS pH 7.5, to afford DMD-PMO-SH (16.4 mg, yield: 81%, thiol to DMD-PMO ratio=0.97).

To an aliquot of hCD71-PEG4-SPDP (25 mg, 0.16 μmol, 0.95 mg/ml) was added an aliquot of DMD-PMO-SH (4.1 mg/ml, 4.0 mole equivalents, 0.65 μmol, 1.59 ml), the mixture vortexed briefly then incubated at 20° C. with roller-mixing. Reaction progression was measured by PDT displacement. After ca 16 hours, the conjugate mixture was concentrated and purified by Superdex 200PG column eluting with DPBS pH 7.5 to give purified hCD71-DMD-PMO conjugate. The aliquot was analysed by BCA colorimetric assay and assigned a new EC value for the conjugate, then concentrated and normalised to 2.5 mg/ml, filtered through 0.2 μm and then dispensed into an aliquot for characterisation and an aliquot for product testing. The result was a hCD71-DMD-PMO conjugate (total yield=28.2 mg, 92%, DMD-PMO to hCD71 ratio=3.9). In a second synthesis, a hCD71-DMD-PMO conjugate was synthesized with the same method, and a DMD-PMO to hCD71 ratio=3.2).

### Cell Culture (Human)

Immortalized human myoblasts from non-DMD donors (KM155) and myoblasts from a DMD-affected donor (DM8036) were cultured in Skeletal Muscle Cell Growth Medium (PromoCell, Germany) with supplementary pack according to manufacturer's instructions, further supplemented with 15% fetal bovine serum (Gibco, United Kingdom), and 0.5% gentamicin (Sigma-Aldrich, USA). For differentiation, cells were seeded on a 0.5% gelatine-coated surface, and at ˜70-80% confluence, the proliferation medium was replaced by DMEM (Gibco) supplemented with 2% FBS (Gibco), 2% GlutaMAX, and 1% glucose (Sigma-Aldrich) and 0.5% gentamycin. Treatments were started after at least 3 days up to a maximum of 5 days of differentiation (based on the presence of differentiated myotubes).

### Cell Culture and In Vitro Experiments (Murine)

Murine myoblast cell line C2C12 was maintained in 10% FBS DMEM medium+Pen/Strep and plated at 240,000 cells per well (cpw) in 24-well plates or 40,000 cpw in 96-well plates (wp) in maintenance medium (10% FBS in DMEM medium+Pen/Strep) and incubated at 37° C. with 5% CO₂. Twenty-four hours after seeding, cells were switched to differentiation media (2% horse serum in DMEM) and incubated for 3 days before refreshing the medium. After another 24 hours, medium was refreshed again and compounds were added and incubated for 48 hours. Differentiation medium was then refreshed (without compounds), and cells were incubated for another 24 hours. At 72 hours total post treatment, cells in the 24-wp were harvested for exon skip analysis and the cell viability was assessed on the 96-wp.

### Exon Skip Analysis and Quantification (Human)

RNA was isolated with the TRIsure Isolation Reagent (Bioline) and chloroform extraction; isopropanol precipitation of RNA from the aqueous phase was performed as known to someone skilled in the art. For cDNA synthesis, 1000 ng of total RNA was used and diluted in an appropriate amount of RNase-free water to yield 8 μl RNA dilution. The priming premixed contained 1 μl dNTP mix (10 mM each) and 1 μl specific reverse primer (for KM155, h53R 5′-CTCCGGTTCTGAAGGTGTTC-3′ [SEQ ID NO: 5]; for DM8036, h55R 5′-ATCCTGTAGGACATTGGCAGTT-3′ [SEQ ID NO: 6]). This mixture was heated for 5 min at 70° C., then chilled on ice for at least 1 min. A reaction mixture was prepared containing 0.5 μl rRNasin (Promega), 4.0 μl RT buffer, 1.0 μl Tetro RT (Bioline), and 4.5 μl RNase-free water, and was added to the chilled mixture to yield a total volume of 20 μl per reaction. The RT-PCR was run for 60 min at 42° C., then 5 min at 85° C., and chilled on ice. For skip analysis, a nested PCR approach was followed. To this end, in the first PCR, 3 μl cDNA were added to a mix of 2.5 μl 10×SuperTaq PCR buffer, 0.5 μl dNTP mix (10 mM each), 0.125 μl Taq DNA polymerase TAQ-RO (5 U/μl; Roche) 16.875 μl RNase-free water and 1 μl (10 pmol/μl) of each primer flanking the targeted exons were used: for KM155, h48F 5′-AAAAGACCTTGGGCAGCTTG-3′ [SEQ ID NO: 7] and h53R 5′-CTCCGGTTCTGAAGGTGTTC-3′ [SEQ ID NO: 5]; for DM8036, h47F2 5′-TGAAACTGGAGGACCCGTG-3′ [SEQ ID NO: 8] and h54R 5′-CCAAGAGGCATTGATATTCTC-3′ [SEQ ID NO: 9]. These samples were subjected to a PCR run of 5 min at 94° C., then 25 cycles with 40 sec at 94° C., 40 sec at 60° C., 180 sec at 72° C., after which for 7 min at 72° C. For the second PCR, 1.5 μl PCR1 sample were added to a mix of 5 μl 10×SuperTaq PCR buffer, 1 μl dNTP mix (10 mM each), 0.25 μl Taq DNA polymerase TAQ-RO (5 U/μl; Roche), 38.25 μl RNase-free water and 2 μl (10 pmol/μl) of each primer flanking the targeted exons were used: for KM155, h49F 5′-CCAGCCACTCAGCCAGTG-3′ [SEQ ID NO: 10] and h52R2 5′-TTCTTCCAACTGGGGACGC-3′ [SEQ ID NO: 11]; for DM8036, h47F 5′-CCCATAAGCCCAGAAGAGC-3′ [SEQ ID NO: 12] and h53R 5′-CTCCGGTTCTGAAGGTGTTC-3′ [SEQ ID NO: 13]. These samples were subjected to a PCR run of 5 min at 94° C., then 32 cycles with 40 sec at 94° C., 40 sec at 60° C., 60 sec at 72° C., after which for 7 min at 72° C. Exon skipping levels were quantified with the Femto Pulse System using the Ultra Sensitivity NGS Kit (Agilent), according to the manufacturer's instructions. Alternatively, the specific PCR fragments were analyzed using Bioanalyzer 2100 with DNA1000 chip (lab-on-a-chip; Agilent), or separated on 2% agarose gels run at 120 V. Gels were imaged and band intensities were quantified using ImageJ. The expected non-skipped product has a size of 408 bp (KM155) and 475 bp (DM8036), and the skip product of 175 bp (KM155) or 242 bp (DM8036), respectively.

### Exon Skip Analysis and Quantification (Murine Vitro and Vivo)

For analysis of skip from murine cell lines, cells were harvested and RNA was isolated using 0.5 ml TRIzol™ Reagent (Thermo Scientific) per sample, according to the manufacturer's instruction. For analysis of tissue, 30-50 mg frozen tissue was first cut into smaller pieces and mRNA was isolated using TRIzol™ Reagent (Thermo Scientific) and a TissueLyser LT (Qiagen) according to the manufacturer's instruction. For cDNA synthesis per sample, to 0.5 μg RNA in 5.0 μl, 10.0 μl ddH₂O, 4.0 μl 5×iScript™ Reaction Mix and 1.0 μl iScript™ Reverse Transcriptase (BioRad) were added to yield a total volume of 20.0 μl per reaction. The RT-PCR was run for 5 min at 25° C., 60 min at 46° C., and 2 min at 95° C. For skip analysis, SapphireAmp™ Fast PCR Master Mix (TakaraBio) was used according to the manufacturer's instructions. To this end, 9.7 μl RNAse free water, 12.5 μl of 2× Master Mix, and 0.4 μl of 10 μM FW primer 5′-ACCCAGTCTACCACCCTATC-3′ (SEQ ID NO: 14) and 0.4 μl of 10 μM RV primer 5′-CTCTTTATCTTCTGCCCACCTT-3′ (SEQ ID NO: 15) were added to a PCR tube, mixed, after which 2 μl cDNA (50 ng) was added, to yield a total volume of 25 μl. These samples were subjected to a PCR run of 1 min at 94° C., 35 cycles of 5 sec at 98° C., 5 sec at 55° C., 5 sec at 72° C., followed by 1 min at 72° C. Samples were mixed with 2 μl 6× loading buffer, then 16 μl were loaded onto an 2% agarose gel and ran for 60-90 min at 80 V. Gels were imaged and band intensities were quantified with a ChemiDoc™ XRS+System and Image Lab™ Software (BioRad). The expected non-skipped product has a size of 788 bp, and the skip product of 575 bp, respectively.

### Cell Viability (Murine Vitro)

After treatment the cell viability was determined with a CellTiter-Glo™ 2.0 assay, performed according to the manufacturer's instruction (Promega). The luminescence signal was measured on a SpectraMax ID5 plate reader (Molecular Devices). For quantification, the background signal of ‘medium only’ wells was subtracted from all other wells, before the cell viability percentage of treated/untreated cells was calculated, by dividing the background corrected signal of treated wells over the background corrected signal of the untreated wells (×100).

### In Vivo Studies

Per group, 9 male CD-1 mice, aged 6-7 weeks at dosing, were given a single injection intravenously (iv) of the compound or vehicle listed in Table A2. During the treatment period, animals were regularly weighed (on the day before dosing, and twice weekly post dosing) and any clinical observations were recorded. At day 4, day 14, and day 28, respectively, 3 mice per group were sacrificed and terminal bleeds and samples from different tissues and organs were harvested for analysis. Serum was prepared and ALT (AU480, Beckman Coulter) and creatinine levels (colorimetric method, Beckman Coulter) were analyzed. Tissues were preserved in RNALater and snap frozen until analysis. In heart, diaphragm and gastrocnemius samples, dystrophin skip levels were determined.

TABLE A2
Dosing groups in vivo efficacy and tolerability

| | | | | Relative PMO |
|---|---|---|---|---|
| | | | Total | amount in total |
| | | # | compound | dose |
| Group | Compound Name | animals | [mg/kg] | [mg/kg] |
| 1 | Vehicle (PBS) | 9 | 0.0 | N/A |
| 2 | mCD71-M23D | 9 | 44.0 | 2.80 |
| | (DAR1.2) | | | |

### Methods for Example 5

### SO1861-SC-Maleimide

SO1861 was from Saponaria officinalis L (Extrasynthese, France) and was coupled to respective handle by Symeres (NL), according to methods known in the art.

### Synthesis of DBCO-(M23D)₂

DBCO-(M23D)₂ synthesis was performed by Symeres (NL).

### Conjugation of DBCO-(M23D)₂ to Antibodies

Custom conjugate productions of mCD71-M23D and mCD63-M23D were performed by Abzena (UK).

### Analytical and Preparative Methods

### Analytical Methods

### SEC Method 1

Apparatus for reaction analysis: Analytical SEC Instrument DIONEX Ultimate 3000 UPLC (DIONEX 6); Column: Waters Protein BEH SEC Column, 200 Å, 1.7 μm, 4.6 mm×150 mm; Mobile Phase: Buffer A (0.2 M Potassium Phosphate buffer, pH 6.8, 0.2M KCl, 15% isopropanol in ultra-pure water); Method: Isocratic buffer A for 10 min; Flow Rate: 0.35 ml/min; Run Time: 10 min; Detection UV: 214 nm, 248 nm, 260 nm and 280 nm; Column Oven: 30° C.; Auto Sampler: ambient; Injection Volume: 10 μL; Sample preparation: final sample was analyzed by diluting sample to 1.0 mg/ml with DPBS.

### LC-MS Method 1

Mass Spectrometry (LC-MS) Instrument: XEVO-G2XS TOF; Column: Agilent Poroshell 300SB-C3 guard, 5 μm RP column; Mobile Phases: Buffer A (Water, 0.1% formic acid) Buffer B (MeCN, 0.1% formic acid); Inlet Method: 0-2.0 min, 10% B 2.0-7.0 min, 10-80% B 7.0-8.0 min, 100% B 8.0-8.01 min, 10% B 8.01-10.0 min, 10% B; MS Method: Capillary voltage: 3.0 kV, Cone voltage: 120 V, Cone temperature: 140° C., Desolvation temperature: 450° C.: Flow Rate: 0.4 ml/min: Run Time: 10 min: Detection: TIC; Column Oven: 60° C.; Auto Sampler: Ambient; Injection Volume: 10 μL; Sample preparation: a. Final samples, intermediates, and reaction mixtures were analyzed by diluting sample to 0.05 mg/ml in DPBS.

### LC-MS Method 2

Instrument: Agilent 1260 Infinity II, 1260 G7112B Bin. Pump, 1260 G7167A Multisampler, 1290 MCT G7116B Column Comp. 1260 G7115A DAD (210, 220 and 210-320 nm), PDA (210-320 nm), G6130B MSD (ESI pos/neg) mass range 90-1500, Column: XSelect CSH C18 (30×2.1 mm 3.5 μm) Flow: 1 ml/min, Column temp: 40° C., Eluent A: 0.1% formic acid in Water, Eluent B: 0.1% formic acid in acetonitrile, Gradient: t₀ₘᵢₙ=5% B, t_{1.6min}=98% B, t₃ₘᵢₙ=98% B, Postrun: 1.3 min.

### LC-MS Method 3

Instrument: Agilent 1260 Infinity II, 1260 G7112B Bin. Pump, 1260 G7167A Multisampler, 1260 MCT G7116A Column Comp. 1260 G7115A DAD (210, 220 and 210-320 nm), PDA (210-320 nm), G6130B MSD (ESI pos/neg) mass range 90-1500, Column: XSelect CSH C18 (30×2.1 mm 3.5 μm), Flow: 1 ml/min, Column temp: 25° C., Eluent A: 10 mM ammoniumbicarbonate in water (pH 9.5), Eluent B: acetonitrile, Gradient: t₀ₘᵢₙ=5% B, t_{1.6min}=98% B, t₃ₘᵢₙ=98% B, Postrun: 1.2 min.

### LC-MS Method 4

Instrument: Agilent 1260 Infinity II, 1260 G7112B Bin. Pump, 1260 G7167A Multisampler, 1290 MCT G7116B Column Comp. 1260 G7115A DAD (210-320 nm, 210 and 220 nm), PDA (210-320 nm), G6130B MSD (ESI pos/neg) mass range 90-1500, Column: Waters C4 BEH (50×2.1 mm 3.5 μm) Flow: 1 ml/min; Column Temp: 40° C., Eluent A: 0.1% formic acid in water, Eluent B: 0.1% formic acid in acetonitrile, Gradient:
^{A}t₀ₘᵢₙ=5% B, t_{2.5min}=98% B, t₄ₘᵢₙ=98% B
^{B}t₀ₘᵢₙ=5% B, t_{0.05min}=5% B, t₅ₘᵢₙ=98% B, t₆ₘᵢₙ=98% B
Postrun: 1.5 min.

### Preparative Methods

### Preparative MP-LC Method 1

Instrument type: Buchi Reveleris™ prep MPLC; column: Waters XSelect™ CSH C18 (145×25 mm, 10 μm); Flow: 40 ml/min; Column temp: room temperature; Eluent A: 10 mM ammoniumbicarbonate in water pH=9.0); Eluent B: 99% acetonitrile+1% 10 mM ammoniumbicarbonate in water; Gradient: t₀ₘᵢₙ=50% B, t₄ₘᵢₙ=50% B, t₁₆ₘᵢₙ=100% B, t₂₁ₘᵢₙ=100% B; Detection UV: 220, 254, 270 nm; Fraction collection based on UV.

### Preparative MP-LC Method 2

Instrument type: Buchi Reveleris™ prep MPLC; Column: Phenomenex LUNA C18(3) (150×25 mm, 10 μm); Flow: 40 ml/min; Column temp: room temperature; Eluent A: 0.1% (v/v) formic acid in water, Eluent B: 0.1% (v/v) formic acid in acetonitrile; Gradient: t₀ₘᵢₙ=5% B, t₁ₘᵢₙ=5% B, t₂ₘᵢₙ=20% B, t₁₇ₘᵢₙ=60% B, t₁₈ₘᵢₙ=100% B, t₂₃ₘᵢₙ=100% B; Detection UV: 220, 240, 280 nm; Fraction collection based on UV.

### Preparative LC-MS Method

MS instrument type: Agilent Technologies G6120AA Quadrupole; HPLC instrument type: Agilent Technologies 1200 preparative LC; Column: Waters XBridge Protein (C4, 150×19 mm, 10μ); Flow: 25 ml/min; Column temp: room temperature; Eluent A: 0.1% formic acid in water; Eluent B: 100% acetonitrile; Gradient: to =10% A, t_{2.5min}=10% A, t₁₁ₘᵢₙ=50% A, t₁₃ₘᵢₙ=100% A, t₁₇ₘᵢₙ=100% A; Detection: DAD (220-320 nm); Detection: MSD (ESI pos/neg) mass range: 100-1000; Fraction collection based on DAD.

### Flash Chromatography

Grace Reveleris X2™ C-815 Flash; Solvent delivery system: 3-piston pump with auto-priming, 4 independent channels with up to 4 solvents in a single run, auto-switches lines when solvent depletes; maximum pump flow rate 250 ml/min; maximum pressure 50 bar (725 psi); Detection: UV 200-400 nm, combination of up to 4 UV signals and scan of entire UV range, ELSD; Column sizes: 4-330 g on instrument, luer type, 750 g up to 3000 g with optional holder.

### DBCO-(M23D)₂ Synthesis

### Intermediate 1 (tert-butyl N-[2-(4-{2-azatricyclo[10.4.0.0^{4,9}]hexadeca-1(12),4(9),5,7,13,15-hexaen-10-yn-2-yl}-N-(2-{[(tert-butoxy)carbonyl]amino}ethyl)-4-oxobutanamido)ethyl]carbamate)

To a solution of DBCO-acid (50.0 mg, 0.164 mmol) in DMF (1.00 ml) was added DIPEA (34.0 μL, 0.195 mmol) and HATU (62.3 mg, 0.164 mmol) and the mixture was stirred for 15 min. Next, di-tert-butyl (azanediylbis(ethane-2,1-diyl))dicarbamate (59.6 mg, 0.197 mmol) was added and the reaction mixture was stirred at room temperature. After 30 min, the reaction mixture was added to water (10.0 ml). The resulting dense suspension was centrifuged (5000 RPM, 3 min) to yield a clear solution with solids on the top. The solution was removed with a pipette and the solids were dissolved in acetonitrile (10.0 ml). The resulting solution was concentrated in vacuo. The residue was purified by flash chromatography (DCM—methanol/DCM (1/9, v/v) gradient 100:0 rising to 0:50) to give the title product (90.0 mg, 93%) as a colorless solidifying oil. Purity based on LC-MS 100%.

LRMS (m/z): 591 [M+H]¹⁺

LC-MS r.t. (min): 2.121

### Intermediate 2 (N,N-bis(2-azaniumylethyl)-4-{2-azatricyclo[10.4.0.0^{4,9}]hexadeca-1(12),4(9),5,7,13,-15-hexaen-10-yn-2-yl}-4-oxobutanamide ditrifluoroacetate)

Tert-butyl N-[2-(4-{2-azatricyclo[10.4.0.0^{4,9}]hexadeca-1(12),4(9),5,7,13,15-hexaen-10-yn-2-yl}-N-(2-{[(tert-butoxy)carbonyl]amino}ethyl)-4-oxobutanamido)ethyl]carbamate (90.0 mg, 0.152 mol) was dissolved in DCM (2.00 ml) and cooled to 0° C. Next, TFA (2.00 ml, 26.0 mmol) was added and the reaction mixture was stirred at 0° C. for 40 min. The reaction mixture was allowed to reach room temperature over the course of 10 min. As next, the reaction mixture was cooled to 0° C. and diluted with toluene (5 ml). The resulting solution was concentrated in vacuo and co-evaporated with DCM (2×5 ml) to give the crude title product as a slightly pink oil, which was used directly in the next step. Purity based on LC-MS 88%.

LRMS (m/z): 196 [M+2]²⁺, 391 [M+1]¹⁺

LC-MS r.t. (min): 1.17 (LC-MS method 2)

### Intermediate 3 (1S,4E)-cyclooct-4-en-1-yl N-[2-(4-{2-azatricyclo[10.4.0.0^{4,9}]hexadeca-1(12),4(9),5,-7,13,15-hexaen-10-yn-2-yl}-N-[2-({[(1S,4E)-cyclooct-4-en-1-yloxy]carbonyl}amino)ethyl]-4-oxobutan-amido)ethyl]carbamate

To a solution of crude N,N-bis(2-azaniumylethyl)-4-{2-azatricyclo[10.4.0.0^{4,9}]hexadeca-1(12),4(9),-5,7,13,15-hexaen-10-yn-2-yl}-4-oxobutanamide ditrifluoroacetate (0.152 mmol) in DMF (1.00 ml) and DIPEA (200 μL, 1.15 mmol) was added TCO4—NHS carbonate (102 mg, 0.380 mmol) and the mixture was stirred at room temperature. After 30 min the reaction mixture was submitted to preparative MP-LC (Preparative MP-LC method 1). Fractions corresponding to the product were immediately pooled together, frozen and lyophilized overnight to give the title compound (90.0 mg, 85%) as a slightly brown oil. Purity based on LC-MS 99%.

LRMS (m/z): 696 [M+1]¹⁺

LC-MS r.t. (min): 2.35 (LC-MS method 3)

### Intermediate 4 2-[4-(6-methyl-1,2,4,5-tetrazin-3-yl)phenyl]-N-[2-(pyridin-2-yldisulfanyl)ethyl]acetamide

To a solution of methyltetrazine-NHS ester (50.0 mg, 0.153 mmol) in DMF (800 μL) was added 2-(2-pyridinyldisulfanyl)ethanamine hydrochloride (40.8 mg, 0.183 mmol) and DIPEA (53.0 μL, 0.306 mmol). The resulting mixture was stirred at room temperature. After two hours the reaction mixture was submitted to preparative MP-LC (Preparative MP-LC method 2). Fractions corresponding to the product were immediately pooled together, frozen and lyophilized overnight to give the title compound (53.6 mg, 88%) as a pink solid. Purity based on LC-MS 100%.

LRMS (m/z): 399 [M+1]¹⁺

LC-MS r.t. (min): 1.87 (LC-MS method 3)

### Intermediate 5: M23D-mTz

To a solution of M23D-DSA (294 mg, 34.0 μmol) in water (15.0 ml) was added DTT (100 mg, 648 μmol). The reaction mixture was stirred at room temperature. After two hours, the reaction mixture was divided in six equal fractions and poured in acetonitrile (6×45 ml). The resulting suspensions were shaken and left standing for 30 min. Next, the suspensions were centrifuged (7830 RPM, 20 min). The solutions were decanted and the residues were treated with acetonitrile (each vial 20 ml). The resulting suspensions were centrifuged (7830 RPM, 3 min). The residues were dissolved in water (total 10.0 ml) and the solutions were combined. Next, a solution of 2-[4-(6-methyl-1,2,4,5-tetrazin-3-yl)phenyl]-N-[2-(pyridin-2-yldisulfanyl)ethyl]acetamide (54.2 mg, 136 μmol) in acetonitrile (4.00 ml) was added. The reaction mixture was stirred at room temperature. After two hours the reaction mixture was equally divided and poured in acetonitrile (6×45 ml). The resulting suspensions were shaken and centrifuged (7830 RPM, 3 min). The solutions were decanted and the residues were dissolved in water/acetonitrile (6×2 ml, 1/1, v/v). The resulting solutions were poured in acetonitrile (6×20 ml). Next, the suspensions were centrifuged (7830 RPM, 3 min). The solutions were decanted and the residues were dissolved in water/acetonitrile (total 15 ml, 1/1, v/v) and lyophilized overnight to give the title compound (340 mg, quant.) as a pink solid. Purity based on LC-MS 99%.

LRMS (m/z): 1468 [M+6]⁶⁺, 1259 [M+7]⁷⁺, 1102 [M+8]⁸⁺, 979 [M+9]⁹⁺, 881 [M+10]¹⁰⁺

LC-MS r.t. (min): 1.75 (LC-MS method 4A)

### DBCO-(M23D)₂

To a solution of M23D-mTz (16.4 mg, 1.86 μmol) in water (1.00 ml) and acetonitrile (0.400 ml) was added a stock solution of (1S,4E)-cyclooct-4-en-1-yl N-[2-(4-{2-azatricyclo[10.4.0.0^{4,9}]hexadeca-1(12),4(9),5,7,13,15-hexaen-10-yn-2-yl}-N-[2-({[(1S,4E)-cyclooct-4-en-1-yloxy]carbonyl}amino)ethyl]-4-oxobutanamido)ethyl]carbamate (0.67 mg, 0.964 μmol) in acetonitrile (675 μl) and the reaction mixture was stirred at room temperature. After every addition, the reaction progress was monitored with LC-MS^{3A}. In this way, complete conversion to the title product was obtained. The addition of the stock solution was as following; 400 μl, after 10 min—100 μl, after 10 min—100 μl, after 10 min—25 μl, after 10 min—25 μl, after 10 min—25 μl. Next, the reaction mixture was submitted to preparative LC-MS. Fractions corresponding to the product were immediately pooled together, frozen and lyophilized overnight to give the title compound (10.5 mg, 62%) as a white solid. Purity based on LC-MS 100% (broad peak).

LRMS (m/z): 1142 [M+16]¹⁶⁺, 1075[M+17]¹⁷⁺, 1015 [M+18]¹⁸⁺, including multiple m/z values of known fragments

LC-MS r.t. (min): 2.84 (LC-MS method 4B)

Conjugation of Murine Anti-CD63 (mCD63) mAb to DBCO-(M23D)₂
1. Preparation of mCD63

mCD63 was buffer exchanged into TBS using a Vivaspin (50 kDa MWCO), to a final concentration of 10.0 mg/ml.

### 2. Modification of the Carbohydrate on Antibody-Fc Domain

The immobilized GlycINATOR™ column (from GlyCLICK™ Azide Activation Kit, Genovis) was equilibrated and prepared according to the vendor's indications. The sample was then loaded on the column, the medium was resuspended, and the mixture was incubated and mixed at RT for 1 h. The column was then centrifuged, and the sample eluted.

UDP-GalNAz (from GlyCLICK™ Azide Activation Kit, Genovis) was reconstituted with TBS according to the vendor's indications and transferred to the pooled eluate together with GalT (from GlyCLICK™ Azide Activation Kit, Genovis). The mixture was incubated overnight, in the dark, at 30° C. Afterwards, the reaction was loaded onto a pre-conditioned desalting column (according to the indications of the vendor) and centrifuged to collect the flow-through, containing the azido-modified mAb. This was stored in the dark at 4 C, until later use for conjugation.

3. Conjugation with DBCO-(M23D)₂

mCD63 was buffer exchanged into DPBS using a Vivaspin (50 kDa MWCO) to a final concentration of 10.0 mg/ml DBCO-(M23D)₂ (5.0 equi., 1 mM in DPBS, pH 7.4) and was added to the mAb solution. The reaction mixture was incubated for 24 h at 37° C., then directly purified by preparative SEC (HiLoad 26/600 Superdex 200 μg, DPBS). The conjugate was characterized by SEC-UV (DAR determination). The pooled fractions were concentrated using the aforementioned Vivaspin to a final concentration of >10.0 mg/ml, sterile filtered over 0.22 μm filter units, and stored at 4° C. until further use.

| Test | Result |
|---|---|
| SEC (% monomers) | 98.4 |
| LC-MS (MW) | N/A |
| HIC (average DAR) | N/A |
| LC-MS (average DAR) | N/A |
| UV (average DAR) | 3.42 |
| Endotoxin (EU/mg) | 0.517 |
| Volume (ml) | 5.0 |
| Concentration (mg/ml) | 12.24 |
| Amount (mg) | 61.2 |
| Yield (%) | 76.5 |

Conjugation of Murine Anti-CD71 (mCD71) mAb to DBCO-(M23D)₂
1. Preparation of mCD71

mCD71 was buffer exchanged into TBS using a Vivaspin (50 kDa MWCO), to a final concentration of 10.0 mg/ml.

### 2. Modification of the Carbohydrate on Antibody-Fc Domain

The immobilized GlycINATOR™ column (from GlyCLICK™ Azide Activation Kit, Genovis) was equilibrated and prepared according to the vendor's indications. The sample was then loaded on the column, the medium was resuspended, and the mixture was incubated and mixed at RT for 1 h. The column was then centrifuged, and the sample eluted.

UDP-GalNAz (from GlyCLICK™ Azide Activation Kit, Genovis) was reconstituted with TBS according to the vendor's indications and transferred to the pooled eluate together with GalT (from GlyCLICK™ Azide Activation Kit, Genovis). The mixture was incubated overnight, in the dark, at 30° C. Afterwards, the reaction was loaded onto a pre-conditioned desalting column (according to the instructions of the vendor) and centrifuged to collect the flow-through, containing the azido-modified mCD71. This was stored in the dark at 4° C., until later use for conjugation.

3. Conjugation with DBCO-(M23D)₂

mCD71 was buffer exchanged into DPBS using a Vivaspin (50 kDa MWCO) to a final concentration of 10.0 mg/ml. DBCO-(M23D)₂ (5.0 equi., 1 mM in DPBS, pH 7.4) was added to the mCD71 solution, and the reaction mixture was incubated for 24 h at 37° C., then directly purified by preparative SEC (HiLoad 26/600 Superdex 200 μg, DPBS). The conjugate was characterized by SEC-UV (DAR determination). The pooled fractions were concentrated using the aforementioned Vivaspin to a final concentration of >10.0 mg/ml, sterile filtered over 0.22 μm filter units, and stored at 4° C. until further use.

| Test | Result |
|---|---|
| SEC (% monomers) | 99.8 |
| LC-MS (MW) | N/A |
| HIC (average DAR) | N/A |
| LC-MS (average DAR) | N/A |
| UV (average DAR) | 3.49 |
| Endotoxin (EU/mg) | 1.418 |
| Volume (ml) | 4.6 |
| Concentration (mg/ml) | 10.76 |
| Amount (mg) | 49.54 |
| Yield (%) | 69.7 |

### Cell Culture and In Vitro Experiments (Murine)

Murine myoblast cell line C2C12 was maintained in 10% FBS DMEM medium+Pen/Strep and plated at 240,000 cells per well (cpw) in 24-well plates or at 40,000 cpw in 96-well plates (wp), in maintenance medium (10% FBS in DMEM medium+Pen/Strep) and incubated at 37° C. with 5% CO₂. Twenty-four hours after seeding, cells were switched to differentiation media (2% horse serum in DMEM) and incubated for 3 days before refreshing the medium. After another 24 hours, medium was refreshed again and compounds were added and incubated for 48 hours. At 48 hours total post treatment, cells in the 24-wp were harvested for exon skip analysis and the cell viability was assessed on the 96-wp.

### Exon Skip Analysis and Quantification (Murine In Vitro)

Performed as described above in the Methods (as performed in Examples 1-4).

### Cell Viability (Murine In Vitro)

Performed as described above in the Methods (as performed in Examples 1-4).

### Methods for Example 6

### SO1861-SC-Maleimide

SO1861 was from Saponaria officinalis L (Extrasynthese, France) and was coupled to respective handles by Symeres (NL) according to methods known in the art.

### Conjugation of 5′-thiol-DMD-ASO, 5′-disulfidamide-DMD-PMO(1), and 3′-disulfidamide-DMD-PMO(1, 2, 3, 4 and 5) to antibodies

Custom conjugate productions of hCD71-5′-SS-DMD-ASO, hCD71-5′-SS-DMD-PMO(1), hCD71-3′-SS-DMD-PMO(1), hCD71-3′-SS-DMD-PMO(2), hCD71-3′-SS-DMD-PMO(3), hCD71-3′-SS-DMD-PMO(4), and hCD71-3′-SS-DMD-PMO(5) were performed by Fleet Bioprocessing (UK).

### UV-Vis Spectrophotometry

Concentrations were determined using either a Thermo Nanodrop 2000 spectrometer or Perkin Elmer Lambda 365 Spectrophotometer with the following mass Extinction Coefficient (EC) values:
Experimentally determined molar ε495=58,700 M-1 cm-1 and Rz280:495=0.428 were used for SAMSA-fluorescein.
Ellman's reagent (TNB); molar ε412=14,150 M-1 cm-1
Pyridine 2-thione (PDT); molar ε363=8,080 M-1 cm-1

Additionally, the DMD oligonucleotide incorporation was determined by UV-vis spectrophotometry and BCA colorimetric assay using literature ε²⁶⁵ values:
DMD-PMO(1); molar ε363=318,050 (mg/ml)-1 cm-1
DMD-PMO(2); molar ε363=318,120 (mg/ml)-1 cm-1
DMD-PMO(3); molar ε363=308,180 (mg/ml)-1 cm-1
DMD-PMO(4); molar ε363=247,710 (mg/ml)-1 cm-1
DMD-PMO(5); molar ε363=207,890 (mg/ml)-1 cm-1
DMD-ASO; molar ε363=310,00 (mg/ml)-1 cm-1

### SEC

The conjugates were analysed by SEC using an Akta purifier 10 system and Biosep SEC-s3000 column eluting with DPBS:IPA (85:15). Conjugate purity was determined by integration of the conjugate peak with respect to impurities/aggregate forms.

### SDS-PAGE

Native proteins and conjugates were analysed under heat denaturing non-reducing and reducing conditions by SDS-PAGE against a protein ladder using a 4-12% bis-tris gel and MOPS as running buffer (200 V, ˜40 minutes). Samples were prepared to 0.5 mg/ml, comprising LDS sample buffer and MOPS running buffer as diluent. For reducing samples, DTT was added to a final concentration of 50 mM. Samples were heat treated for 15 minutes at 90-95° C. and 2.5 μg (5 μL) was added to each well. Protein ladder (10 μL) was loaded without pre-treatment. Empty lines were filled with 1×LDS sample buffer (10 μL). After the gel was run, it was washed thrice with DI water (100 ml) with shaking (15 minutes, 200 rpm). Coomassie staining was performed by shaker-incubating the gel with PAGEBlue protein stain (30 ml) (60 minutes, 200 rpm). Excess staining solution was removed, rinsed twice with DI water (100 ml) and de-stained with DI water (100 ml) (60 minutes, 200 rpm). The resulting gel was imaged and processed using ImageJ (ImageJ (Rasband, W. S., ImageJ, U. S. National Institutes of Health, Bethesda, Maryland, USA) and MyCurveFit (point-to-point correlation of protein ladder).

### Western Blotting

From SDS-PAGE, the gel was transferred to nitrocellulose membrane using the X-Cell blot module with the following setup ((−)BP-BP-FP-Gel-NC-BP-BP-BP(+)) and conditions (30V, 60 minutes) using freshly prepared transfer buffer. BP—blotting pad; FP—Filter pad; NC—Nitrocellulose membrane. After, the NC were washed thrice with PBS-T (100 ml) with shaking (5 minutes, 200 rpm), non-specific sites blocked with blocking buffer (50 ml) with shaking (30 minutes, 200 rpm) then active sites labelled with a combination of Goat anti-Human Kappa—HRP (1:2000) and Goat anti-Human IgG—HRP (1:2000) (50 ml) diluted in blocking buffer with shaking (30 minutes, 200 rpm). After that, the NC was washed once with PBS-T (100 ml) with shaking (5 minutes, 200 rpm) and complexed antibody detected with Ultra TMB-Blotting Solution (25 ml). Colour development was observed visually, and development was stopped by washing the NC with water, and the resulting blot was photographed.

### Urea-PAGE Gel Electrophoresis

This characterisation of conjugates was carried out under denaturing non-reducing conditions with EtBr or SYBR Green staining, compared against oligonucleotide standards and oligonucleotide standards ladder (report residual ‘free’ oligonucleotide).

hAb-DMD-Oligonucleotide

A general description of the conjugation of hAb targeted DMD oligonucleotides, such as hCD71-5′-SS-DMD-ASO, hCD71-5′-SS-DMD-PMO(1), and hCD71-3′-SS-DMD-PMO(1-5), is shown below. The quantities given in brackets and italics are shown for hCD71-5′-SS-DMD-PMO(1), as an example.

An aliquot of hAb was buffer exchanged into DPBS pH 7.5 and normalized to 2.5 mg/ml. To an aliquot of hAb (hCD71, 20.0 mg, 1.33×10-4 mmol, 2.5 mg/ml) was added an aliquot of freshly prepared PEG4-SPDP solution (10.0 mg/ml, 10.0 mole equivalents, 1.33×10-3 mmol, 0.075 ml), the mixture was vortexed briefly and then incubated for 60 minutes at 20° C. with roller-mixing. After incubation, the reaction was quenched by the addition of an aliquot of a freshly prepared glycine solution (10 mg/ml, 50 mole equivalents, 6.65×10-3 mmol, 8.1 μl), the mixture vortexed briefly, then incubated for >15 minutes at 20° C. with roller-mixing. The conjugate was purified (using Zeba 40K spin desalting columns eluting with TBS pH 7.5, filtered to 0.45 μm), then analysed by UV-vis to give purified hAb-SPDP (hCD71-SPDP, 21.2 mg, 2.04 mg/ml, SPDP to hCD71 ratio=3.9). hAb-SPDP was used immediately.

Separately, the desired DMD oligonucleotide (DMD-PMO(1)-5′-amide, 20.2 mg, 1.99×10-3 mmol, 10.00 mg/m/l) was reconstituted using TBS pH 7.5, pooled into a single aliquot and analysed by UV-vis to ascertain ε₂₈₀, ε₂₆₀ and their ratio ε₂₆₀/ε₂₈₀. To this was added an aliquot of freshly prepared THPP solution (50 mg/ml, 10 mole equivalents, 1.99×10-2 mmol, 83 μl), the mixture was vortexed briefly, then incubated for 60 minutes at 37° C. with roller-mixing. After incubation, the oligonucleotide was purified using PD10 Sephadex G25 columns eluting with TBS pH 7.5, to afford the reduced DMD oligonucleotide-SH (DMD-PMO(1)-5′-SH, 16.4 mg, 81%, thiol to DMD-PMO(1) ratio=1.02).

To an aliquot of hAb-SPDP (hCD71-SPDP, 10.2 mg, 6.79×10-5 mmol, 2.04 mg/m) was added an aliquot of oligonucleotide-SH (DMD-PMO(1)-SH, 2.73 mg/ml, 8.0 mole equivalents, 5.43×10-4 mmol, 2.01 ml), the mixture was vortexed briefly and then incubated overnight at 20° C. with roller-mixing. After ca. 16 hours, the conjugate mixture was analysed by UV-vis to ascertain incorporation by PDT displacement and then purified using a sanitized 2.6×60 cm Superdex 200PG column eluting with DPBS pH 7.5. The conjugate was analysed by UV-vis and BCA colorimetric assay and assigned a new ε₂₈₀. The material was concentrated (using a Vivacell 100 (30 K MWCO), to the maximum concentration obtainable) and then dispensed into aliquots for product testing and characterisation. The result was a hCD71-5′-SS-DMD-PMO(1) conjugate (total yield=65%; DMD-PMO(1) to hCD71 ratio=2.2), as an example. The other results were:
hCD71-3′-SS-DMD-PMO(1) (total yield=58%, DMD-PMO(1) to hCD71 ratio=2.1)
hCD71-3′-SS-DMD-PMO(2) (total yield=70%, DMD-PMO(2) to hCD71 ratio=3.0)
hCD71-3′-SS-DMD-PMO(3) (total yield=65%, DMD-PMO(3) to hCD71 ratio=2.6)
hCD71-3′-SS-DMD-PMO(4) (total yield=68%, DMD-PMO(4) to hCD71 ratio=2.3)
hCD71-3′-SS-DMD-PMO(5) (total yield=67%, DMD-PMO(5) to hCD71 ratio=2.1)
hCD71-5′-SS-DMD-ASO (total yield=76%, DMD-ASO to hCD71 ratio=2.1)

### Cell Culture (Human)

Immortalized human myoblasts from non-DMD donors (KM155) were cultured as described above in the Methods (as performed in Examples 1-4).

### Exon Skip Analysis and Quantification (Human)

RNA was isolated with the TRIsure Isolation Reagent (Bioline) and chloroform extraction; isopropanol precipitation of RNA from the aqueous phase was performed as known to someone skilled in the art. For cDNA synthesis, 1000 ng of total RNA was used and diluted in an appropriate amount of RNase-free water to yield 8 μl RNA dilution. The priming premix contained 1 μl dNTP mix (10 mM each) and 1 μl specific reverse primer (for KM155, exon 51: h53R 5′-CTCCGGTTCTGAAGGTGTTC-3′ [SEQ ID NO: 5]; exon 53: h55R 5′-ATCCTGTAGGACATTGGCAGTT-3 [SEQ ID NO: 6]. This mixture was heated for 5 min at 70° C., then chilled on ice for at least 1 min. A reaction mixture was prepared containing 0.5 μl rRNasin (Promega), 4.0 μl 5×RT buffer (Promega), 1.0 μl M-MLV RT (Promega), and 4.5 μl RNase-free water, and was added to the chilled mixture to yield a total volume of 20 μl per reaction. The RT-PCR was run for 60 min at 42° C., then 5 min at 85° C., and chilled on ice. For skip analysis, a nested PCR approach was followed. To this end, in the first PCR, 3 μl cDNA was added to a mix of 2.5 μl 10× SuperTaq PCR buffer, 0.5 μl dNTP mix (10 mM each), 0.125 μl Taq DNA polymerase TAQ-RO (5 U/μl; Roche), 16.875 μl RNase-free water and 1 μl (10 pmol/μl) of each primer flanking the targeted exons. The following primers were used: for KM155, exon 51: h48F 5′-AAAAGACCTTGGGCAGCTTG-3′ [SEQ ID NO: 7] and h53R 5′-CTCCGGTTCTGAAGGTGTTC-3′ [SEQ ID NO: 5]; exon 53: h50F 5′-AGGAAGTTAGAAGATCTGAGC-3′ [SEQ ID NO: 20] and h55R 5′-ATCCTGTAGGACATTGGCAGTT-3′ [SEQ ID NO: 6]. These samples were subjected to a PCR run of 5 min at 94° C., then 25 cycles with 40 sec at 94° C., 40 sec at 60° C., 180 sec at 72° C., after which for 7 min at 72° C. For the second PCR, 1.5 μl PCR1 sample was added to a mix of 5 μl 10× SuperTaq PCR buffer, 1 μl dNTP mix (10 mM each), 0.25 μl Taq DNA polymerase TAQ-RO (5 U/μl; Roche), 38.25 μl RNase-free water and 2 μl (10 pmol/μl) of each primer flanking the targeted exons. The following primers were used: for KM155, exon 51: h49F 5′-CCAGCCACTCAGCCAGTG-3′ [SEQ ID NO: 10] and h52R2 5′-TTCTTCCAACTGGGGACGC-3′ [SEQ ID NO: 11], exon 53: h52F 5′-CCCCAGTTGGAAGAACTCATT-3′ [SEQ ID NO: 21] and h54R 5′-CCAAGAGGCATTGATATTCTC-3′ [SEQ ID NO: 9]. These samples were subjected to a PCR run of 5 min at 94° C., then 32 cycles with 40 sec at 94° C., 40 sec at 60° C., 60 sec at 72° C., after which for 7 min at 72° C. Exon skipping levels were quantified using the Femto Pulse System using the Ultra Sensitivity NGS Kit (Agilent), according to the manufacturer's instructions. Alternatively, the specific PCR fragments were analysed using Bioanalyzer 2100 with DNA1000 chip (lab-on-a-chip; Agilent). For exon 51 skipping, the expected non-skipped product has a size of 408 bp (KM155) and the skip product of 175 bp (KM155). For exon 53 skipping, the expected non-skipped product has a size of 438 bp (KM155) and the skip product of 226 bp (KM155).

### Results

### Example 1. DMD-PMO+SO1861 and DMD-ASO+SO1861

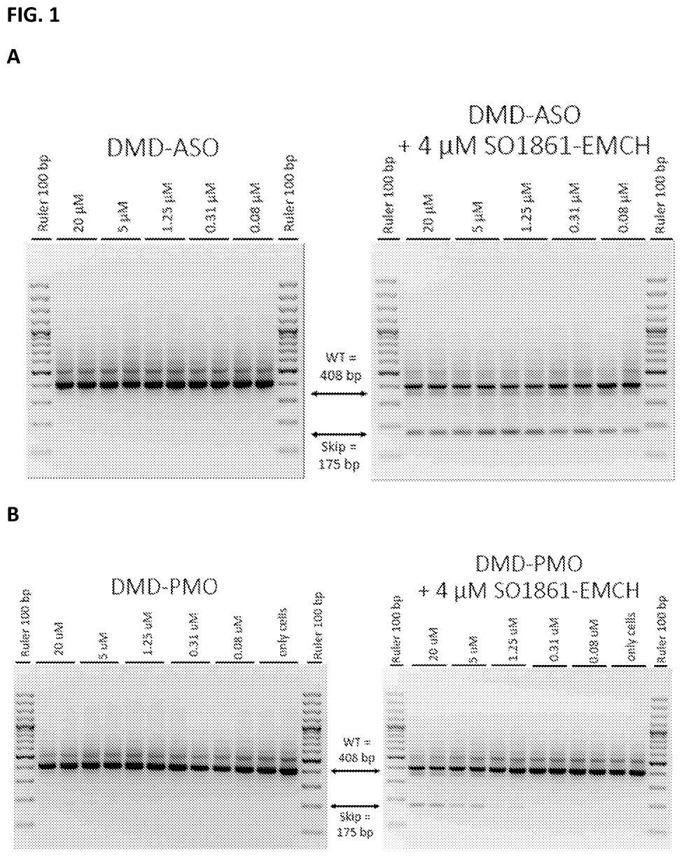

DMD-ASO (a 2′O-methyl-phosporothioate antisense oligonucleotide that induces exon 51 skipping of human dystrophin and has the same sequence and chemistry modifications as drisapersen) and DMD-PMO (a phosphorodiamidate morpholino oligomer antisense oligonucleotide that induces exon 51 skipping of human dystrophin and has the same sequence but not 5′-modifications as eteplirsen) were assessed for exon skipping activity in combination with the endosomal escape enhancer SO1861-EMCH (4 μM) in differentiated human myotubes derived from a non-DMD (healthy) donor (KM155). Surprisingly, this revealed strongly enhanced exon skipping of exon 51 only in combination with SO1861 after 72 hrs (Table A3): while exposure to 20 μM DMD-ASO alone showed no skipping activity, already 0.08 μM DMD-ASO+SO1861-EMCH revealed % exon skip, indicating that the improvement factor is exceeding 250-fold (FIG. 1A). Likewise, exposure to 20 μM DMD-PMO resulted in 4% skip, while already 1.25 μM DMD-ASO+SO1861-EMCH achieved 5% skip, and 34% at 20 μM, indicating an increase in potency of 16-fold for the untargeted oligos with untargeted SO1861-EMCH (FIG. 1B).

In conclusion, co-administration of SO1861-EMCH strongly enhances the on-target delivery of DMD-ASO and DMD-PMO and induces marked exon 51 skipping at one to two orders of magnitude lower exposure concentrations compared to conditions where SO1861-EMCH it is not present.

TABLE A3
Skip efficacy of MD oligos with and without SO1861-
EMCH co-administration in human myotubes (KM155)

| % exon skipping | | Concentration DMD oligo | | | | |
|---|---|---|---|---|---|---|
| (Lab-on-a-chip or Femto analysis) | | 20 μM | 5 μM | 1.25 μM | 0.31 μM | 0.08 μM |
| DMD-ASO | Lab-on-a-chip | 1% | 1% | 0% | 0% | 0% |
| DMD-ASO + | Lab-on-a-chip | 52% | 61% | 67% | 53% | 40% |
| SO1861-EMCH | | | | | | |
| DMD-PMO | Femto | 4% | 0% | 0% | 0% | 0% |
| DMD-PMO + | Femto | 34% | 19% | 5% | 0% | 0% |
| SO1861-EMCH | | | | | | |

### Example 2. hCD71-DMD-ASO+SO1861-EMCH or hCD71-DMD-PMO+SO1861-EMCH or mCD71-M23D+SO1861-EMCH

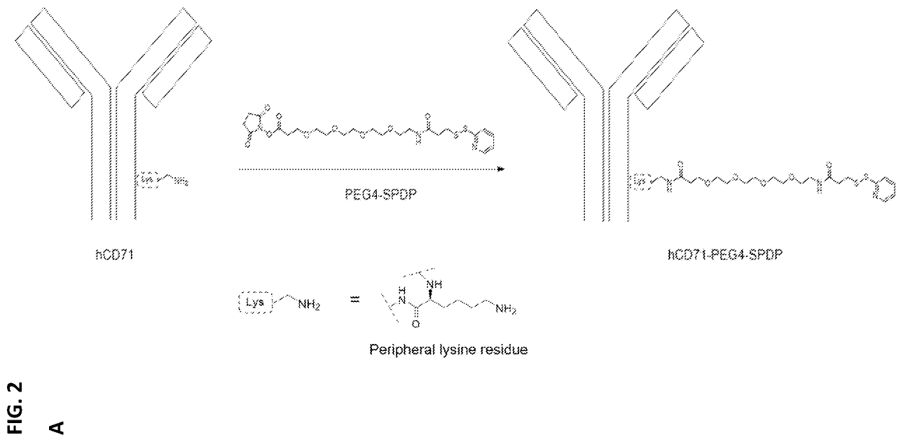

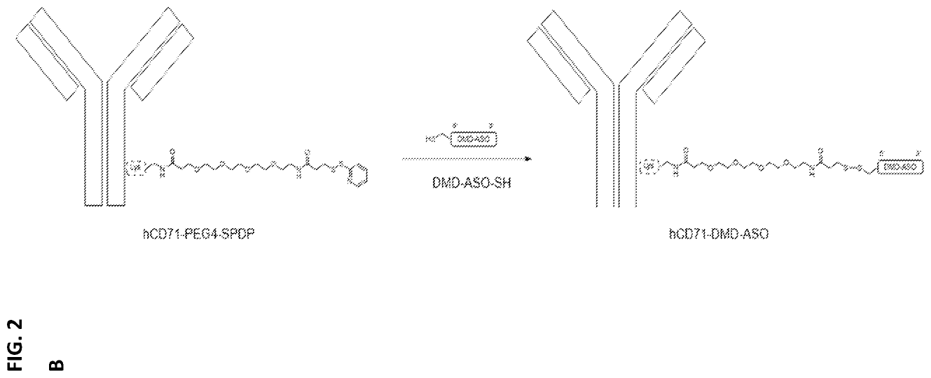

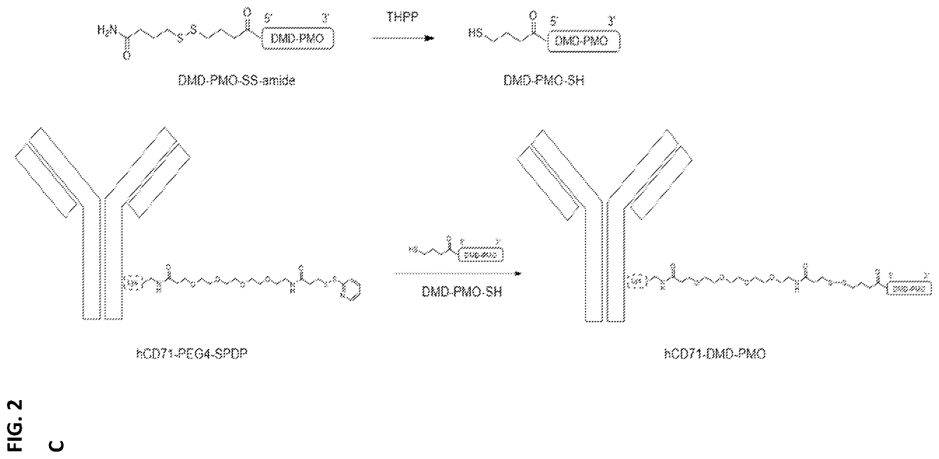

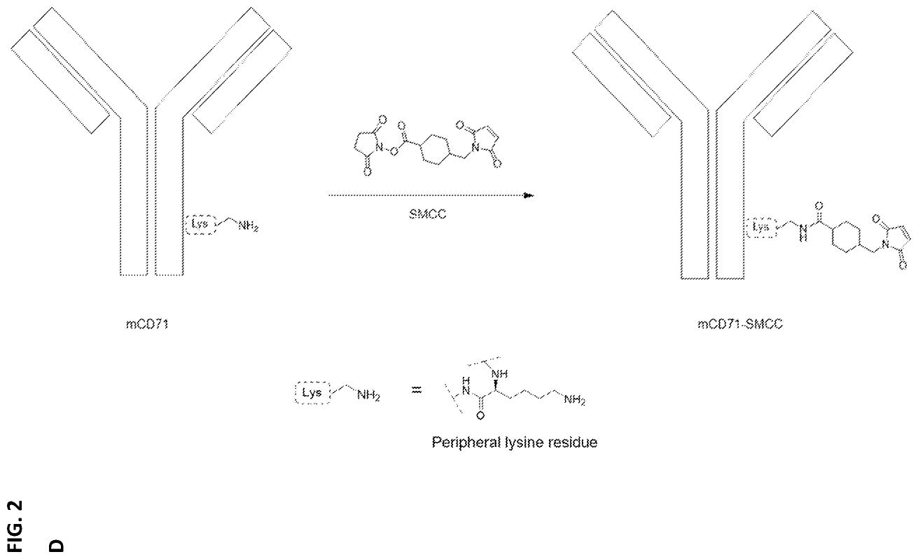

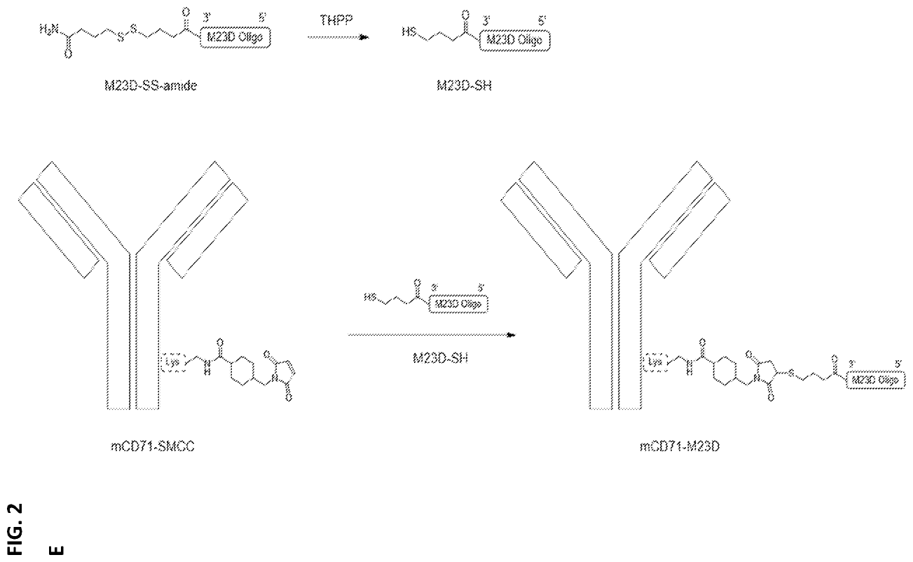

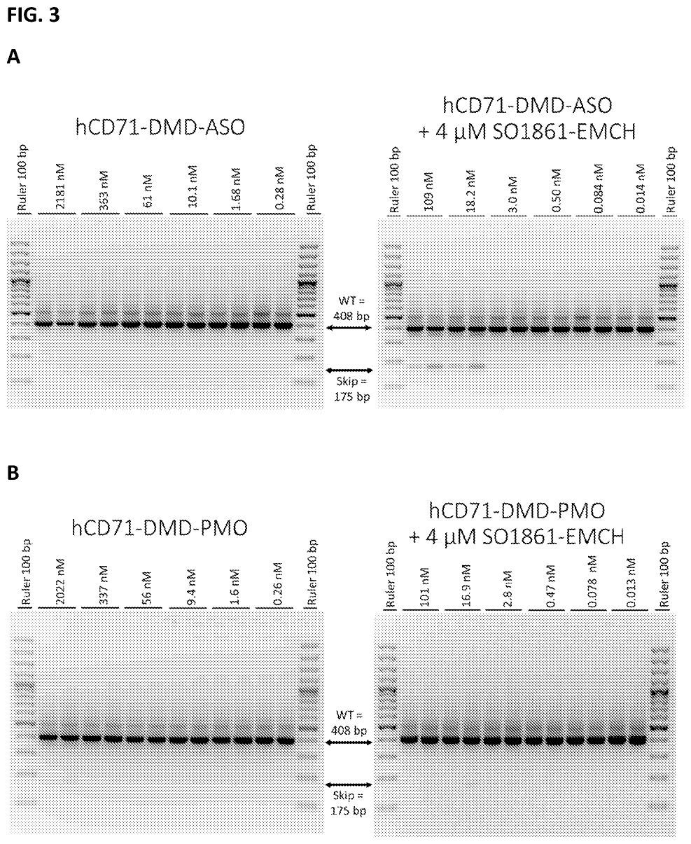

DMD-ASO-SH and DMD-PMO-SH were conjugated as shown in FIG. 2B and FIG. 2C, respectively to PEG4-SPDP-modified human anti-CD71 monoclonal antibody (hCD71-PEG4-SPDP, FIG. 2A) to produce: hCD71-DMD-ASO (DAR2.1) and hCD71-DMD-PMO (DAR3.2). The resultant compounds were tested for enhanced cytoplasmic DMD oligo delivery and enhanced dystrophin exon 51 skipping either without or in combination with 4 μM SO1861-EMCH on the differentiated human myotubes from a non-DMD donor (KM155) and a DMD-affected donor (DM8036). This revealed strongly enhanced exon skipping of exon 51 in combination with 4 μM SO1861-EMCH at markedly lower concentrations compared to hCD71-DMD-ASO alone, and to hCD71-DMD-PMO alone, respectively after 72 hrs of treatment (Table A4 and A5): hCD71-DMD-ASO resulted in low skip (3%) in KM155 at 2181 nM (FIG. 3A, left panel), which is an improvement compared to non-targeted DMD-ASO (Table A3), while hCD71-DMD-ASO+SO1861 furthermore increased skip to 18-24% already at 18.2-109 nM in KM155 (FIG. 3A, right panel). Likewise, hCD71-DMD-PMO resulted in no skip at 2022 nM (0%) in KM155 (FIG. 3B, left panel), while with addition of SO1861-EMCH, exon skip was visible at a concentration of 2.8-16.9 nM hCD71-DMD-PMO (FIG. 3B, right panel). More importantly, in differentiated myotubes from a DMD-affected donor (DM8036), only 17% exon 51 skip was observed at 333 nM hCD71-DMD-ASO (FIG. 4A, left panel), while, surprisingly, the 720-fold lower concentration of 0.50 nM hCD71-DMD-ASO+4 μM SO1861-EMCH already resulted in comparable skip (15%). The skip efficiency strikingly increased up to 64-68% at only 18.2-109 nM exposure concentration of hCD71-DMD-ASO (FIG. 4A, right panel). Likewise, also in DM8036 myotubes, hCD71-DMD-PMO alone reached 21% skip at 337 nM (FIG. 4B, left panel), whereas already an exposure concentration of 0.08-0.47 nM hCD71-DMD-PMO+4 μM SO1861-EMCH resulted in clearly measurable skip (7-13%), which increased up to 33% at 101 nM (FIG. 4B, right panel) realizing an improvement of one to two orders of magnitude.

Taken together, this shows that co-administration of SO1861-EMCH to targeted oligonucleotides hCD71-DMD-ASO and hCD71-DMD-PMO markedly improves on-target cytoplasmic delivery and specifically, in relevant cell systems such as differentiated myotubes from DMD-affected donors.

TABLE A4
Skip efficacy of anti-CD71-conjugated DMD oligos
in human myotubes (top concentration for DMD-
ASO-conjugate, bottom for DMD-PMO-conjugate)

| | [nM conjugate] | | | | | | |
|---|---|---|---|---|---|---|---|
| % exon skipping | 2181/ | 363/ | 61/ | 10.1/ | 1.68/ | 0.28/ | |
| (Femto analysis) | 2022 | 337 | 56 | 9.4 | 1.56 | 0.26 | |
| hCD71-DMD-ASO | 3 | 0 | 0 | 0 | 0 | 0 | % DMD |
| (DAR2.1) (KM155) | | | | | | | exon 51 |
| hCD71-DMD-PMO | 0 | 0 | 0 | 0 | 0 | 0 | skipping |
| (DAR3.2) (KM155) | | | | | | | |
| hCD71-DMD-ASO | 24 | 17 | 0 | 0 | 0 | 0 | |
| (DAR2.1) | | | | | | | |
| (DM8036) | | | | | | | |
| hCD71-DMD-PMO | 45 | 21 | 0 | 0 | 0 | 0 | |
| (DAR3.2) | | | | | | | |
| (DM8036) | | | | | | | |

TABLE A5
Skip efficacy of anti-CD71-conjugated DMD oligos with SO1861-
EMCH co-administration in human myotubes (top concentration
for DMD-ASO-conjugate, bottom for DMD-PMO-conjugate)

| | [nM conjugate] | | | | | | |
|---|---|---|---|---|---|---|---|
| % exon skipping | 109/ | 18.2/ | 3.0/ | 0.50/ | 0.084/ | 0.014/ | |
| (Femto analysis) | 101 | 16.9 | 2.8 | 0.47 | 0.078 | 0.013 | |
| hCD71-DMD- | 18 | 24 | 0 | 0 | 0 | 0 | % DMD |
| ASO(DAR2.1) + | | | | | | | exon 51 |
| SO1861-EMCH (KM155) | | | | | | | skipping |
| hCD71-DMD-PMO | 0 | 3 | 2 | 0 | 0 | 0 | |
| (DAR3.2) + | | | | | | | |
| SO1861-EMCH (KM155) | | | | | | | |
| hCD71-DMD-ASO | 64 | 68 | 62 | 15 | 1 | 0 | |
| (DAR2.1) + | | | | | | | |
| SO1861-EMCH (DM8036) | | | | | | | |
| hCD71-DMD-PMO | 33 | 34 | 15 | 13 | 7 | 0 | |
| (DAR3.2) + | | | | | | | |
| SO1861-EMCH (DM8036) | | | | | | | |

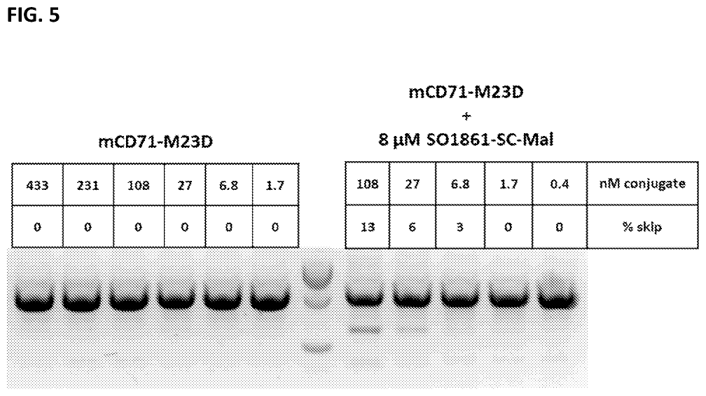

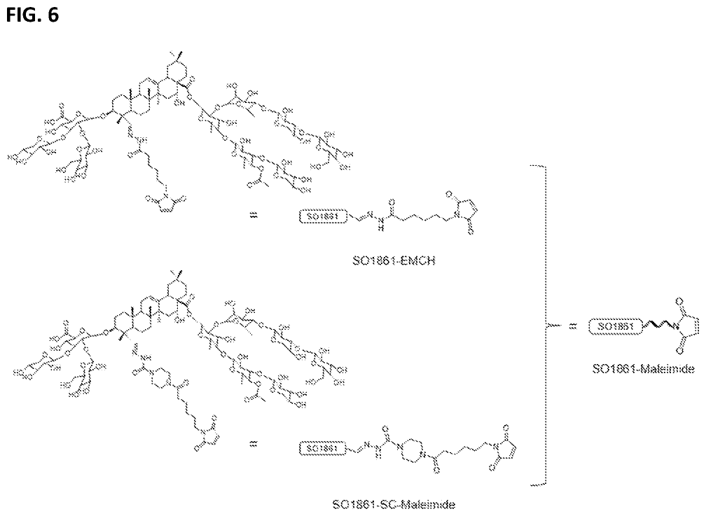

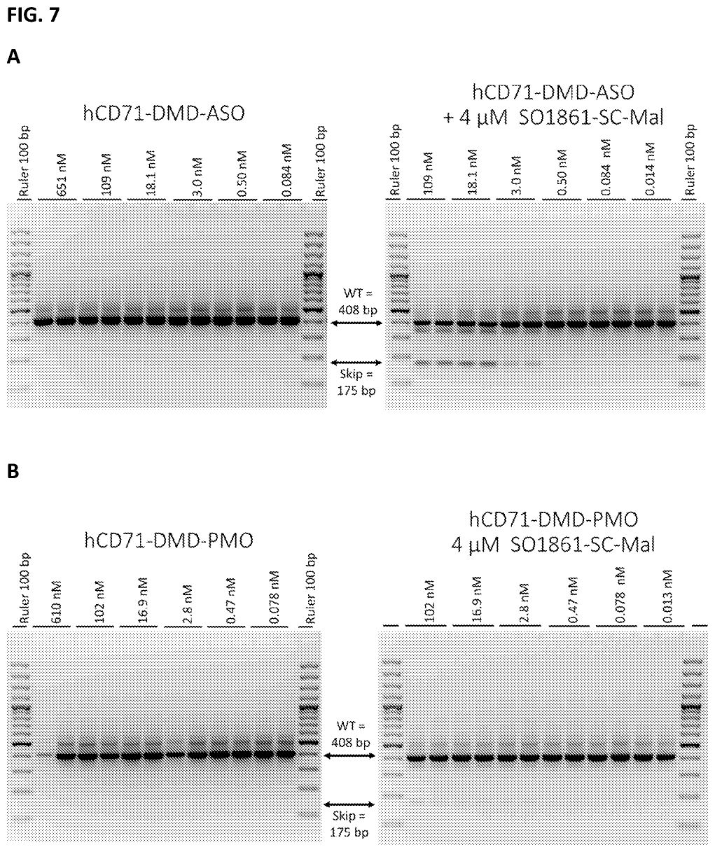

Next, M23D-SS-amide (a phosphorodiamidate morpholino oligomer antisense oligonucleotide that induces exon 23 skipping of mouse dystrophin) was conjugated to anti-CD71 monoclonal antibody targeting murine CD71 modified with SMCC-linker (mCD71-SMCC, FIG. 20) to produce mCD71-M23D (DAR1.2) (FIG. 2E). Co-administration of mCD71-M23D+8 μM SO1861-SC-Mal was tested on differentiated C2C12 myotubes and this revealed strong exon skipping enhancement with at least one order of magnitude lower concentrations mCD71-M23D (clear band until 27 nM, FIG. 5, right panel) in the combination with SO1861-SC-Mal, whereas mCD71-M23D alone showed no activity at all concentration tested up to 433 nM (FIG. 5, left panel).

This shows that co-administration of SO1861-compounds is broadly applicable cross-species to effectively increase potency of targeted PMOs with different targeting sequences (different exons, different sequences).

### Example 3. hCD71-DMD-ASO+SO1861-SC-Mal or hCD71-DMD-PMO+SO1861-SC-Mal

DMD-ASO-SH or DMD-PMO-SH were (as previously described in FIG. 2B and FIG. 20) conjugated to anti-CD71 monoclonal antibody targeting human 0071 (hCD71) to yield hCD71-DMD-ASO (DAR2.2) and hCD71-DMD-PMO (DAR3.1). Conjugates, including controls, were tested on differentiated human myotubes of a non-DMD donor (KM 155) and of a DM0-affected donor (DM8036). As expected, these treatments revealed no or only very minor exon skip in KM155 myotubes in the concentration range from 0.084 nM-651 nM for hCD71-DMD-ASO (FIG. 7A, left panel; Table A6) and from 0.078 nM-610 nM hCD71-DMD-PMO (FIG. 7B, left panel; Table A6) at 72 hrs post dose, ranging from 0-2.4%. However, when co-administering 4 μM SO1861-SC-Mal to either hCD71-DMD-ASO (FIG. 7A, right panel; Table A7) or to hCD71-DMD-PMO (FIG. 7B, right panel; Table A7), strongly enhanced exon skip was observed in differentiated human myotubes: already at ca 0.5 nM, 0-5% and 0-4% exon skip were observed for hCD71-DMD-ASO and hCD71-DMD-PMO, respectively. This increased to 29-40% at 109 nM hCD71-DMD-ASO and 4-5% at 102 nM hCD71-DMD-PMO, respectively, constituting a several order of magnitude improvement in potency, compared to conditions without SO1861-SC-Mal addition.

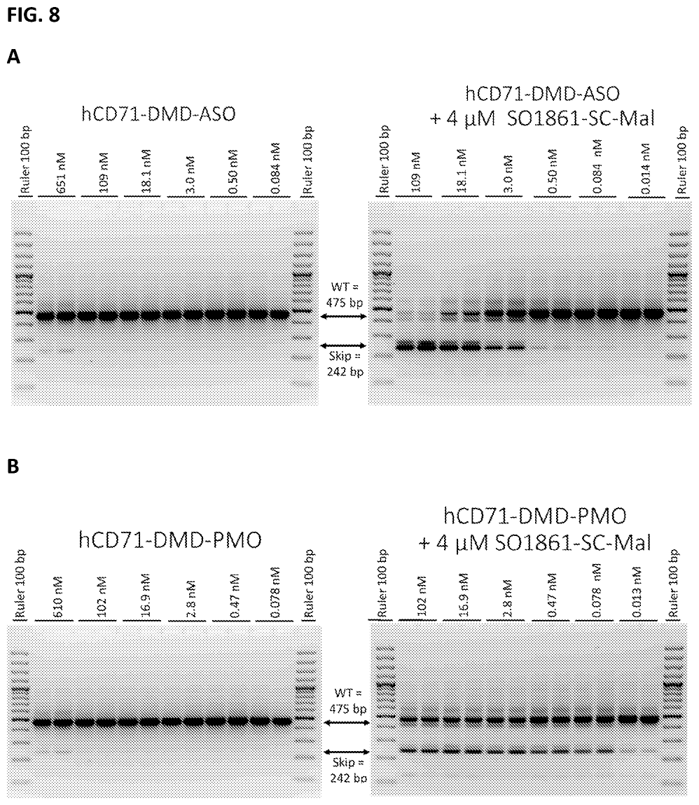

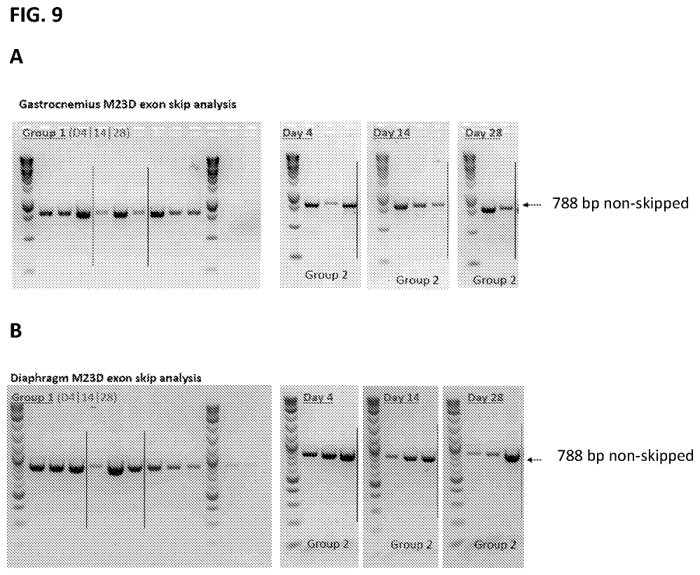

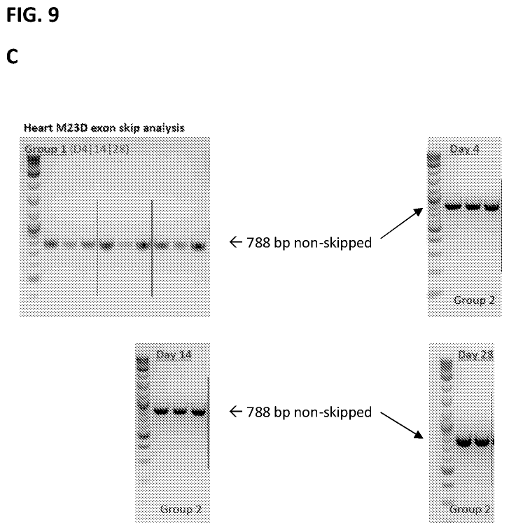

More relevantly, in differentiated myotubes from a DM0-affected donor, treatments with targeted conjugates hCD71-DMD-ASO and hCD71-DMD-PMO at 72 hrs post dose again revealed maximally 7-11% skip at 651 nM hCD71-DMD-ASO (FIG. 7A, left panel; Table A7) and 610 nM hCD71-DMD-PMO (FIG. 7B, left panel; Table A7), respectively. However, strikingly, and when co-administering 4 μM SO1861-SC-Mal to either hCD71-DMD-ASO or hCD71-DMD-PMO, strongly enhanced exon skip was observed in differentiated human myotubes from a DM0-affected donor: with 92-96% skip at 109 nM hCD71-DMD-ASO+SO1861-SC-Mal (FIG. 8A, right panel) and 37-57% skip at 102 nM hCD71-DM0-PMO+SO1861-SC-Mal (FIG. 8B, right panel). An effect was still measurable down to at least 0.50 nM hCD71-DMD-ASO, and even at 0.013 nM, skip was observed for hCD71-DMD-PM+S1861-SC-Mal.

TABLE A6
Skip efficacy of anti-CD71-conjugated DMD oligos in human myotubes (top
concentration for DMD-ASO-conjugate, bottom for DMD-PMO-conjugate)

| | | [nM conjugate] | | | | | | |
|---|---|---|---|---|---|---|---|---|
| Exon skip % | | 651/ | 109/ | 18.1/ | 3.0/ | 0.50/ | 0.084/ | |
| ImageJ and Femto analysis | | 610 | 102 | 16.9 | 2.8 | 0.47 | 0.078 | |
| hCD71-DMD-ASO | ImageJ | 1.9 | 0.0 | 0.5 | 1.0 | 1.1 | 1.5 | % DMD |
| (DAR2.2) (KM155) | Femto | 0.0 | 0.0 | 0.0 | 0.0 | 0.0 | 0.0 | exon 51 |
| hCD71-DMD-PMO | ImageJ | 0.3 | 1.4 | 2.4 | 2.0 | 1.8 | 2.0 | skipping |
| (DAR3.1) (KM155) | Femto | 0.0 | 0.0 | 0.0 | 0.0 | 0.0 | 0.0 | |
| hCD71-DMD-ASO | ImageJ | 6.9 | 1.6 | 1.4 | 1.0 | 1.3 | 1.4 | |
| (DAR2.2) (DM8036) | Femto | 11.1 | 0.0 | 0.0 | 0.0 | 0.0 | 0.0 | |
| hCD71-DMD-PMO | ImageJ | 7.3 | 3.5 | 3.2 | 2.8 | 3.3 | 2.2 | |
| (DAR3.1) (DM8036) | Femto | 7.9 | 0.0 | 0.0 | 0.0 | 0.0 | 0.0 | |

TABLE A7
Skip efficacy of anti-CD71-conjugated DMD oligos with
SO1861-SC-Mal co-administration in human myotubes
(top concentration for DMD-ASO, bottom for DMD-PMO)

| | | [nM conjugate] | | | | | | |
|---|---|---|---|---|---|---|---|---|
| Exon skip % | | 109/ | 18.1/ | 3.0/ | 0.50/ | 0.084/ | 0.014/ | |
| ImageJ and Femto analysis | | 102 | 16.9 | 2.8 | 0.47 | 0.078 | 0.013 | |
| hCD71-DMD-ASO | ImageJ | 28.8 | 29.4 | 13.9 | 5.1 | 3.0 | 3.0 | % DMD |
| (DAR2.2) + | Femto | 40.3 | 38.4 | 13.5 | 0.0 | 0.0 | 0.0 | exon 51 |
| SO1861-SC-Mal | | | | | | | | skipping |
| (KM155) | | | | | | | | |
| hCD71-DMD-PMO | ImageJ | 4.6 | 5.2 | 5.2 | 4.1 | 2.8 | 1.9 | |
| (DAR3.1) + | Femto | 3.9 | 4.4 | 2.0 | 0.0 | 0.0 | 0.0 | |
| SO1861-SC-Mal | | | | | | | | |
| (KM155) | | | | | | | | |
| hCD71-DMD-ASO | ImageJ | 92.2 | 56.6 | 34.1 | 7.0 | 2.2 | 0.0 | |
| (DAR2.2) + | Femto | 96.1 | 88.0 | 53.8 | 7.4 | 0.0 | 0.0 | |
| SO1861-SC-Mal | | | | | | | | |
| (DM8036) | | | | | | | | |
| hCD71-DMD-PMO | ImageJ | 37.1 | 38.3 | 34.7 | 28.9 | 25.2 | 6.1 | |
| (DAR3.1) + | Femto | 57.2 | 65.0 | 55.8 | 46.5 | 40.9 | 9.9 | |
| SO1861-SC-Mal | | | | | | | | |
| (DM8036) | | | | | | | | |

### Example 4. mCD71-M23D (In Vivo Efficacy)

CD-1 male mice received a single injection of mCD71-M23D (DAR1.2) (FIG. 2). Additionally, a vehicle control group was included.

As FIG. 9 (A-C) shows, animals receiving vehicle (group 1) or mCD71-M23D (2.80 mg/kg PMO; group 2) showed no exon 23 skip in any of the tested tissues or any time point (neither at day 4, day 14 nor day 28).

This data shows that conjugates without the presence of targeted SO1861 do not achieve any skip at tested doses.

In Vivo Tolerability of mCD71-M23D in CD-1 Mice

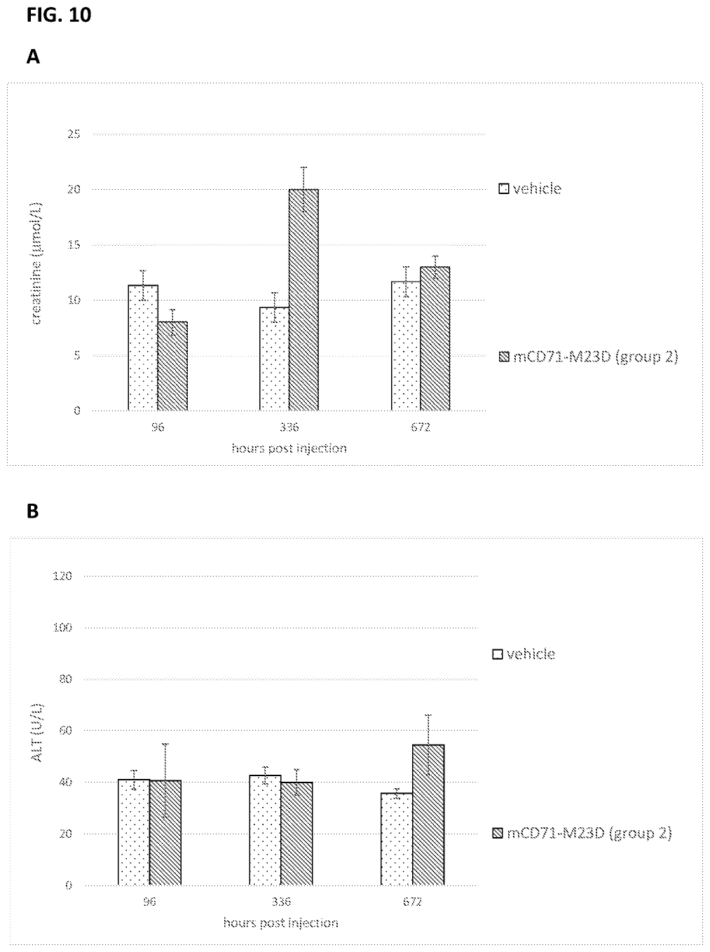

The conjugate mCD71-M23D (FIG. 2) was dosed as detailed in Table A2. During the course of the study, one animal treated with mCD71-M23D (of six remaining in group 2) was found dead on day 11, also resulting in missing biomarker data for one of three mice at day 28. At the day 14 sacrifice, two (of three) mice dosed with mCD71-M23D in group 2 showed kidney abnormalities and elevated serum creatinine (FIG. 10A). No marked or lasting changes in the kidney biomarker ALT were obvious (FIG. 10B). Notably, after 14 days and 28 days, ALT levels were comparable to vehicle controls (group 1).

### Example 5. mCD71-M23D+SO1861-SC-Mal or mCD63-M23D+SO1861-SC-Mal (In Vitro)

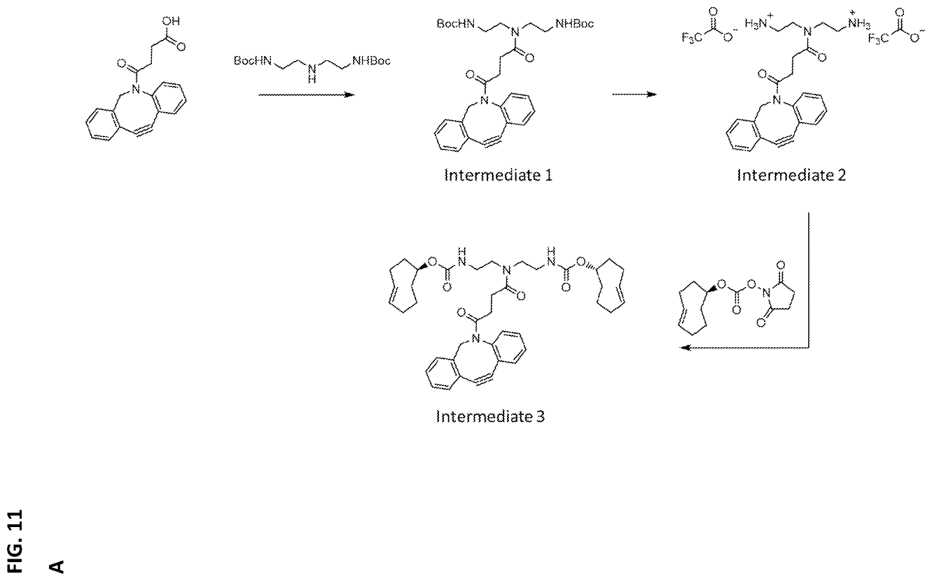

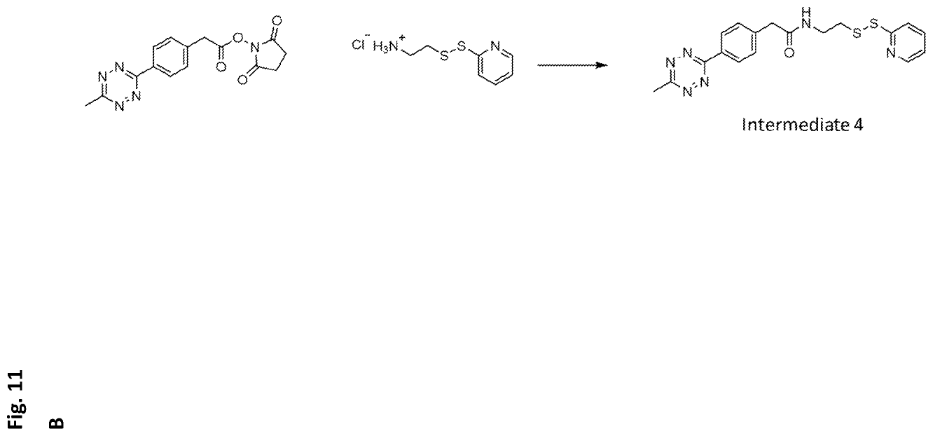

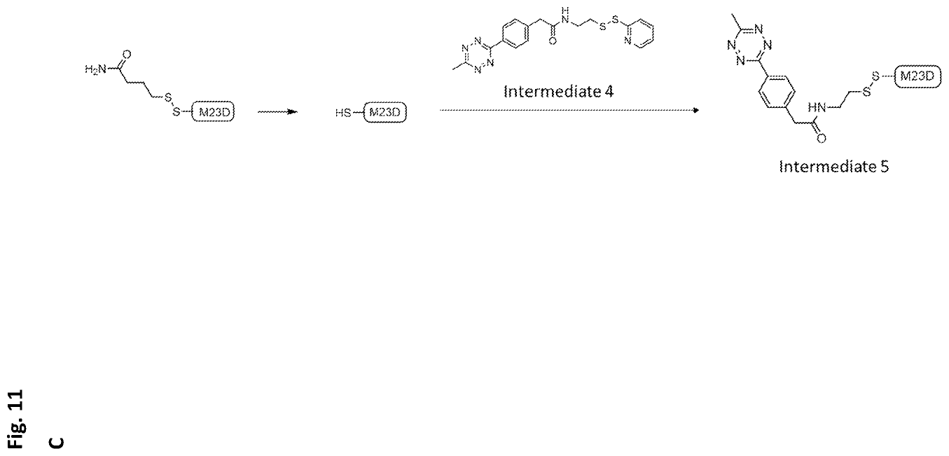

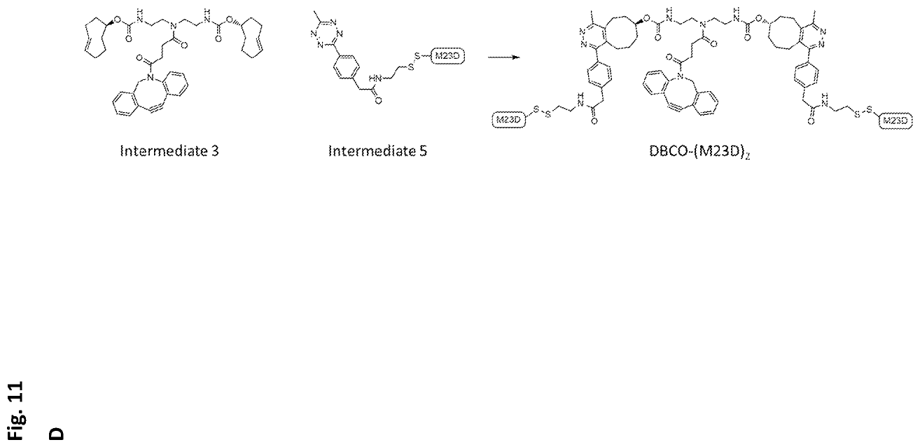

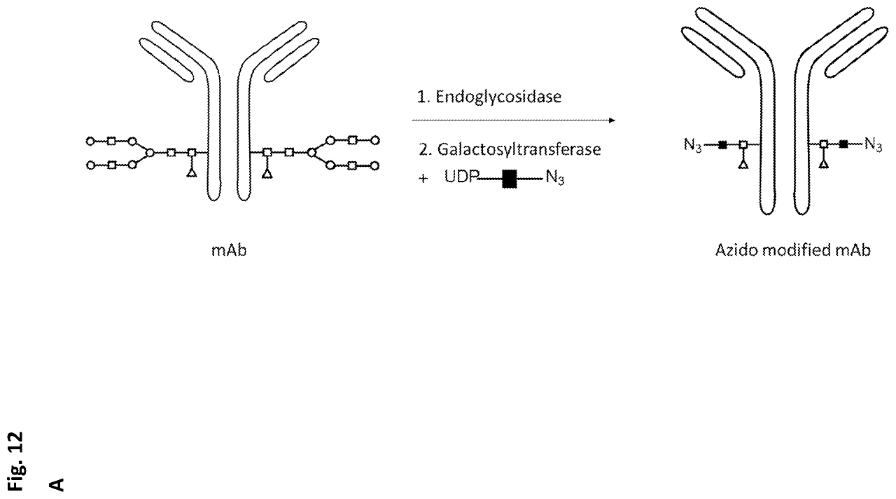

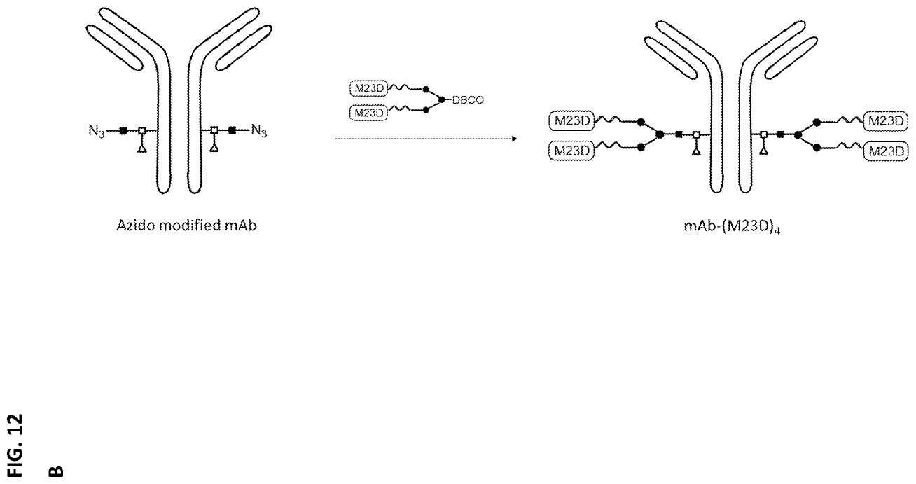

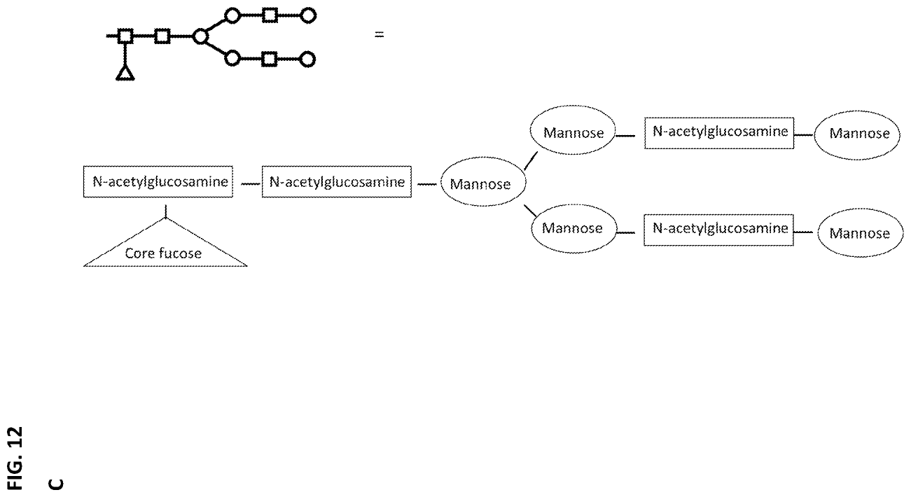

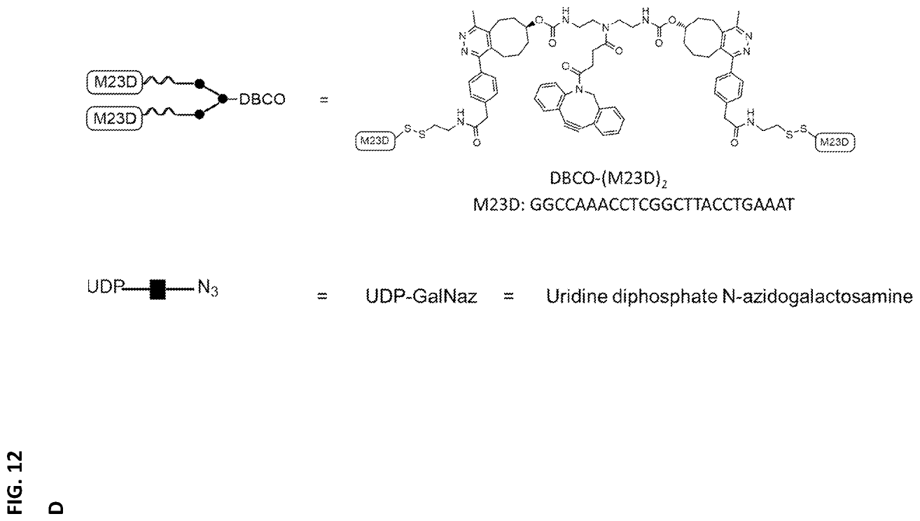

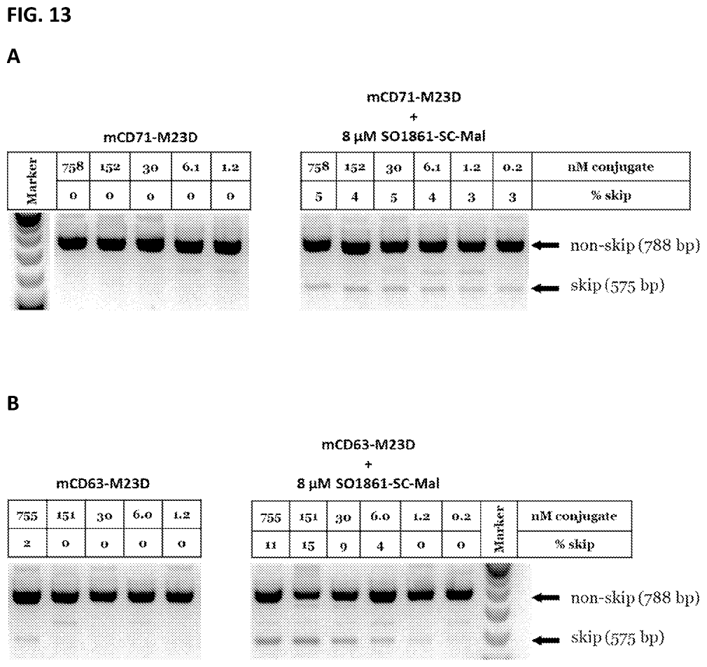

DBCO-(M23D)₂, a branched scaffold bearing two M23D (a phosphorodiamidate morpholino oligomer antisense oligonucleotide that induces exon 23 skipping of mouse dystrophin) oligonucleotide payloads, was produced as shown in FIG. 11. DBCO-(M23D)₂ was conjugated to either anti-CD71 monoclonal antibody targeting murine CD71 or anti-CD63 monoclonal antibody targeting murine CD63, to produce mCD71-M23D (DAR 3.5) and mCD63-M23D (DAR 3.4), respectively (for conjugation procedure see FIG. 12). Either mCD71-M23D conjugate or mCD63-M23D conjugate was co-administered with a fixed concentration of 8 μM of the endosomal escape enhancer SO1861-SC-Mal and tested for dystrophin exon 23 skipping on differentiated C2C12 murine myotubes following 48 h of treatment. Co-administration of 8 μM SO1861-SC-Mal revealed strong exon 23 skipping enhancement for both mCD71-M23D (clear band until 0.2 nM, an improvement of at least three orders of magnitude) (FIG. 13A, right panel) and mCD63-M23D (clear band until 6.0 nM, an improvement of two orders of magnitude) (FIG. 13B, right panel). mCD71-M23D alone showed no activity at all concentrations tested up to 758 nM (FIG. 13A, left panel), and mCD63-M23D alone showed only 2% skip at 755 nM (FIG. 13B, left panel). Treatments did not affect cell viability, as determined with a CTG assay. These data show that co-administration of SO1861-SC-Mal improves CD71- and CD63-targeted delivery of M23D in myotubes by at least two to three orders of magnitude.

### Example 6. hCD71-5′-SS-DMD-ASO+SO1861-SC-Mal or hCD71-5′-SS-DMD-PMO(1)+SO1861-SC-Mal or hCD71-3′-SS-DMD-PMO(1, 2, 3, 4, or 5)+SO1861-SC-Mal (In Vitro)

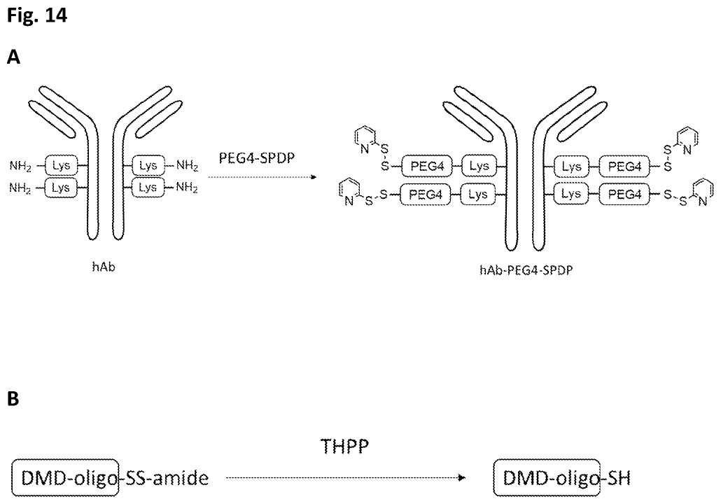

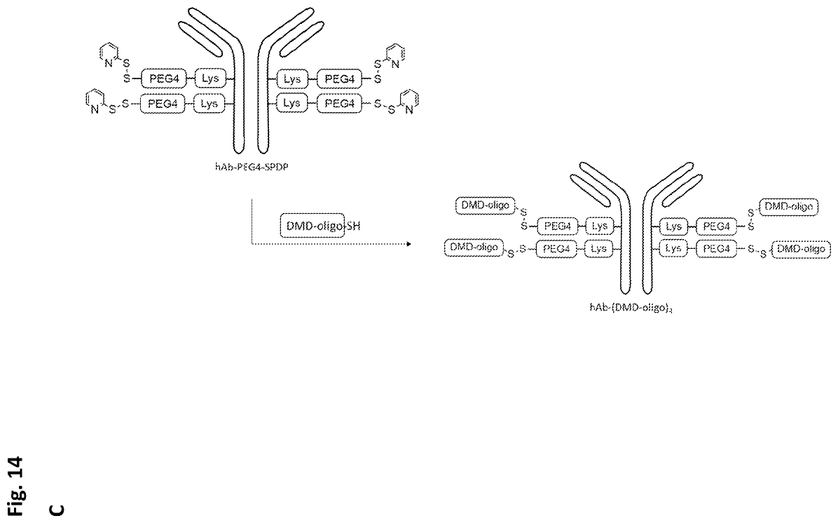

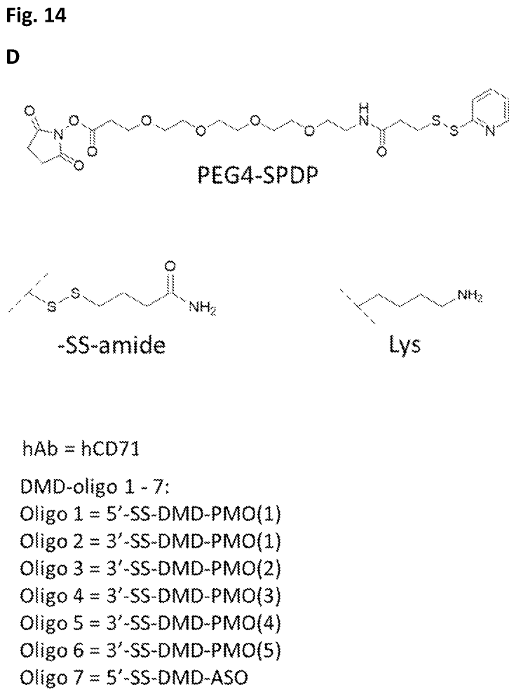

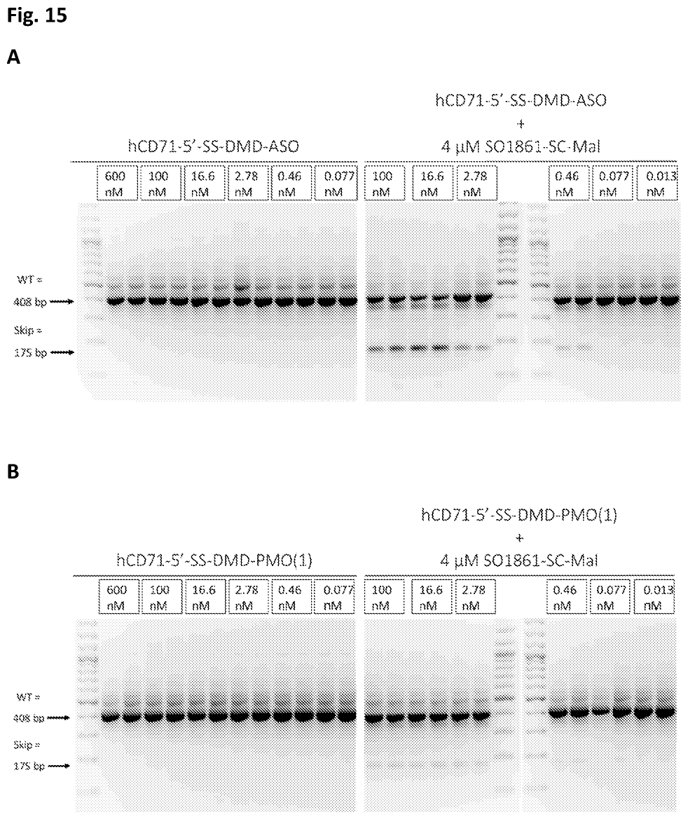

To anti-CD71 monoclonal antibody targeting human CD71, DMD-ASO-SH (activated form of a 2′O-methyl-phosporothioate antisense oligonucleotide that induces exon 51 skipping of human dystrophin and has the same sequence and chemistry modifications as drisapersen) or DMD-PMO(1)-SH (activated form of a phosphorodiamidate morpholino oligomer [PMO] antisense oligonucleotide that induces exon 51 skipping of human dystrophin and has the same sequence but not 5′-modifications as eteplirsen) was conjugated through disulfide bond formation on the 5′ to produce hCD71-5′-SS-DMD-ASO (DAR2.1) and hCD71-5′-SS-DMD-PMO(1) (DAR2.2), respectively (for conjugation procedure see FIG. 14A-D). The resultant compounds were tested for dystrophin exon 51 skipping, either without or in combination with 4 μM of the endosomal escape enhancer SO1861-SC-Mal, on differentiated human myotubes from a non-DMD donor (KM155). This revealed strongly enhanced skipping of exon 51 in combination with 4 μM SO1861-SC-Mal after 72 hrs of treatment: while hCD71-5′-SS-DMD-ASO alone resulted in low exon 51 skipping (2.6%) at 600 nM (FIG. 15A, left panel; Table A8), already 0.46-100 nM hCD71-5′-SS-DMD-ASO+SO1861-SC-Mal revealed 13.4-43.6% exon 51 skipping (FIG. 15A, right panel; Table A9). Likewise, while exposure to hCD71-5′-SS-DMD-PMO(1) resulted in very minor exon 51 skipping at 16.6-600 nM (0.4-1.1%) (FIG. 15B, left panel; Table A8), exon 51 skipping increased to 10.4-12.5% at a 6-fold lower exposure concentration of 2.78-100 nM hCD71-5′-SS-DMD-PMO(1)+SO1861-SC-Mal (FIG. 15B, right panel; Table A9), constituting a marked improvement in potency compared to conditions without SO1861-SC-Mal. Thus, co-administration of SO1861-SC-Mal with hCD71-5′-SS-DMD-ASO and hCD71-5′-SS-DMD-PMO(1), i.e. targeted DMD oligonucleotides that are conjugated to hCD71 on the 5′, improves on-target delivery and induces marked exon 51 skipping.

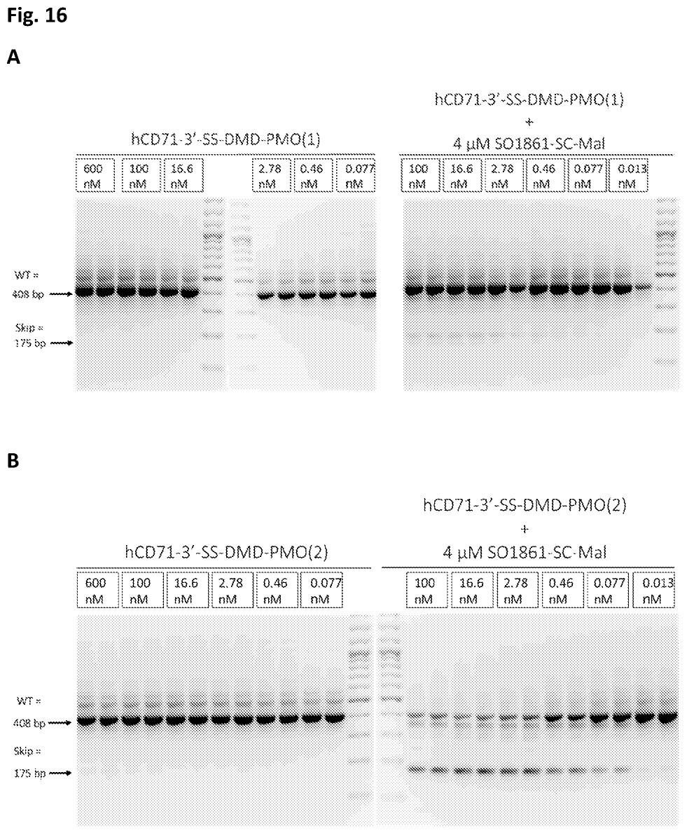

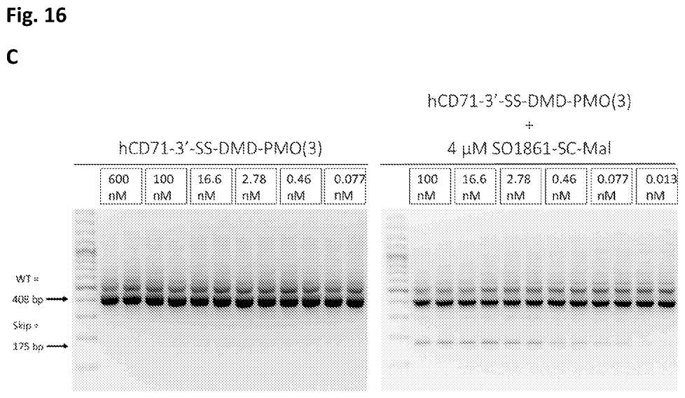

Next, targeted DMD-PMOs that are conjugated to anti-hCD71 on the 3′ were tested for exon 51 skipping activity on human myotubes. To anti-CD71 monoclonal antibody targeting human CD71, DMD-PMO(1)-SH, DMD-PMO(2)-SH (activated form of a PMO antisense oligonucleotide that induces exon 51 skipping of human dystrophin, as described in Echigoya et al. (2017)) or DMD-PMO(3)-SH (activated form of a PMO antisense oligonucleotide that induces exon 51 skipping of human dystrophin, as described in Echigoya et al. (2017)) was conjugated through disulfide bond formation on the 3′ to produce hCD71-3′-SS-DMD-PMO(1) (DAR2.1), hCD71-3′-SS-DMD-PMO(2) (DAR3.0), and hCD71-3′-SS-DMD-PMO(3) (DAR2.6), respectively (for conjugation procedure see FIG. 14A-D). The resultant compounds were tested for dystrophin exon 51 skipping, either without or in combination with 4 μM SO1861-SC-Mal, on differentiated human myotubes from a non-DMD donor (KM155). As shown for hCD71-5′-SS-DMD-PMO(1), this revealed enhanced exon 51 skipping for hCD71-3′-SS-DMD-PMO(1) in combination with 4 μM SO1861-SC-Mal after 72 hrs of treatment: while exposure to hCD71-3′-SS-DMD-PMO(1) alone resulted in very minor exon 51 skipping at 0.077-600 nM (0.4-2.2%) (FIG. 16A, left panel; Table A8), exon 51 skipping increased to 8.2-9.7% at an exposure concentration of 2.78-100 nM hCD71-3′-SS-DMD-PMO(1)+SO1861-SC-Mal (FIG. 16A, right panel; Table A9). More strikingly, exon 51 skipping was strongly enhanced for hCD71-3′-SS-DMD-PMO(2) in combination with 4 μM SO1861-SC-Mal: exposure to hCD71-3′-SS-DMD-PMO(2)+SO1861-SC-Mal revealed already exon 51 skipping (7.8%) at 0.013 nM conjugate, which increased up to 75.6-80.4% at 2.78-100 nM conjugate (FIG. 16B, right panel; Table A9), while exposure to hCD71-3′-SS-DMD-PMO(2) alone resulted only in 5.7% exon 51 skipping at 600 nM conjugate (FIG. 16B, left panel; Table A8), constituting a four orders of magnitude improvement compared to conditions without SO1861-SC-Mal. Also exposure to hCD71-3′-SS-DMD-PMO(3) in combination with 4 μM SO1861-SC-Mal resulted in strongly enhanced exon 51 skipping: hCD71-3′-SS-DMD-PMO(3)+SO1861-SC-Mal revealed already exon 51 skipping (9.6%) at 0.077 nM conjugate, which increased up to 28.3-29.2% at 2.78-100 nM (FIG. 16C, right panel; Table A9), while exposure to hCD71-3′-SS-DMD-PMO(2) alone resulted in no exon 51 skipping (0.0%) at all concentrations tested (up to 600 nM) (FIG. 16C, left panel; Table A8). Thus, co-administration of SO1861-SC-Mal to hCD71-3′-SS-DMD-PMO(1), hCD71-3′-SS-DMD-PMO(2), and hCD71-3′-DMD-PMO(3), i.e. targeted DMD oligonucleotides that are conjugated to hCD71 on the 3′, improves on-target delivery and induces marked exon 51 skipping.

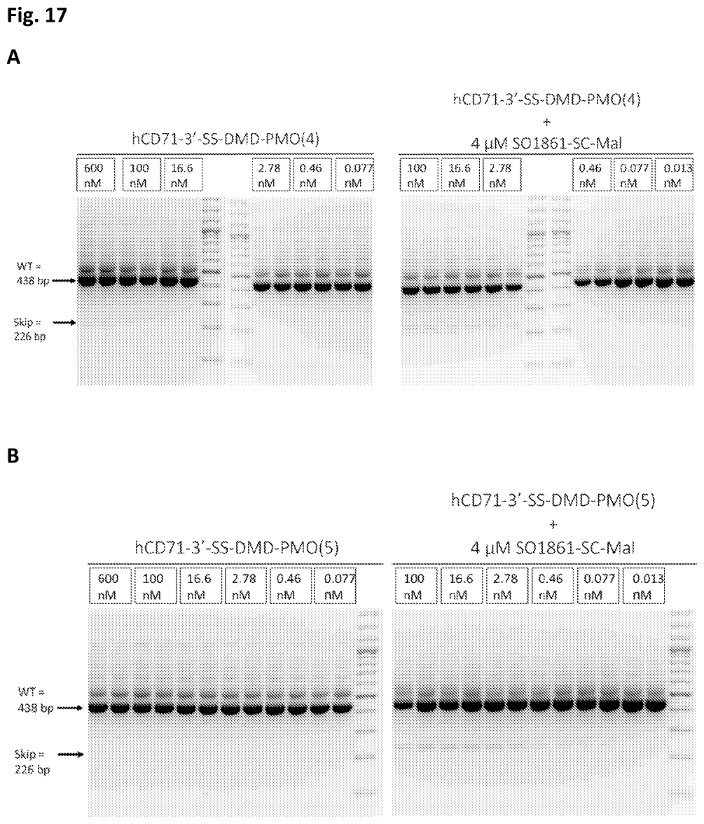

Also targeted DMD-PMOs that induce exon 53 skipping of human dystrophin were tested on human myotubes. To anti-CD71 monoclonal antibody targeting human CD71, DMD-PMO(4)-SH (activated form of a PMO antisense oligonucleotide that induces exon 53 skipping of human dystrophin and has the same sequence and chemistry modifications as golodirsen) or DMD-PMO(5)-SH (activated form of a PMO antisense oligonucleotide that induces exon 53 skipping of human dystrophin and has the same sequence and chemistry modifications as viltolarsen) was conjugated through disulfide bond formation on the 3′ to produce hCD71-3′-SS-DMD-PMO(4) (DAR2.3) and hCD71-3′-SS-DMD-PMO(5) (DAR2.1), respectively (for conjugation procedure see FIG. 14A-D). The resultant compounds were tested for dystrophin exon 53 skipping, either without or in combination with 4 μM SO1861-SC-Mal, on differentiated human myotubes from a non-DMD donor (KM155). This revealed enhanced exon 53 skipping only in combination with SO1861-SC-Mal after 72 hrs of treatment: while exposure to 600 nM hCD71-3′-SS-DMD-PMO(4) alone resulted in only 0.4% exon 53 skipping (FIG. 17A, left panel; Table A8), already 2.78 nM hCD71-3′-SS-DMD-PMO(4)+SO1861-SC-Mal resulted in 5.4% exon 53 skipping (FIG. 17A, right panel; Table A9). Likewise, while exposure to 600 nM hCD71-3′-SS-DMD-PMO(5) alone resulted in 0.3% exon 53 skipping (FIG. 17B, left panel; Table A8), already 2.78 nM hCD71-3′-SS-DMD-PMO(5)+SO1861-SC-Mal (FIG. 17B, right panel; Table A9) resulted in 6.0% exon 53 skipping, which increased up to 8.4% at 100 nM hCD71-3′-SS-DMD-PMO(5), realizing an improvement of one to two orders of magnitude. These data show that co-administration of SO1861-SC-Mal to hCD71-3′-SS-DMD-PMO(4) and hCD71-3′-SS-DMD-PMO(5), i.e. targeted DM0 oligonucleotides that are conjugated to hCD71 on the 3′, enhances their on-target delivery and induces exon 53 skipping.

Taken together, these data show that co-administration of SO1861-SC-Mal is broadly applicable to effectively increase the potency of hCD71-targeted DM0 oligonucleotides with different targeting sequences (different exons, different sequences) and conjugation methods (either on the 5′ or 3′ terminus) in human myotubes.

TABLE A8
Skip efficacy of anti-CD71-conjugate d DMD oligonucleotides in human myotubes

| % exon skipping | | [nM conjugate] | | | | | | |
|---|---|---|---|---|---|---|---|---|
| (Femto or Lab-on-a-chip analysis) | | 600 | 100 | 16.6 | 2.78 | 0.46 | 0.077 | |
| hCD71-5′-SS-DMD-ASO | Femto | 2.6 | 0.4 | 0.3 | 0.5 | 1.5 | 0.4 | % DMD |
| (DAR2.1) (KM155) | | | | | | | | exon 51 |
| hCD71-5′-SS-DMD-PMO(1) | Femto | 0.5 | 0.4 | 1.1 | 0.3 | 0.3 | 0.3 | skipping |
| (DAR2.2) (KM155) | | | | | | | | |
| hCD71-3′-SS-DMD-PMO(1) | Femto | 2.2 | 0.4 | 0.5 | 0.4 | 1.6 | 0.5 | |
| (DAR2.1) (KM155) | | | | | | | | |
| hCD71-3′-SS-DMD-PMO(2) | Femto | 5.7 | 1.3 | 2.6 | 0.3 | 0.4 | 0.5 | |
| (DAR3.0) (KM155) | | | | | | | | |
| hCD71-3′-SS-DMD-PMO(3) | Lab-on- | 0.0 | 0.0 | 0.0 | 0.0 | 0.0 | 0.0 | |
| (DAR2.6) (KM155) | a-chip | | | | | | | |
| hCD71-3′-SS-DMD-PMO(4) | Femto | 0.4 | 0.0 | 0.0 | 0.0 | 0.0 | 0.0 | % DMD |
| (DAR2.3) (KM155) | | | | | | | | exon 53 |
| hCD71-3′-SS-DMD-PMO(5) | Femto | 0.3 | 0.0 | 0.5 | 0.0 | 0.0 | 0.0 | skipping |
| (DAR2.1) (KM155) | | | | | | | | |

TABLE A9
Skip efficacy of anti-CD71-conjugated DMD oligonucleotides
with SO1861-SC-Mal co-administration in human myotubes

| % exon skipping | | [nM conjugate] | | | | | | |
|---|---|---|---|---|---|---|---|---|
| (Femto or Lab-on-a-chip analysis) | | 100 | 16.6 | 2.78 | 0.46 | 0.077 | 0.013 | |
| hCD71-5′-SS-DMD-ASO | Femto | 43.6 | 63.8 | 21.9 | 13.4 | 0.0 | 0.0 | % DMD |
| (DAR2.1) + SO1861-SC-Mal | | | | | | | | exon 51 |
| (KM155) | | | | | | | | skipping |
| hCD71-5′-SS-DMD-PMO(1) | Femto | 10.4 | 12.5 | 12.3 | 5.3 | 1.8 | 1.0 | |
| (DAR2.2) + SO1861-SC-Mal | | | | | | | | |
| (KM155) | | | | | | | | |
| hCD71-3′-SS-DMD-PMO(1) | Femto | 9.5 | 9.7 | 8.2 | 5.4 | 2.2 | 0.7 | |
| (DAR2.1) + SO1861-SC-Mal | | | | | | | | |
| (KM155) | | | | | | | | |
| hCD71-3′-SS-DMD-PMO(2) | Femto | 79.1 | 80.4 | 75.6 | 50.7 | 23.4 | 7.8 | |
| (DAR3.0) + SO1861-SC-Mal | | | | | | | | |
| (KM155) | | | | | | | | |
| hCD71-3′-SS-DMD-PMO(3) | Lab- | 29.2 | 29.1 | 28.3 | 14.6 | 9.6 | 1.0 | |
| (DAR2.6) + SO1861-SC-Mal | on-a- | | | | | | | |
| (KM155) | chip | | | | | | | |
| hCD71-3′-SS-DMD-PMO(4) | Femto | 4.4 | 5.0 | 5.4 | 1.3 | 0.6 | 0.1 | % DMD |
| (DAR2.3) + SO1861-SC-Mal | | | | | | | | exon 53 |
| (KM155) | | | | | | | | skipping |
| hCD71-3′-SS-DMD-PMO(5) + | Femto | 8.4 | 5.4 | 6.0 | 2.1 | 1.8 | 0.0 | |
| SO1861-SC-Mal (DAR2.1) | | | | | | | | |
| (KM155) | | | | | | | | |

### LITERATURE REFERENCES

Cardamone M, et al., 2008—Cardamone M; Darras B T; Ryan M M, 2008, ‘Inherited myopathies and muscular dystrophies’, Seminars in Neurology, vol. 28, pp. 250-259, http://dx.doi.org/10.1055/s-2008-1062269
Shadrin et al, 2016—Striated muscle function, regeneration, and repair, Cell Mol Life Sci. 2016 November; 73(22): 4175-4202. doi:10.1007/s00018-016-2285-z.
Dumont N A, Wang Y X, Rudnicki M A. Intrinsic and extrinsic mechanisms regulating satellite cell function. Development. 2015; 142(9):1572-1581, doi: 10.1242/dev.114223
Uygur and Lee, Mechanisms of Cardiac Regeneration. Dev Cell. 2016 Feb. 22; Volume 36(4): pp. 362-374, doi: 10.1016/j.devcel.2016.01.018
Slørdal L, Spigset O. Heart failure induced by non-cardiac drugs, Drug Saf 2006; Volume 29(7): pp. 567-86, doi: 10.2165/00002018-200629070-00003
Ebner et al., Strategies for Skeletal Muscle Targeting in Drug Discovery, Current Pharmaceutical Design 2015, Volume 21(10), pp. 1327-1336, DOI:10.2174/1381612820666140929095755
González-Jamett et al., Hereditary Myopathies, 2018, DOI: 10.5772/intechopen.76076, www.intechopen.com/chapters/60283
Savarese et al., Is Gene-Size an Issue for the Diagnosis of Skeletal Muscle Disorders?, 2020, Volume 7(3), pp. 203-216, DOI: 10.3233/JND-190459
Duan et al., Duchenne muscular dystrophy, Nat Rev Dis Primers, 2021 Feb. 18; Volume 7(1):13, doi: 10.1038/s41572-021-00248-3
Bladen, C. L. et al., The TREAT-NMD Duchenne muscular dystrophy registries: conception, design, and utilization by industry and academia. Hum. Mutat. 34, 1449-1457 (2013), DOI: 10.1002/humu.22390.
Goemans, N. et al., A randomized placebo-controlled phase 3 trial of an antisense oligonucleotide, drisapersen, in Duchenne muscular dystrophy. Neuromuscul. Disord. Volume 28(1), pp. 4-15 (2018), doi: 10.1016/j.nmd.2017.10.004.
Mendell, J. R. et al., Assessment of systemic delivery of rAAVrh74.MHCK7.micro-dystrophin in children with duchenne muscular dystrophy: a nonrandomized controlled trial. JAMA Neurol. Volume 77(9), pp. 1122-1131 (2020), doi: 10.1001/jamaneurol.2020.1484.
Mendell, J. R. et al, A phase I/IIa follistatin gene therapy trial for Becker muscular dystrophy, Mol. Ther. Volume 23(1), pp. 192-201 (2015), doi: 10.1038/mt.2014.200.
Wasala, N. B. et al., Single SERCA2a therapy ameliorated dilated cardiomyopathy for 18 months in a mouse model of Duchenne muscular dystrophy, Mol. Ther. Volume 28(3), pp. 845-854 (2020), doi: 10.1016/j.ymthe.2019.12.011
Arenega, et al., Review MiRNAs and Muscle Regeneration: Therapeutic Targets in Duchenne Muscular Dystrophy, Int J Mol Sci. (2021 April), Volume 22(8): 4236, doi: 10.3390/ijms22084236
Chemello, et al., Correction of muscular dystrophies by CRISPR gene editing, J. Clin. Invest. Volume 130(6), pp. 2766-2776 (2020), doi: 10.1172/JC1136873
Nelson, et al., Genome engineering: a new approach to gene therapy for neuromuscular disorders, Nat. Rev. Neurol. Volume 13(11), pp. 647-661 (2017), doi: 10.1038/nrneurol.2017.126
Cerro-Herreros et al., Therapeutic Potential of AntagomiR-23b for Treating Myotonic Dystrophy, Mol Ther Nucleic Acids, 2020 Sep. 4; Volume 21, pp. 837-849, doi.org/10.1016/j.omtn.2020.07.021
Bladen, C. L. et al., The TREAT-NMD DMD global database: analysis of more than 7000 Duchenne muscular dystrophy mutations. Hum. Mutat. Volume 36(4), pp. 395-402 (2015), doi: 10.1002/humu.22758
Hanahan and Weinberg, Hallmarks of cancer: the next generation, Cell (2011, Mar. 4); Volume 144(5), pp. 646-674, doi: 10.1016/j.cell.2011.02.013.
Wang et al., Saponins as Natural Adjuvant for Antisense Morpholino Oligonucleotides Delivery In Vitro and in mdx Mice, Molecular Therapy: Nucleic Acids, 2018, Volume 11, pp. 192-202, doi: 10.1016/j.omtn.2018.02.004
Wang et al., Saponins enhance exon skipping of 2′-O-methyl phosphorothioate oligonucleotide in vitro and in vivo, Drug Design, Development and Therapy Volume 12: pp. 3705-3715 (2018), DOI:10.2147/DDDT.S179008
Kemaladewi et al, Dual exon skipping in myostatin and dystrophin for Duchenne muscular dystrophy, BMC Medical Genomics, Volume 4, 36 (2011), DOI: 10.1186/1755-8794-4-36
Baik et al, Cell type-selective targeted delivery of a recombinant lysosomal enzyme for enzyme therapies, Molecular Therapy, Volume 29(12), pp. 3512-3524 (2021), DOI: 10.1016/j.ymthe.2021.08.020
Echigoya et al., Quantitative antisense screening and optimization for exon 51 skipping in Duchenne muscular dystrophy, Molecular Therapy, Volume 25(11), pp. 2561-2572 (2017), DOI: 10.1016/j.ymthe.2017.07.014

1. A pharmaceutical composition for use in the treatment or prophylaxis of a muscle wasting disorder, the composition comprising
a nucleic acid, and
a saponin,
wherein the saponin is a triterpenoid 12,13-dehydrooleanane-type saponin comprising an aldehyde group at position C-23 of the saponin's aglycone core structure under acidic conditions present in endosomes and/or lysosomes of human cells.

2. Composition for use according to claim 1, wherein the muscle wasting disorder is a muscle cell-related genetic disorder, preferably being a congenital myopathy or a muscular dystrophy;
preferably wherein the congenital myopathy is selected from nemaline myopathy or congenital fiber-type disproportion myopathy, and/or wherein the muscular dystrophy is selected from a dystrophinopathy, facioscapulohumeral muscular dystrophy, myotonic dystrophy, Emery-Dreifuss muscular dystrophy, limb-girdle muscular dystrophy 1B, congenital muscular dystrophy; or dilated familial cardiomyopathy;
most preferably wherein the muscle wasting disorder is a muscle cell-related genetic disorder being a dystrophinopathy, preferably being Duchenne muscular dystrophy.

3. Composition for use according to claim 1 or 2, wherein the treatment or prophylaxis of the muscle wasting disorder involves antisense therapy, preferably involving exon skipping.

4. Composition for use according to any one of the preceding claims, wherein the aldehyde group at position C-23 of the saponin's aglycone core structure is either a free aldehyde group, or is an aldehyde group substituted by a maleimide-comprising moiety attached at said position C-23 with a cleavable covalent bond that cleaves off under acidic conditions present in endosomes and/or lysosomes of human cells, wherein said aldehyde group at position C-23 of the saponin's aglycone core structure is restored upon said cleavage under acidic conditions present in endosomes and/or lysosomes of human cells;
preferably wherein the cleavable covalent bond is selected from a semicarbazone bond, a hydrazone bond, or a hydrazide bond; more preferably being selected from a semicarbazone bond and a hydrazone bond; most preferably being a hydrazone bond.

5. Composition for use according to claim 4, wherein the maleimide-comprising moiety is part of a molecule comprising or consisting of 4-(6-(2,5-dioxo-2,5-dihydro-1H-pyrrol-1-yl)hexanoyl)piperazine-1-carbohydrazide that is attached at position C-23 of the saponin's aglycone core structure upon forming a semicarbazone bond (further referred to as SC-Maleimide) or wherein the maleimide-comprising moiety is part of a molecule comprising or consisting of N-ε-maleimidocaproic acid hydrazide that is attached at position C-23 of the saponin's aglycone core structure upon forming a hydrazone bond (further referred to as EMCH).

6. Composition for use according to any one of the preceding claims, wherein the saponin is a free saponin defined as consisting of one or more saponin molecules devoid of a direct or indirect covalent conjugation to a macromolecule such as a nucleic acid or a ligand.

7. Composition for use according to any one of the preceding claims, wherein the saponin's aglycone core structure is selected from any one or more of quillaic acid, gypsogenin, and a derivative thereof, preferably wherein the saponin's aglycone core structure is quillaic acid or gypsogenin, more preferably quillaic acid.

8. Composition for use according to any one of the preceding claims, wherein the saponin is at least a bidesmosidic saponin comprising a first saccharide chain that comprises a terminal glucuronic acid residue and comprises a second saccharide chain that comprises at least four sugar residues in a branched configuration;
preferably wherein the first saccharide chain is Gal-(1→2)-[Xyl-(1→3)]-GlcA and/or wherein the branched second saccharide chain of at least four sugar residues comprises a terminal fucose residue and/or a terminal rhamnose residue.

9. Composition for use according to claim 8, wherein the saponin comprises the first saccharide chain at position C-3 of the saponin's aglycone core structure and/or the second saccharide chain at position C-28 of the saponin's aglycone core structure;
preferably wherein the first saccharide chain is a carbohydrate substituent at the C-3beta-OH group of the saponin's aglycone core structure and/or wherein the second saccharide chain is a carbohydrate substituent at the C-28-OH group of the saponin's aglycone core structure.

10. Composition for use according to any one of the preceding claims, wherein the saponin is any one or more of:
a) saponin selected from any one or more of list A:
Quillaja saponaria saponin mixture, or a saponin isolated from Quillaja saponaria, for example Quil-A, QS-17-api, QS-17-xyl, QS-21, QS-21A, QS-21B, QS-7-xyl;
Saponinum album saponin mixture, or a saponin isolated from Saponinum album;
Saponaria officinalis saponin mixture, or a saponin isolated from Saponaria officinalis; and
Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B:
SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C:
AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS-7, QS-7 api, QS-17, QS-18, QS-21 A-apio, QS-21 A-xylo, QS-21 B-apio and QS-21 B-xylo; or
preferably, the saponin is any one or more of a saponin selected from list B or C, more preferably, a saponin selected from list C.

11. Composition for use according to any one of the preceding claims, wherein the saponin is any one or more of AG1856, GE1741, a saponin isolated from Quillaja saponaria, Quil-A, QS-17, QS-21, QS-7, SA1641, a saponin isolated from Saponaria officinalis, Saponarioside B, SO1542, SO1584, SO1658, SO1674, SO1700, SO1730, SO1772, SO1832, SO1861, SO1862 and SO1904;
preferably wherein the saponin is any one or more of QS-21, SO1832, AG1856, SO1861, SA1641 and GE1741; more preferably wherein the saponin is QS-21, SO1832 or SO1861; most preferably being SO1861.

12. Composition for use according to any one of the preceding claims, wherein the saponin is a saponin isolated from Saponaria officinalis, preferably wherein the saponin is any one or more of Saponarioside B, SO1542, SO1584, SO1658, SO1674, SO1700, SO1730, SO1772, SO1832, SO1861, SO1862 and SO1904;
more preferably wherein the saponin is any one or more of SO1832, SO1861 and SO1862;
even more preferably wherein the saponin is SO1832 and SO1861;
most preferably being SO1861.

13. Composition for use according to any one of the preceding claims, wherein the nucleic acid is an oligonucleotide defined as a nucleic acid that is no longer than 150 nt, preferably wherein the oligonucleotide has a size of 5-150 nt, preferably being 8-100 nt, most preferably being 10-50 nt, more preferably wherein the oligonucleotide is an antisense oligonucleotide, even more preferably being a mutation specific antisense oligonucleotide, most preferably being an oligonucleotide designed to induce exon skipping.

14. Composition for use according to any one of the preceding claims, wherein the nucleic acid is a plasmid or another circular genetic construct.

15. Composition for use according to any one of the preceding claims, wherein the nucleic acid comprises or consists of any one of the following: morpholino phosphorodiamidate oligomer (PMO), 2′-O-methyl (2′-OMe) phosphorothioate RNA, 2′-O-methoxyethyl (2′-O-MOE) RNA {2′-O-methoxyethyl-RNA (MOE)}, locked or bridged nucleic acid (BNA), 2′-0,4′-aminoethylene bridged nucleic acid (BNANC), peptide nucleic acid (PNA), 2′-deoxy-2′-fluoroarabino nucleic acid (FANA), 3′-fluoro hexitol nucleic acid (FHNA), glycol nucleic acid (GNA), threose nucleic acid (TNA), silencing RNA (siRNA), short hairpin RNA (shRNA), microRNA (miRNA), antagomir (miRNA antagonists), aptamer RNA or aptamer DNA, single-stranded RNA or single-stranded DNA, double-stranded RNA (dsRNA) or double-stranded DNA;
preferably wherein the nucleic acid comprises or consists of a morpholino phosphorodiamidate oligomer (PMO) or a 2′-O-methyl (2′-OMe) phosphorothioate RNA.

16. Composition for use according to any one of the preceding claims, wherein the nucleic acid is designed to induce exon skipping of the human dystrophin gene transcript, preferably wherein the exon skipping involves exon 51 skipping or exon 53 skipping or exon 45 skipping;
more preferably wherein the nucleic acid is a 2′O-methyl-phosporothioate antisense oligonucleotide or a phosphorodiamidate morpholino oligomer antisense oligonucleotide that is designed to induce the exon 51 skipping or the exon 53 skipping or the exon 45 skipping,
even more preferably wherein the nucleic acid is selected from eteplirsen, drisapersen, golodirsen, viltolarsen, and casimersen.

17. Composition for use according to any one of the preceding claims, wherein the composition comprises two or more different nucleic acids, preferably being two or more different oligonucleotides, more preferably wherein at least one of the two or more different oligonucleotides is an antisense oligonucleotide.

18. Composition for use according to any one of the preceding claims, wherein the nucleic acid is conjugated with a ligand of an endocytic receptor on a muscle cell.

19. Composition for use according to claim 18, wherein the endocytic receptor on a muscle cell to which the ligand binds is selected from: transferrin receptor (CD71), insulin-like growth factor 1 (IGF-1) receptor (IGF1R), tetraspanin CD63; muscle-specific kinase (MuSK), glucose transporter GLUT4, cation independent mannose 6 phosphate receptor (CI-MPR).

20. Composition for use according to claim 18-19, wherein the ligand is selected from any one of:
insulin-like growth factor 1 (IGF-I) or a fragment thereof;
insulin-like growth factor 2 (IGF-II) or a fragment thereof
Mannose 6 phosphate
transferrin (Tf),
zymozan A, and
an antibody or a binding fragment thereof specific for binding to the endocytic receptor, wherein the endocytic receptor is preferably selected from: transferrin receptor (CD71), insulin-like growth factor 1 (IGF-I) receptor (IGF-IR), tetraspanin CD63, muscle-specific kinase (MuSK), glucose transporter GLUT4, cation independent mannose 6 phosphate receptor (CI-MPR), and LDL receptor;
preferably wherein the ligand is an antibody or a binding fragment thereof that is specific for binding to a transferrin receptor,
more preferably wherein the ligand is a monoclonal antibody or a Fab′ fragment or at least one single domain antibody specific for binding to a transferrin receptor, even more preferably wherein the ligand is a monoclonal antibody specific for binding to a transferrin receptor.

21. Composition for use according to any one of the claims 18-20, wherein the ligand is conjugated with 2-5 molecules of the nucleic acid per 1 molecule of the ligand;
preferably being 3-4 molecules of the nucleic acid per 1 molecule of the ligand;
more preferably wherein the ligand is on average conjugated with 4 molecules of the nucleic acid per 1 molecule of the ligand.

22. Composition for use according to any one of the claims 18-21, wherein the ligand comprises a chain of amino acid residues comprising at least one cysteine residue and/or at least one lysine residue and wherein the covalent linking of the nucleic acid with the ligand comprises a covalent bond with at least one cysteine residue and/or at least one lysine residue;
preferably wherein more than one molecule of the nucleic acid is linked to one molecule of the ligand via a separate cysteine residue and/or a separate lysine residue;
more preferably wherein the ligand comprises a chain of amino acid residues comprising a multicysteine repeat, possibly being a tetracysteine repeat represented by the sequence HRWCCPGCCKTF (SEQ ID NO.4), and wherein the covalent linking of the nucleic acid with the ligand comprises a covalent bond with any one or more of the cysteine residues of the multicysteine repeat;
most preferably wherein more than one molecule of the nucleic acid is linked to one molecule of the ligand via a separate cysteine residue of the multicysteine repeat.

23. Composition for use according to any one of the claims 18-22, wherein the covalent linking of the nucleic acid with the ligand is made via a linker to which the nucleic acid is covalently bound;
preferably wherein the linker comprises or consists of linker succinimidyl 3-(2-pyridyldithio)propionate (SPDP); possibly wherein the linker covalently links the nucleic acid to a lysine residue of the ligand, or to a glycan residue, preferably a partially-trimmed glycan.

24. Composition for use according to claim 23, wherein the linker is a cleavable linker subject to cleavage under acidic, reductive, enzymatic and/or light-induced conditions;
preferably wherein the linker comprises a cleavable bond selected from:
a bond subject to cleavage under acidic conditions such as a semicarbazone bond, a hydrazone bond, an imine bond, an acetal bond including a 1,3-dioxolane bond, a ketal bond, an ester bond, and/or an oxime bond;
a bond susceptible to proteolysis, for example amide or peptide bond, preferably subject to proteolysis by Cathepsin B;
a red/ox-cleavable bond such as a disulfide bond, or a thiol-exchange reaction-susceptible bond such as a thio-ether bond;
preferably being an acid-sensitive bond subject to cleavage in vivo under acidic conditions present in endosomes and/or lysosomes of human cells, preferably of pH≤6.5, preferably pH≤6, more preferably pH≤5.5,
more preferably being an acid-sensitive bond selected from a semicarbazone bond and a hydrazone bond; most preferably being a hydrazone bond.

25. Composition for use according to any one of the preceding claims, wherein the saponin is or comprises at least one molecule of any of SO1861, SO1861-EMCH, or SO1861-SC-Maleimide, preferably SO1861-EMCH or SO1861-SC-Maleimide, the nucleic acid is drisapersen or eteplirsen, and the ligand conjugated with the nucleic acid is antiCD71 antibody or a binding fragment thereof.

26. Composition for use according to any one of the preceding claims, for use in intravenous or subcutaneous administration to a human subject.

27. Composition for use according to any one of the preceding claims, the composition comprising a pharmaceutically acceptable excipient and/or pharmaceutically acceptable diluent.

28. Composition for use according to any one of the preceding claims, the composition comprising 2-6 μM of the saponin, preferably being 3-5 μM, more preferably being 3.5-4.5 μM, and most being about 4 μM.

29. Therapeutic combination,
the therapeutic combination comprising:
(a) nucleic acid, and
(b) a saponin
wherein the saponin is a triterpenoid 12,13-dehydrooleanane-type saponin comprising an aldehyde group at position C-23 of the saponin's aglycone core structure under acidic conditions present in endosomes and/or lysosomes of human cells.

30. Therapeutic combination according to claim 29, wherein the aldehyde group at position C-23 of the saponin's aglycone core structure is either a free aldehyde group, or is an aldehyde group substituted by a maleimide-comprising moiety attached at said position C-23 with a cleavable covalent bond that cleaves off under acidic conditions present in endosomes and/or lysosomes of human cells, wherein said aldehyde group at position C-23 of the saponin's aglycone core structure is restored upon said cleavage under acidic conditions present in endosomes and/or lysosomes of human cells;
preferably wherein the cleavable covalent bond is selected from a semicarbazone bond, a hydrazone bond, or a hydrazide bond; more preferably being selected from a semicarbazone bond and a hydrazone bond; most preferably being a hydrazone bond.

31. Therapeutic combination according to claim 30, wherein the maleimide-comprising moiety is part of a molecule comprising or consisting of 4-(6-(2,5-dioxo-2,5-dihydro-1H-pyrrol-1-yl)hexanoyl)piperazine-1-carbohydrazide that is attached at position C-23 of the saponin's aglycone core structure upon forming a semicarbazone bond (further referred to as SC-Maleimide) or wherein the maleimide-comprising moiety is part of a molecule comprising or consisting of N-ε-maleimidocaproic acid hydrazide that is attached at position C-23 of the saponin's aglycone core structure upon forming a hydrazone bond (further referred to as EMCH).

32. Therapeutic combination according to any one of the claims 29-31, wherein the saponin is a free saponin defined as consisting of one or more saponin molecules devoid of a direct or indirect covalent conjugation to a macromolecule such as a nucleic acid or a ligand.

33. Therapeutic combination according to any one of the claims 29-32, wherein the saponin's aglycone core structure is selected from any one or more of quillaic acid, gypsogenin, and a derivative thereof, preferably wherein the saponin's aglycone core structure is quillaic acid or gypsogenin, more preferably quillaic acid.

34. Therapeutic combination according to any one of the claims 29-33, wherein the saponin is at least a bidesmosidic saponin comprising a first saccharide chain that comprises a terminal glucuronic acid residue and comprises a second saccharide chain that comprises at least four sugar residues in a branched configuration;
preferably wherein the first saccharide chain is Gal-(1→2)-[Xyl-(1→3)]-GlcA and/or wherein the branched second saccharide chain of at least four sugar residues comprises a terminal fucose residue and/or a terminal rhamnose residue.

35. Therapeutic combination according to claim 34, wherein the saponin comprises the first saccharide chain at position C-3 of the saponin's aglycone core structure and/or the second saccharide chain at position C-28 of the saponin's aglycone core structure;
preferably wherein the first saccharide chain is a carbohydrate substituent at the C-3beta-OH group of the saponin's aglycone core structure and/or wherein the second saccharide chain is a carbohydrate substituent at the C-28-OH group of the saponin's aglycone core structure.

36. Therapeutic combination according to any one of the claims 29-35, wherein the saponin is any one or more of:
a) saponin selected from any one or more of list A:
Quillaja saponaria saponin mixture, or a saponin isolated from Quillaja saponaria, for example Quil-A, QS-17-api, QS-17-xyl, QS-21, QS-21A, QS-21B, QS-7-xyl;
Saponinum album saponin mixture, or a saponin isolated from Saponinum album;
Saponaria officinalis saponin mixture, or a saponin isolated from Saponaria officinalis; and
Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B:
SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C:
AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS-7, QS-7 api, QS-17, QS-18, QS-21 A-apio, QS-21 A-xylo, QS-21 B-apio and QS-21 B-xylo; or
preferably, the saponin is any one or more of a saponin selected from list B or C, more preferably, a saponin selected from list C.

37. Therapeutic combination according to any one of the claims 29-36, wherein the saponin is any one or more of AG1856, GE1741, a saponin isolated from Quillaja saponaria, Quil-A, QS-17, QS-21, QS-7, SA1641, a saponin isolated from Saponaria officinalis, Saponarioside B, SO1542, SO1584, SO1658, SO1674, SO1700, SO1730, SO1772, SO1832, SO1861, SO1862 and SO1904;
preferably wherein the saponin is any one or more of QS-21, SO1832, AG1856, SO1861, SA1641 and GE1741; more preferably wherein the saponin is QS-21, SO1832 or SO1861;
most preferably being SO1861.

38. Therapeutic combination according to any one of the claims 29-37, wherein the saponin is a saponin isolated from Saponaria officinalis, preferably wherein the saponin is any one or more of Saponarioside B, SO1542, SO1584, SO1658, SO1674, SO1700, SO1730, SO1772, SO1832, SO1861, SO1862 and SO1904;
more preferably wherein the saponin is any one or more of SO1832, SO1861 and SO1862;
even more preferably wherein the saponin is SO1832 and SO1861;
most preferably being SO1861.

39. Therapeutic combination according to any one of the claims 29-38, wherein the nucleic acid is an oligonucleotide defined as a nucleic acid that is no longer than 150 nt, preferably wherein the oligonucleotide has a size of 5-150 nt, preferably being 8-100 nt, most preferably being 10-50 nt, more preferably wherein the oligonucleotide is an antisense oligonucleotide, preferably being a mutation specific antisense oligonucleotide, most preferably being an oligonucleotide designed to induce exon skipping.

40. Therapeutic combination according to any one of the claims 29-39, wherein the nucleic acid is a plasmid or another circular genetic construct.

41. Composition for use according to claim 29-40, wherein the nucleic acid comprises or consists of any one of the following: morpholino phosphorodiamidate oligomer (PMO), 2′-O-methyl (2′-OMe) phosphorothioate RNA, 2′-O-methoxyethyl (2′-O-MOE) RNA {2′-O-methoxyethyl-RNA (MOE)}, locked or bridged nucleic acid (LNA or BNA), 2′-O,4′-aminoethylene bridged nucleic acid (BNANC), peptide nucleic acid (PNA), 2′-deoxy-2′-fluoroarabino nucleic acid (FANA), 3′-fluoro hexitol nucleic acid (FHNA), glycol nucleic acid (GNA), threose nucleic acid (TNA), silencing RNA (siRNA), short hairpin RNA (shRNA), microRNA (miRNA), antagomir (miRNA antagonists), aptamer RNA or aptamer DNA, single-stranded RNA or single-stranded DNA, double-stranded RNA (dsRNA) or double-stranded DNA;
preferably wherein the nucleic acid comprises or consists of a morpholino phosphorodiamidate oligomer (PMO) or a 2′-O-methyl (2′-OMe) phosphorothioate RNA.

42. Therapeutic combination according to any one of the claims 39-41, wherein the oligonucleotide is designed to induce exon skipping of the human dystrophin gene transcript, preferably wherein the exon skipping involves exon 51 skipping or exon 53 skipping or exon 45 skipping;
more preferably wherein the oligonucleotide is a 2′O-methyl-phosporothioate antisense oligonucleotide or a phosphorodiamidate morpholino oligomer antisense oligonucleotide that is designed to induce the exon 51 skipping or the exon 53 skipping or the exon 45 skipping,
even more preferably wherein the oligonucleotide is selected from eteplirsen, drisapersen, golodirsen, viltolarsen, and casimersen.

43. Therapeutic combination according to any one of the claims 29-42, wherein the composition comprises two or more different nucleic acids, the two or more different nucleic acids preferably being two or more different oligonucleotides, more preferably wherein at least one of the two or more different oligonucleotides is an antisense oligonucleotide.

44. Therapeutic combination according to any one of the claims 29-43, wherein the nucleic acid is conjugated with a ligand of an endocytic receptor on a muscle cell.

45. Therapeutic combination according to claim 44, wherein the endocytic receptor on a muscle cell to which the ligand binds is selected from: transferrin receptor (CD71), insulin-like growth factor 1 (IGF-I) receptor (IGF-IR), tetraspanin CD63; muscle-specific kinase (MuSK), glucose transporter GLUT4, cation independent mannose 6 phosphate receptor (CI-MPR).

46. Therapeutic combination according to claim 44 or 45, wherein the ligand is selected from any one of:
insulin-like growth factor 1 (IGF-I) or a fragment thereof;
insulin-like growth factor 2 (IGF-II) or a fragment thereof
Mannose 6 phosphate
transferrin (Tf),
zymozan A, and
an antibody or a binding fragment thereof specific for binding to the endocytic receptor, wherein
the endocytic receptor is preferably selected from: transferrin receptor (CD71), insulin-like growth factor 1 (IGF-I) receptor (IGF-IR), tetraspanin CD63, muscle-specific kinase (MuSK), glucose transporter GLUT4, cation independent mannose 6 phosphate receptor (CI-MPR), and LDL receptor;
preferably wherein the ligand is an antibody or a binding fragment thereof that is specific for binding to a transferrin receptor,
more preferably wherein the ligand is a monoclonal antibody or a Fab′ fragment or at least one single domain antibody specific for binding to a transferrin receptor, even more preferably wherein the ligand is a monoclonal antibody specific for binding to a transferrin receptor.

47. Therapeutic combination according to any one of the claims 44-46, wherein the ligand is conjugated with 2-5 molecules of the nucleic acid per 1 molecule of the ligand;
preferably being 3-4 molecules of the nucleic acid per 1 molecule of the ligand;
more preferably wherein the ligand is on average conjugated with 4 molecules of the nucleic acid per 1 molecule of the ligand.

48. Therapeutic combination according to any one of the claims 44-47, wherein the ligand comprises a chain of amino acid residues comprising at least one cysteine residue and/or at least one lysine residue and wherein the covalent linking of the nucleic acid with the ligand comprises a covalent bond with at least one cysteine residue and/or at least one lysine residue;
preferably wherein more than one molecule of the nucleic acid is linked to one molecule of the ligand via a separate cysteine residue and/or a separate lysine residue;
more preferably wherein the ligand comprises a chain of amino acid residues comprising a multicysteine repeat, possibly being a tetracysteine repeat represented by the sequence HRWCCPGCCKTF (SEQ ID NO.4), and wherein the covalent linking of the nucleic acid with the ligand comprises a covalent bond with any one or more of the cysteine residues of the multicysteine repeat;
most preferably wherein more than one molecule of the nucleic acid is linked to one molecule of the ligand via a separate cysteine residue of the multicysteine repeat.

49. Therapeutic combination according to any one of the claims 44-48, wherein the covalent linking of the nucleic acid with the ligand is made via a linker to which the nucleic acid is covalently bound;
preferably wherein the linker comprises or consists of linker succinimidyl 3-(2-pyridyldithio)propionate (SPDP); possibly wherein the linker covalently links the nucleic acid to a lysine residue of the ligand, or to a glycan residue, preferably a partially-trimmed glycan.

50. Therapeutic combination according to claim 49 wherein the linker is a cleavable linker subject to cleavage under acidic, reductive, enzymatic and/or light-induced conditions;
preferably wherein the linker comprises a cleavable bond selected from:
a bond subject to cleavage under acidic conditions such as a semicarbazone bond, a hydrazone bond, an imine bond, an acetal bond including a 1,3-dioxolane bond, a ketal bond, an ester bond, and/or an oxime bond,
a bond susceptible to proteolysis, for example amide or peptide bond, preferably subject to proteolysis by Cathepsin B;
a red/ox-cleavable bond such as a disulfide bond, or a thiol-exchange reaction-susceptible bond such as a thio-ether bond
preferably being an acid-sensitive bond subject to cleavage in vivo under acidic conditions present in endosomes and/or lysosomes of human cells, preferably of pH≤6.5, preferably pH≤6, more preferably pH≤5.5,
more preferably being an acid-sensitive bond selected from a semicarbazone bond and a hydrazone bond; most preferably being a hydrazone bond.

51. Therapeutic combination according to any one of the claims 29-50, wherein the saponin is or comprises at least one molecule of any of SO1861, SO1861-EMCH or SO1861-SC-Maleimide, preferably SO1861-EMCH or SO1861-SC-Maleimide, the nucleic acid is drisapersen or eteplirsen, and the ligand conjugated with the nucleic acid is antiCD71 antibody or a binding fragment thereof.

52. Therapeutic combination according to any one of the claims 29-51, comprising a pharmaceutically acceptable excipient and/or pharmaceutically acceptable diluent.

53. Therapeutic combination according to any one of the claims 29-52, comprising 2-6 μM of the saponin, preferably being 3-5 μM, more preferably being 3.5-4.5 μM, and most being about 4 μM.

54. Kit comprising the components (a) and (b) of the therapeutic combination according to any one of the claims 29-53.

55. The kit according to claim 54, wherein the components (a) and (b) are in separate vials.

56. Therapeutic combination of any one of the claims 29-53 or the kit of claim 54 or 55, for use as a medicament.

57. Therapeutic combination of any one of the claims 29-53 or the kit of claim 54 or 55, for use according to any one of the claims 1-3 or 26-28.


SEQUENCE LISTING (21 sequences)

SEQ ID NO: 1 - RNA, 20 nt, synthetic construct
  misc_feature 1..20: note = DMD ASO - antisense oligonucleotide based on Homo sapiens sequence
  source 1..20: mol_type = other RNA
tcaaggaagatggcatttct

SEQ ID NO: 2 - DNA, 30 bp, synthetic construct
  misc_feature 1..30: note = DMD PMO - phosphorodiamidate morpholino oligomer based on Homo sapiens sequence
  source 1..30: mol_type = other DNA
ctccaacatcaaggaagatggcatttctag

SEQ ID NO: 3 - DNA, 25 bp, synthetic construct
  misc_feature 1..25: note = M23D PMO - phosphorodiamidate morpholino oligomer based on Mus musculus sequence
  source 1..25: mol_type = other DNA
ggccaaacctcggcttacctgaaat

SEQ ID NO: 4 - protein, 12 aa, synthetic construct
  REGION 1..12: note = tetracysteine repeat motif
HRWCCPGCCKTF

SEQ ID NO: 5 - DNA, 20 bp, synthetic construct
  misc_feature 1..20: note = primer for KM155, h53R - reverse primer based on Homo sapiens sequence
  source 1..20: mol_type = other DNA
ctccggttctgaaggtgttc

SEQ ID NO: 6 - DNA, 22 bp, synthetic construct
  misc_feature 1..22: note = primer for DM8036, h55R - reverse primer based on Homo sapiens sequence
  source 1..22: mol_type = other DNA
atcctgtaggacattggcagtt

SEQ ID NO: 7 - DNA, 20 bp, synthetic construct
  misc_feature 1..20: note = primer for KM155, h48F - forward primer based on Homo sapiens sequence
  source 1..20: mol_type = other DNA
aaaagaccttgggcagcttg

SEQ ID NO: 8 - DNA, 19 bp, synthetic construct
  misc_feature 1..19: note = primer for DM8036, h47F2 - forward primer based on Homo sapiens sequence
  source 1..19: mol_type = other DNA
tgaaactggaggacccgtg

SEQ ID NO: 9 - DNA, 21 bp, synthetic construct
  misc_feature 1..21: note = primer for DM8036, h54R - reverse primer based on Homo sapiens sequence
  source 1..21: mol_type = other DNA
ccaagaggcattgatattctc

SEQ ID NO: 10 - DNA, 18 bp, synthetic construct
  misc_feature 1..18: note = primer for KM155, h49F - forward primer based on Homo sapiens sequence
  source 1..18: mol_type = other DNA
ccagccactcagccagtg

SEQ ID NO: 11 - DNA, 19 bp, synthetic construct
  misc_feature 1..19: note = primer for KM155, h52R2 - reverse primer based on Homo sapiens sequence
  source 1..19: mol_type = other DNA
ttcttccaactggggacgc

SEQ ID NO: 12 - DNA, 19 bp, synthetic construct
  misc_feature 1..19: note = primer for DM8036, h47F - forward primer based on Homo sapiens sequence
  source 1..19: mol_type = other DNA
cccataagcccagaagagc

SEQ ID NO: 13 - DNA, 20 bp, synthetic construct
  misc_feature 1..20: note = primer for DM8036, h53R - reverse primer based on Homo sapiens sequence
  source 1..20: mol_type = other DNA
ctccggttctgaaggtgttc

SEQ ID NO: 14 - DNA, 20 bp, synthetic construct
  misc_feature 1..20: note = murine skip FW primer - forward primer based on Mus musculus sequence
  source 1..20: mol_type = other DNA
acccagtctaccaccctatc

SEQ ID NO: 15 - DNA, 22 bp, synthetic construct
  misc_feature 1..22: note = murine skip RV primer - reverse primer based on Mus musculus sequence
  source 1..22: mol_type = other DNA
ctctttatcttctgcccacctt

SEQ ID NO: 16 - DNA, 30 bp, synthetic construct
  source 1..30: mol_type = other DNA
  misc_feature 1..30: note = DMD PMO(2) - phosphorodiamidate morpholino oligomer based on Homo sapiens sequence
gtgtcaccagagtaacagtctgagtaggag

SEQ ID NO: 17 - DNA, 30 bp, synthetic construct
  source 1..30: mol_type = other DNA
  misc_feature 1..30: note = DMD PMO(3) - phosphorodiamidate morpholino oligomer based on Homo sapiens sequence
ggcagtttccttagtaaccacaggttgtgt

SEQ ID NO: 18 - DNA, 25 bp, synthetic construct
  source 1..25: mol_type = other DNA
  misc_feature 1..25: note = DMD PMO(4) - phosphorodiamidate morpholino oligomer based on Homo sapiens sequence
gttgcctccggttctgaaggtgttc

SEQ ID NO: 19 - DNA, 21 bp, synthetic construct
  source 1..21: mol_type = other DNA
  misc_feature 1..21: note = DMD PMO(5) - phosphorodiamidate morpholino oligomer based on Homo sapiens sequence
cctccggttctgaaggtgttc

SEQ ID NO: 20 - DNA, 21 bp, synthetic construct
  source 1..21: mol_type = other DNA
  misc_feature 1..21: note = primer for KM155, h50F - forward primer based on Homo sapiens sequence
aggaagttagaagatctgagc

SEQ ID NO: 21 - DNA, 21 bp, synthetic construct
  source 1..21: mol_type = other DNA
  misc_feature 1..21: note = primer for KM155, h52F - forward primer based on Homo sapiens sequence
ccccagttggaagaactcatt